(19) United States
(12) Patent Application Publication
(10) Pub. No.: US 2024/0053333 A1
(43) Pub. Date: Feb. 15, 2024

(54) CHEMICAL MODIFICATION OF ANTIBODIES AND FUNCTIONAL FRAGMENTS THEREOF

(71) Applicant: Nautilus Subsidiary, Inc., Seattle, WA (US)

(72) Inventors: Seok Ki CHOI, Palo Alto, CA (US); Julia ROBINSON, East Palo Alto, CA (US)

(21) Appl. No.: 18/364,200

(22) Filed: Aug. 2, 2023

Related U.S. Application Data
(60) Provisional application No. 63/394,907, filed on Aug. 3, 2022.

(51) Int. Cl. G01N 33/533; C07K 19/00; C07K 1/08; C07K 1/13

(52) U.S. Cl. CPC G01N 33/533; C07K 19/00; C07K 1/084; C07K 1/13

(57) ABSTRACT

Provided are antibody conjugates, including a pair of cysteines crosslinked via a two-carbon bridge covalently connecting the sulfur atoms of the cysteines. Methods for making and using antibody conjugates are also provided. The compositions and methods of the present disclosure although particularly well suited to antibodies can be extended to other analytes having sulfur moieties such as proteins obtained from biological or artificial sources.

### CROSS-REFERENCE TO RELATED APPLICATION

This application claims priority to U.S. Provisional Application No. 63/394,907, filed on Aug. 3, 2022, which is incorporated herein by reference.

### REFERENCE TO SEQUENCE LISTING

This application contains a Sequence Listing, which is being submitted electronically in XML format in accordance with the WIPO Standard ST.26 and is hereby incorporated by reference in its entirety. The XML copy, created Jul. 28, 2023, is named “50109-4015.xml” and is 1826 bytes in size.

### BACKGROUND

Naturally occurring antibodies contain four separable polypeptide chains. Two heavy chains are covalently linked by disulfide bridges and each of two light chains is covalently bridged to one of the heavy chains by a disulfide bridge. Each chain has an N-terminal variable domain (V H in the heavy chain and V_{L} in the light chain) and a constant domain at its C-terminus; the constant domain of the light chain is aligned with, and disulfide bonded to, the first constant domain of the heavy chain, and the light chain variable domain is aligned with the variable domain of the heavy chain.

The constant domains are not involved directly in binding the antibody to an epitope, but they are involved in various effector functions. The variable domains of each pair of light and heavy chains are involved directly in binding the antibody to its epitope. The domains of natural light and heavy chains have the same general structure, the so-called immunoglobulin fold, and each domain comprises four framework (FR) regions, whose sequences are somewhat conserved, connected by three hyper-variable or complementarity determining regions (CDRs). The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen binding site.

Antibodies can be engineered (genetically or biochemically) to produce a variety of functional fragments (i.e. fragments that recognize and bind epitopes). The Fᵥ fragment is a heterodimer containing only the variable domains of the heavy chain and the light chain. The Fab fragment also contains the constant domain of the light chain and the first constant domain (C_{H1}) of the heavy chain. Fab′ fragments differ from Fab fragments by the addition of a few residues at the carboxy terminus of the heavy chain C_{H1} domain including one or more cysteines from the antibody hinge region. F(ab′)₂ antibody fragments can be produced as pairs of Fab′ fragments which are between hinge cysteines.

The exquisite specificity of antibodies, and functional fragments thereof, for other biological components have led to their use in a number of contexts including, for example, therapeutic delivery of a drug payload to specific cells or tissues, detection of epitopes associated with particular diagnostic or prognostic outcomes, and identification of biological components of interest to biological research endeavors. However, antibodies and functional fragments thereof, have a level of chemical complexity that often hinders attempts to chemically modify their structures with adequate specificity and yield to facilitate practical applications. Thus, there is a need for methods of modifying antibodies (or functional fragments thereof), and modified antibodies (or functional fragments thereof) that can be used for practical applications such as therapeutics, diagnostics and research. The present disclosure satisfies this need and provides other advantages as well.

### SUMMARY

The present disclosure provides an antibody, including a pair of cysteines crosslinked via a two-carbon bridge covalently connecting the sulfur atoms of the cysteines. For example, the present disclosure provides an antibody, including a pair of cysteines crosslinked via a moiety of Formula I:

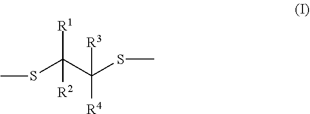

wherein S represents the sulfur moieties of the cysteines, and wherein R¹, R², R³, and R⁴ are each independently selected from any of a variety of atoms or organic moieties. In particular configurations, R¹, R², R³, and R⁴ are each independently selected from the group consisting of hydrogen, halo, alkyl, alkenyl, alkynyl, aryl, heteroalkyl, heteroalkenyl, heteroalkynyl, heteroaryl, and optionally substituted variants thereof.

The present disclosure further provides an array. The array can include a plurality of addresses and a plurality of antibodies, wherein each of the antibodies includes a pair of cysteines crosslinked via a two-carbon bridge covalently connecting the sulfur atoms of the cysteines, and wherein the two-carbon bridge further comprises a linker attached to an address of the plurality of addresses. For example, the array can include a plurality of addresses and a plurality of antibodies, wherein individual addresses of the plurality of addresses are each attached to an antibody of the plurality of antibodies, wherein each of the individual addresses is attached to a pair of modified cysteines of the antibody, the modified cysteines having a moiety of Formula I:

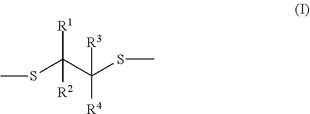

wherein S represents the sulfur moieties of the cysteines, wherein R¹, R², R³, and R⁴ are each independently selected from any of a variety of atoms or organic moieties, and wherein at least one of R¹, R², R³, and R⁴ comprises a linker connecting an antibody of the plurality of antibodies to an individual address of the plurality of addresses. Optionally, R¹, R², R³, and R⁴ are each independently selected from the group consisting of hydrogen, halo, alkyl, alkenyl, alkynyl, aryl, heteroalkyl, heteroalkenyl, heteroalkynyl, heteroaryl, and optionally substituted variants thereof.

The present disclosure provides a method of modifying an antibody by reacting an alkyne moiety of a modifying reagent with the sulfurs of a pair of cysteines, thereby forming a two-carbon bridge between the sulfurs of the pair of cysteines. For example, the method can include reacting cysteines of an antibody, with an alkyne moiety of a modifying reagent, wherein the alkyne moiety reacts with the cysteines to form a moiety of Formula I:

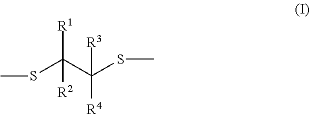

wherein S represents the sulfur moieties of the cysteines, and wherein R¹, R², R³, and R⁴ are each independently selected from any of a variety of atoms or organic moieties. Optionally, R¹, R², R³, and R⁴ are each independently selected from the group consisting of hydrogen, halo, alkyl, alkenyl, alkynyl, aryl, heteroalkyl, heteroalkenyl, heteroalkynyl, heteroaryl, and optionally substituted variants thereof.

The present disclosure provides an antibody, including a cysteine comprising a moiety of Formula II:

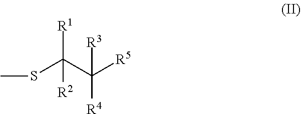

### or Formula III:

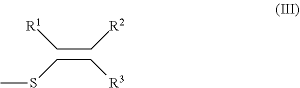

wherein S represents the sulfur moiety of the cysteine, and wherein R¹, R², R³, R⁴ and R⁵ are each independently selected from any of a variety of atoms or organic moieties. Optionally, R¹, R², R³, R⁴ and R⁵ are each independently selected from the group consisting of hydrogen, halo, alkyl, alkenyl, alkynyl, aryl, heteroalkyl, heteroalkenyl, heteroalkynyl, heteroaryl, and optionally substituted variants thereof.

Further provided herein is an array, including a plurality of addresses and a plurality of antibodies, wherein individual addresses of the plurality of addresses are each attached to an antibody of the plurality of antibodies, wherein each of the individual addresses is attached to a modified cysteine of the antibody, the modified cysteine comprising a moiety of Formula II: or Formula III:

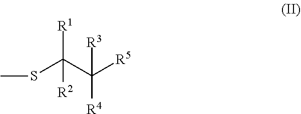

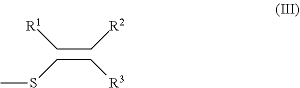

wherein S is the sulfur moiety of the cysteine, wherein R¹, R², R³, R⁴ and R⁵ are each independently selected from any of a variety of atoms or organic moieties, and wherein at least one of R¹, R², R³, R⁴ and R⁵ comprises a linker connecting an antibody of the plurality of antibodies to an individual addresses of the plurality of addresses. Optionally, R¹, R², R³, R⁴ and R⁵ are each independently selected from the group consisting of hydrogen, halo, alkyl, alkenyl, alkynyl, aryl, heteroaryl, heteroalkyl, and optionally substituted variants thereof.

Also provided herein is a method of modifying an antibody, including reacting a cysteine of an antibody, with an alkene or alkyne moiety of a modifying reagent, wherein the alkene moiety reacts with the cysteine to form a moiety of Formula II:

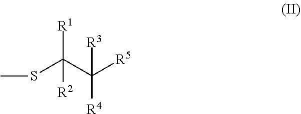

wherein the alkyne moiety reacts with the cysteine to form a moiety of Formula III:

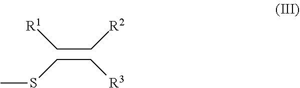

wherein S represents the sulfur moiety of the cysteine, and wherein R¹, R², R³, R⁴ and R⁵ are each independently selected from any of a variety of atoms or organic moieties. Optionally, R¹, R², R³, R⁴ and R⁵ are each independently selected from the group consisting of hydrogen, halo, alkyl, alkenyl, alkynyl, aryl, heteroalkyl, heteroalkenyl, heteroalkynyl, heteroaryl, and optionally substituted variants thereof.

### INCORPORATION BY REFERENCE

All publications, items of information available on the internet, patents, and patent applications cited in this specification are herein incorporated by reference to the same extent as if each individual publication, patent, or patent application was specifically and individually indicated to be incorporated by reference. To the extent publications, items of information available on the internet, patents, or patent applications incorporated by reference contradict the disclosure contained in the specification, the specification is intended to supersede and/or take precedence over any such contradictory material.

### BRIEF DESCRIPTION OF THE DRAWINGS

FIG. 1 shows a photo thiol-ene reaction in which light activates an alkene to form a thioether with a sulfhydryl moiety (upper) and a photo thiol-yne reaction in which light activates an alkyne to form thioethers with two sulfhydryls (lower).

FIG. 2 shows a sequence of reactions in which disulfide bridged cysteines of an IgG antibody are reduced to produce sulfhydryls and the reduced IgG is reacted with a modifying reagent having a dibenzocyclooctyne (DBCO) reactive moiety under irradiation with light (hv) to produce an IgG conjugate having four pairs of synthetically bridged cysteines (the synthetic bridge indicated by “L”).

FIG. 3 shows the sequence of reactions of FIG. 2 carried out for a Fab fragment of an antibody.

FIG. 4A shows a dibenzocyclooctyne (DBCO)-AZDye™ 647 modifying reagent.

FIG. 4B shows an SDS-PAGE gel stained with Coomassie Blue.

FIG. 4C shows a fluorescence image (Alexa 647 channel) of the SDS-PAGE gel of FIG. 4B.

FIG. 5A shows a modifying reagent having a Dibenzocyclooctyne reactive moiety linked to an oligonucleotide via a triethyleneglycol linker moiety.

FIG. 5B shows an SDS-PAGE gel stained with Coomassie Blue.

FIG. 5C shows a duplicate of the SDS-PAGE gel of FIG. 5B which was stained with a silver staining agent.

FIG. 5D shows an SDS-PAGE gel stained with Coomassie blue.

FIG. 5E shows a duplicate of the SDS-PAGE gel of FIG. 5D which was stained with SYBR gold.

FIG. 6A shows a modifying reagent having a dibenzocyclooctyne (DBCO) reactive moiety linked to methoxypolyethyleneglycol (mPEG) (MW 20,000). Modified species include primarily HL (monovalent antibody) and HLLH (divalent antibody) as marked by black arrows.

FIG. 6B shows an SDS-PAGE gel stained with Coomassie blue.

FIG. 7A shows a Propargyl-PEG4-biotin modifying reagent.

FIG. 7B and FIG. 7C show SDS-PAGE gels stained with Coomassie blue.

FIG. 8A shows an SDS-PAGE gel stained with a silver agent.

FIG. 8B shows a fluorescence image (Alexa 647 channel) of the SDS-PAGE gel of FIG. 8A.

FIG. 9A shows an SDS-PAGE gel stained with a silver agent.

FIG. 9B shows a fluorescence image (Alexa 647 channel) of the SDS-PAGE gel of FIG. 9A.

FIG. 10A shows an SDS-PAGE gel stained with a silver agent.

FIG. 10B shows a fluorescence image (Alexa 647 channel) of the SDS-PAGE gel of FIG. 10A.

FIG. 11A shows a schematic for the modification reaction of tris(2- carboxyethyl)phosphine TCEP-reduced IgG1 with polyethyleneimine(propargyl)(azide)(AF488) under irradiation at 365 nm.

FIG. 11B shows an SDS-PAGE gel stained with a silver agent.

FIG. 11C shows a fluorescence image (Alexa 488 channel) of the SDS-PAGE gel of FIG. 11B.

FIG. 12A shows a schematic for strain-promoted azide-alkyne click (SPAAC) reaction of PEI*(azide)-presenting IgG1 with DBCO-AZ647.

FIG. 12B shows an SDS-PAGE gel stained with a silver agent.

FIG. 12C shows a fluorescence image (Alexa 488 channel) of the SDS-PAGE gel of FIG. 12B.

FIG. 12D shows a fluorescence image (Alexa 647 channel) of the SDS-PAGE gel of FIG. 12B.

FIG. 12E shows a merged fluorescence image from the Alexa 488 and Alexa 647 channel of the SDS-PAGE gel of FIG. 12B.

FIG. 13A shows enzyme-linked immunosorbent assay (ELISA) results for binding of anti-tau 2N to its antigens (2N4R apo, anti-ms) and a negative control (0N4R apo).

FIG. 13B shows ELISA results for binding of anti-tau 2N to its antigens (2N4R apo, anti-ms) and a negative control (0N4R apo) after the anti-tau 2N had been treated with TCEP and iodoacetamide (IAA).

FIG. 13C shows ELISA results for binding of anti-tau 2N to its antigens (2N4R apo, anti-ms) and a negative control (0N4R apo) after the anti-tau 2N had been treated with TCEP and IAA and modified with DBCO-AZ647.

FIG. 14 shows ELISA results for binding of anti-YWL IgG₁ and modified versions to its epitope peptide (YWL).

FIG. 15 shows ELISA results for binding of anti-WNK IgG₁ and modified versions to its epitope peptide (WNK).

FIG. 16 shows a diagram of an antibody crosslinked to two other substances, objects or moieties represented by R5.

FIG. 17 shows a diagram of a synthesis of propargyl-PEG₇-azide. reagents and conditions: i) propargyl-PEG₄ acid, carbonyldiimidazole (CDI; 1.2 eq), acetonitrile, 25° C., 12 h; ii) amine-PEG₃-azide (1.1 eq), 25° C., 6 h.

FIG. 18A shows a diagram of a synthesis of propargyl-PEG₁₂-azide. reagents and conditions: i) propargyl-PEG₄ acid, N-hydroxysuccinimide (NHS, 1.2 eq), N-ethyl-N′-(3-dimethylaminopropyl)carbodiimide hydrochloride (EDC, 1.2 eq), DMSO, CHCl₃, 25° C., 12 h; ii) azide-PEG₃ amine (1.5 eq to alkyne), N,N-diisopropyl-N-ethylamine (DIPEA), 25° C., 6 h.

FIG. 18B shows the structure for propargyl-PEG₁₂-azide.

FIG. 19 shows a diagram of a reaction for photolabeling of cell lysate proteins with DBCO-AZ647 under a photo thiol-yne condition (upper) or amide conjugation of cell lysate proteins with DL650 NHS ester at lysine (lower).

FIG. 20 shows photographs of a gel after silver staining (left) or fluorescent detection at 546 nm and 650 nm) acquired after SDS-PAGE analysis of dye conjugation reactions. Lane a-b: lysate proteins (a); lysate proteins+TCEP (b); lane c-d: lysate proteins+DL650 NHS ester (50 eq, c); lysate proteins+TCEP+DL650 NHS ester (50 eq, d); lane e-h (lysate proteins+TCEP+DBCO-AZ647): no irradiation (e); 254 nm, 7 min (f); 310 nm, 7 min (g); 365 nm, 7 min (h).

FIG. 21A shows a diagram of mTz-modification of cell lysate proteins from K562 cells by photoreaction and their characterization by click conjugation with TCO-Cy5 (mTz-specific) (upper) or amide conjugation with DL650 NHS ester at lysine (lower).

FIG. 21B shows the structure for DBCO-PEG₄-mTz.

FIG. 22 shows photographs of an SDS-PAGE gel silver stained (left side) and fluorescently detected (right side) acquired after dye conjugation reactions. Legends for lanes a-h (photo mTz modification): An aliquot of lysate proteins which were photo crosslinked with DBCO-PEG₄-mTz at 365 nm was treated with DL650 NHS ester (20 eq) or TCO-Cy5 (30 eq) at 25° C. overnight prior to SDS-PAGE. Lane a-c (proteins+DL650 NHS): loaded amounts: 2× (a), 3× (b), 5× (c). Lane d-f (proteins+TCO-Cy5): loaded amounts: 2× (d), 3× (e), 5× (f). Legends for lane g-l (no mTz controls): An aliquot of the lysate proteins which were exposed to 365 nm light otherwise without DBCO-PEG₄-mTz was treated with DL650 NHS ester (20 eq) or TCO-Cy5 (30 eq) at 25° C. overnight prior to SDS-PAGE analysis. Lane g-i (proteins+DL650 NHS): loaded amounts: 2× (g), 3× (h), 5× (i). Lane j-l (proteins+TCO-Cy5): loaded amounts: 2× (j), 3× (k), 5× (l).

FIG. 23 shows a diagram of photoreaction of protein tau 0N4R (Boston Biochemical, Inc) with DBCO-PEG₄-mTz under a photo thiol-yne condition (left) and characterization reactions (right) performed for verifying mTz-modified proteins through either amide conjugation with Cy5-NHS ester at lysine (middle) or mTz-specific click reaction with TCO-Cy5 (upper) and TCO-oligonucleotide (lower).

FIG. 24 shows photographs of an SDS-PAGE gel silver stained (left side) and fluorescently detected (right side). Lane a: tau(mTz)+Cy5 NHS (5 eq); lane b: tau(mTz)+TCO-Cy5 (10 eq); lane c: tau(mTz)+Cy5 NHS (5 eq); lane d-f: tau(mTz)+Cy5 NHS (5 eq)+TCO-oligonucleotide (1 eq, d; 2 eq, e; 10 eq, f); lane g: TCO-oligonucleotide (1 eq); lane h: TCO-oligonucleotide (1 eq)+mTz-Cy5 (5 eq); lane i: mTz-Cy5 alone. Each band marked with a white arrow is attributed to a tau-oligonucleotide conjugate as depicted in FIG. 23 (lower right).

FIG. 25 shows a schematic diagram of photoreaction of cell lysate proteins with propargyl-PEG₁₂-azide under a photo thiol-yne condition and their characterization by azide-specific click conjugation with DBCO-AZ647.

FIG. 26 shows photographs of an SDS-PAGE gel silver stained (left side) and fluorescently detected (right side). Lane a: lysate proteins (unmodified)+DL650 NHS ester (20 eq), 25° C., 12 h; lane b-c: lysate proteins (photo crosslinked with alkyne-PEG12-azide)+DBCO-AZ647 (10 eq for lane b or 20 eq for lane c), 25° C., 12 h.

FIG. 27A shows a schematic diagram of biotin modification of cell lysate proteins (upper) or IgG(AF488)-spiked cell lysate proteins (lower) with DBCO-PEG₈-biotin under a photo thiol-yne condition.

FIG. 27B shows the structure for DBCO-PEG₈-biotin.

FIG. 28A shows photographs of an SDS-PAGE gel silver stained (left side) and fluorescently detected (right side), characterizing biotin-modified proteins of cell lysate proteins by complexation with streptavidin(AZ647). Lane a: lysate proteins (biotin crosslinked)+streptavidin(647) (1 eq), heated (90° C., 10 min); lane b: lysate proteins (biotin crosslinked)+streptavidin(647) (1 eq), not heated; lane c: streptavidin(647) alone (1 eq), heated. Biotin-modified proteins that are complexed with streptavidin(647) display a significant band tailing as marked with a star (lane b). However, upon sample heating (lane a), some fraction loses such tailing due to streptavidin decomplexation as evident with appearance of protein bands at relatively lower MW (double starred).

FIG. 28B shows photographs of an SDS-PAGE gel silver stained (left side) and fluorescently detected (right side), characterizing biotin-modified proteins of IgG(488)-spiked cell lysate proteins by complexation with streptavidin(647). Lane d: lysate proteins/IgG(488) (biotin crosslinked), heated (90° C., 10 min); lane e: lysate proteins/IgG(AF488) (biotin crosslinked)+streptavidin(647) (1 eq), heated; lane f: lysate proteins/IgG(AF488) (biotin crosslinked)+streptavidin(647) (1 eq), not heated; lane g: streptavidin(647) alone (1 eq), heated; lane h: streptavidin(647) alone (1 eq), not heated. Bands marked with arrows are attributed to those IgG bands shifted to higher MW species as a result of complexation with streptavidin.

FIG. 29A shows a photo crosslinking reaction that occurs between an alkyne molecule and a thiol moiety at cysteine under a photo thiol-yne condition.

FIG. 29B shows a general scheme that depicts functional modifications of proteins at cysteines by photo reaction with alkyne-spacer-x, a dual-functional molecule, under the photo thiol-yne condition.

FIG. 30A shows a reaction for covalent attachment of an mTz-modified protein at a TCO-presenting structured nucleic acid particle (SNAP) through inverse electron demand Diels-Alder (IEDDA) reaction.

FIG. 30B shows a reaction for covalent attachment of an azide-modified protein at a DBCO-presenting SNAP through strain-promoted azide-alkyne click (SPAAC) reaction.

FIG. 30C shows a reaction for non-covalent complexation of a biotin-modified protein with streptavidin (SA) presented on a SNAP.

FIG. 31A shows the structure of dibenzocyclooctyne (DBC0)-PEG₈-methyltetrazine (mTz) and that of 3-(4-acylphenyl)propiolonitrile (APN)-PEG₄-tetrazine (Tz).

FIG. 31B shows an IgG antibody conjugate, a product acquired from modification with dibenzocyclooctyne (DBCO)-PEG₈-mTz or 3-(4-acylphenyl)propiolonitrile (APN)-PEG₄-Tz through light irradiation at 365 nm. This IgG conjugate has four pairs of synthetically bridged cysteines. The synthetic bridge indicated by “L” is shown in each of the two structures provided to the right.

FIG. 31C and FIG. 31D show photographs of an SDS-PAGE gel Coomassie stained (FIG. 31C) and fluorescently detected (FIG. 31D) acquired after (m)Tz modification of an IgG antibody (FIG. 32B) and subsequent dye conjugation reaction at (m)Tz residues. In sample preparation, TCEP-treated anti-DTR IgG was modified through photo stapling with either DBCO-PEG₈-mTz or APN-PEG₄-Tz at 365 nm (FIG. 32B), and after its (m)Tz modification, the antibody was treated with TCO-Cy5 for dye conjugation at mTz residues prior to SDS-PAGE.

FIG. 32A shows a scheme for covalent conjugation of (m)Tz-modified IgG with trans-cyclooctyne (TCO)-presenting structured nucleic acid particle (SNAP), a reaction which occurs through inverse electron demand Diels-Alder (IEDDA).

FIG. 32B shows a fluorescent image of an SDS-PAGE gel (Lumitein™ stained) of the IgG-structured nucleic acid particle (SNAP) conjugates as depicted in FIG. 32A. Lane a and b: HPLC purified samples of SNAP conjugated with mTz-modified anti-HPD IgG or mTz-modified anti-HSP IgG, respectively. Lane c and d: HPLC purified samples of SNAP conjugated with Tz-modified anti-HPD IgG or Tz-modified anti-HSP IgG, respectively.

### DETAILED DESCRIPTION

The present disclosure provides protein conjugates that are modified at cysteine residues, methods for making the protein conjugates and methods for using the protein conjugates. A protein can be modified at a cysteine residue such that an exogenous moiety is attached to the cysteine. The exogenous moiety can be a label moiety, such as a luminophore, that imparts a detectable characteristic to the protein to which it is attached. Another useful moiety is an attachment moiety that is reactive with, or attached to, an object, moiety or substance of interest (e.g. a cytotoxic agent, therapeutic agent, nucleic acid, particle or solid support surface). In some cases, two different protein molecules or subunits can be crosslinked via modification of a cysteine on each of the two molecules or subunits. Antibodies (full length or fragments thereof) are particularly well suited to the modifications and methods set forth herein. For purposes of illustration, the present disclosure sets forth several features and advantages in the context of modified antibodies, their manufacture or use. It will be understood that compositions or methods set forth herein in the context of antibodies can be extended to other proteins and indeed to other molecules having reactive sulfhydryl moieties.

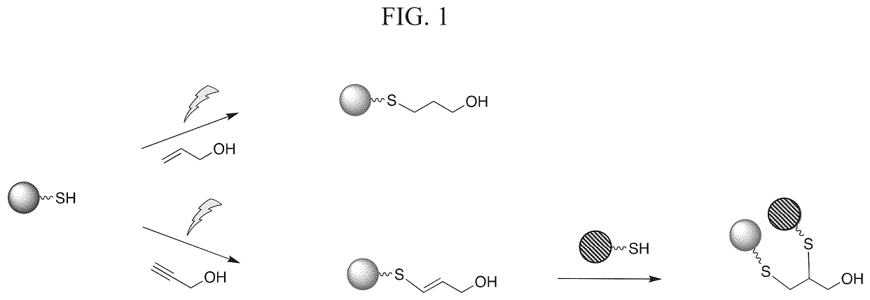

As set forth in further detail herein, the sulfhydryl of a cysteine can react with an activated alkene or alkyne to form a thioether bond. The alkene or alkyne can be activated, for example, due to its being present in a strained ring or due to light activation (e.g. a photo thiol-ene or photo thiol-yne reaction). FIG. 1 shows a photo thiol-ene reaction in which light activates an alkene to form a thioether with a sulfhydryl and a photo thiol-yne reaction in which light activates an alkyne to form thioethers with two sulfhydryls. For example, the photo thiol-yne reaction can produce a covalent bridge between sufficiently proximal cysteines, such as cysteines that are sufficiently proximate to form disulfide bridges when not in a reduced state. Alternatively, the photo thiol-yne reaction can stop at the formation of a single thioether between a single cysteine and the alkyne, for example, when the cysteine is not proximate to another sulfhydryl.

In particular configurations, antibody conjugates set forth herein include a bioorthogonal functionality that is useful for research, diagnostic or therapeutic uses. More specifically, antibody conjugates of the present disclosure can be useful as probes for protein binding assays (e.g. proteome-scale analysis), antibody-drug conjugates for disease-targeted therapeutic delivery, antibody-label conjugates for detection and imaging, and immobilized antibodies for analytical or preparative methods. An antibody conjugate can be prepared from at least two structural or functional components: (1) an antibody (e.g. full length or functional fragment thereof), and (2) a modifying reagent including an alkene or alkyne reactive moiety. Optionally, the modifying reagent can further include an attachment moiety that is configured to attach the antibody conjugate to an object, substance or moiety. In some cases, the modifying reagent can be attached to an object, substance or moiety prior to conjugation of the modifying reagent to the antibody or in a reaction that occurs during or after reaction of the antibody with the modifying reagent. Any of a variety of objects, substances or moieties can be attached to an antibody conjugate including, but not limited to, an attachment moiety, label, therapeutic agent, cytotoxic agent, nucleic acid, protein, particle, solid support, another antibody or the like.

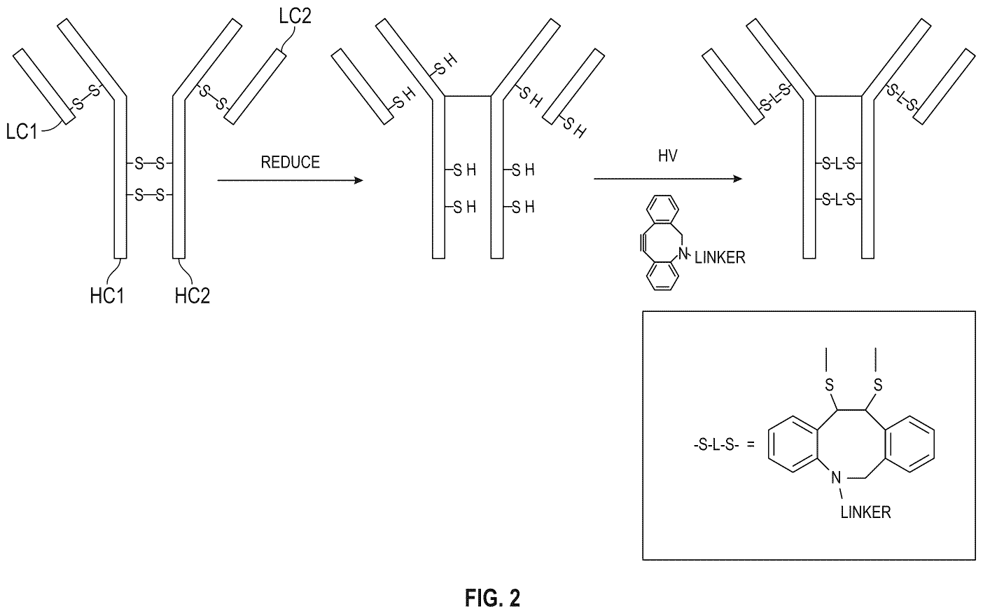

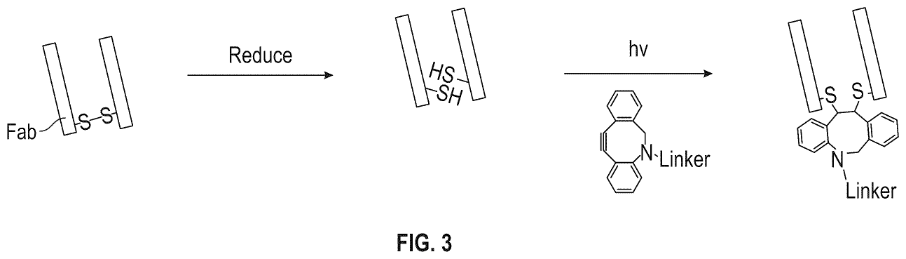

Antibodies are relatively complex molecules with a diversity of reactive residues that are available for modification to produce antibody conjugates. For example, the amino moieties of lysines are highly reactive with a number of well-known reagents. However, lysine residues are widely distributed over the antibody structure (approximately 90 per typical antibody molecule) and lysines are relatively common in the antigen binding domains of antibodies. Reactivity of lysines in proteins, such as antibodies, can be sensitive to local environment such as the polarity or local pH resulting from the number and type of amino acid residues near the lysines in the proteins. As such, conjugation of lysines can produce a wide variety of reaction products, thereby thwarting attempts to reproducibly manufacture high quality conjugates, and in many cases, conjugation can inactivate antibodies by making unwanted modifications to the antigen binding domain. The use of cysteines for mediating conjugation to an antibody provides several advantages over other protein conjugation methods. These advantages can be understood in the context of the diagrammatic example of FIG. 2. First, antibodies generally have only a few cysteines, most of which are located at regions that are conserved across antibody structures. As exemplified by the IgG antibody in FIG. 2, cysteines are located in the flexible hinge region (Fc) such that disulfide bridges are located at the interface between the two heavy chains heavy (HC1 and HC2). The IgG also has disulfide bridges between a cysteine in each heavy chain and a cysteine in each light chain (LC1 or LC2, respectively). The disulfides can be reduced to produce sulfhydryls as shown in the first step of FIG. 2. Then, the reduced antibody can be reacted with a h having a regular alkyne or an activated alkyne such as dibenzocyclooctyne (DBCO) reactive moiety under irradiation with light (hv). The product of the photo thiol-yne reaction includes an IgG conjugate having four pairs of synthetically bridged cysteines (the synthetic bridge indicated by “L”). Fewer synthetic bridges can be formed by altering conditions of the reaction, for example, using a stoichiometric limiting amount of modifying reagent relative to antibody. Accordingly, cysteine conjugation can provide a more targeted conjugation of an antibody compared to other conjugation chemistries in terms of the uniformity and reproducibility of conjugation products. Moreover, photo thiol-yne conjugation can provide the added advantage of creating a synthetic bridge between cysteines that are typically connected by a disulfide bridge. This is particularly advantageous for maintaining association between antibody subunits under reductive conditions or environments where an undesirable dissociation can occur. A similar method can be performed to form a synthetic bridge between cysteine pairs in any of a variety of antibody fragments, as exemplified for a Fab in FIG. 3.

Further benefits of the conjugation methods of the present disclosure include convenience of performing the methods, high conjugation efficiency and availability of a diverse range of alkene and alkyne reagents. Additionally, alkyne or alkene reactive moieties are significantly more stable than other handles that permit direct conjugation with a specific amino-acid side chain (e.g. maleimides, activated esters). This allows for a much more efficient ‘direct’ conjugation between an alkyne or alkene presenting modifying reagent that does not require an added step to functionalize the antibody itself. Typical directly protein-reactive attachment strategies (e.g. maleimides, activated esters) are subject to side-reactions in aqueous buffers, such as hydrolysis, which result in loss of the valuable nanostructures as non-conjugated entities. The chemistry set forth herein can provide a much higher yielding process for modifying antibodies and other molecules having sulfhydryl moieties. The direct conjugation furthermore reduces the likelihood that unnecessary modifications to the antibody or protein will adversely affect downstream uses such as diagnostic or therapeutic applications.

Terms used herein will be understood to take on their ordinary meaning in the relevant art unless specified otherwise. Several terms used herein and their meanings are set forth below.

As used herein, the term “address” refers to a location in an array where a particular analyte (e.g. protein, antibody or nucleic acid) is present. An address can contain a single analyte, or it can contain a population of several analytes of the same species (i.e. an ensemble of the analytes). Alternatively, an address can include a population of different analytes. Addresses are typically discrete. The discrete addresses can be contiguous, or they can be separated by interstitial spaces. An array useful herein can have, for example, addresses that are separated by less than 100 microns, 10 microns, 1 micron, 100 nm, 10 nm or less. Alternatively or additionally, an array can have addresses that are separated by at least 10 nm, 100 nm, 1 micron, 10 microns, or 100 microns. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses.

As used herein, the term “affinity reagent” refers to a molecule or other substance that is capable of specifically or reproducibly binding to an analyte (e.g. protein). An affinity reagent can be larger than, smaller than or the same size as the analyte. An affinity reagent may form a reversible or irreversible bond with an analyte. An affinity reagent may bind with an analyte in a covalent or non-covalent manner. Affinity reagents may include reactive affinity reagents, catalytic affinity reagents (e.g., kinases, proteases, etc.) or non-reactive affinity reagents (e.g., antibodies or functional fragments thereof). An affinity reagent can be non-reactive and non-catalytic, thereby not permanently altering the chemical structure of an analyte to which it binds. A particularly useful affinity reagent is an antibody, such as a full-length antibody or functional fragment thereof. A functional fragment of an antibody can include any fragment that is capable of binding to an epitope with a detectable affinity, such as a Fab, Fab′, F(ab′)₂, single-chain variable (scFv), di-scFv, tri-scFv, or microantibody. Other useful affinity reagents include, for example, affibodies, affilins, affimers, affitins, alphabodies, anticalins, avimers, DARPins, monobodies, nanoCLAMPs, nucleic acid aptamers, protein aptamers, lectins or functional fragments thereof.

As used herein, the term “alkyl” refers to a straight or branched hydrocarbon chain that comprises a fully saturated (no double or triple bonds) hydrocarbon group. Optionally, an alkyl moiety may have at least 1, 2, 3, 4, 5, 6, 7, 8, 9, 10, 15 or 20 carbon atoms. Alternatively or additionally, an alkyl moiety may have at most 20, 15, 10, 9, 8, 7, 6, 5, 4, 3, 2 or 1 carbon atoms. The alkyl group of the compounds may be designated as “C1-C4 alkyl” which indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, and hexyls. An alkyl moiety may be substituted or unsubstituted. An alkyl moiety can be linear or cyclic. For example, the term “cycloalkyl” refers to a completely saturated (no double or triple bonds) mono- or multi-cyclic hydrocarbon ring system. When composed of two or more rings, the rings may be joined together in a fused fashion. Cycloalkyl moieties can contain at least 3, 4, 5, 6, 7, 8, 9, 10 or more atoms in the ring(s). Alternatively or additionally, cycloalkyl moieties can contain at most 10, 9, 8, 7, 6, 5, 4, or 3 atoms in the ring(s).

As used herein, the term “alkenyl” or “alkene” refers to a straight or branched hydrocarbon chain that includes one or more carbon-carbon double bonds. An alkenyl moiety may be unsubstituted or substituted. An alkenyl moiety can be linear or cyclic. For example, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). When composed of two or more rings, the rings may be connected together in a fused fashion. A cycloalkenyl moiety may be unsubstituted or substituted.

As used herein, the term “alkynyl” or “alkyne” refers to a straight or branched hydrocarbon chain that includes one or more triple bonds. An alkynyl moiety may be unsubstituted or substituted. An alkynyl moiety can be linear or cyclic. For example, the term “cycloalkynyl” refers to a mono- or multi-cyclic hydrocarbon ring system that contains one or more triple bonds in at least one ring. If there is more than one triple bond, the triple bonds cannot form a fully delocalized pi-electron system throughout all the rings. When composed of two or more rings, the rings may be joined together in a fused fashion.

As used herein, the term “alkoxy” refers to the formula —OR, wherein R is an alkyl, an alkenyl, an alkynyl, a cycloalkyl, a cycloalkenyl or a cycloalkynyl is defined herein. A non-limiting list of alkoxys is methoxy, ethoxy, n-propoxy, 1-methylethoxy (isopropoxy), n-butoxy, iso-butoxy, sec-butoxy, and tert-butoxy. An alkoxy may be substituted or unsubstituted.

As used herein, the term “alkylamino” refers to an alkyl moiety in which one or more of the hydrogen atoms are replaced by an amino group. Exemplary alkylamino groups include but are not limited to aminomethyl, 2-aminoethyl, 3-aminoethyl. An alkylamino may be substituted or unsubstituted.

As used herein, the term “alkylamido” refers to an alkyl group in which one or more of the hydrogen atoms are replaced by a C-amido group or an N-amido group. An alkylamido may be substituted or unsubstituted.

As used herein, the term “alkylthio” refers to RS—, in which R is an alkyl. Alkylthio can be substituted or unsubstituted.

As used herein, the term “amine” refers to a basic nitrogen atom with a lone pair of electrons. A primary amine has structure RNH₂, a secondary amine has structure R₂NH, and a tertiary amine has structure R₃N. R can be, for example, independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl.

As used herein, the term “aldehyde” refers to a —R—C(O)H group, wherein R can be absent or independently selected from alkylene, alkenylene, alkynylene, cycloalkylene, cycloalkenylene, cycloalkynylene, arylene, heteroarylene, heteroalicyclylene, aralkylene, or (heteroalicyclyl)alkylene.

As used herein, the term “array” refers to a population of analytes (e.g. proteins or antibodies) that are associated with unique identifiers such that the analytes can be distinguished from each other. A unique identifier can be, for example, a solid support (e.g. particle or bead), address on a solid support, tag, label (e.g. luminophore), or barcode (e.g. nucleic acid barcode) that is associated with an analyte and that is distinct from other identifiers in the array. Analytes can be associated with unique identifiers by attachment, for example, via covalent bonds or non-covalent bonds (e.g. ionic bond, hydrogen bond, van der Waals forces, electrostatics etc.). An array can include different analytes that are each attached to different unique identifiers. An array can include different unique identifiers that are attached to the same or similar analytes. An array can include separate solid supports or separate addresses that each bear a different analyte, wherein the different analytes can be identified according to the locations of the solid supports or addresses.

As used herein, the term “aryl” refers to a carbocyclic (all carbon) monocyclic or multicyclic aromatic ring system (including, e.g., fused, bridged, or spiro ring systems where two carbocyclic rings share a chemical bond, e.g., one or more aryl rings with one or more aryl or non-aryl rings) that has a fully delocalized pi-electron system throughout at least one of the rings. The number of carbon atoms in an aryl group can vary. For example, in some embodiments, the number of atoms in the ring structure can be in the range set forth above for cycloalkyl moieties. Examples of aryl moieties include, but are not limited to, benzene, naphthalene, and azulene. An aryl group may be substituted or unsubstituted.

As used herein, the term “attached” refers to the state of two things being joined, fastened, adhered, connected or bound to each other. Attachment can be covalent or non-covalent. For example, a particle can be attached to a protein by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions, adhesion, adsorption, and hydrophobic interactions.

As used herein, the term “click reaction” refers to single-step, thermodynamically-favorable conjugation reaction utilizing biocompatible reagents. A click reaction may be configured to not utilize toxic or biologically incompatible reagents (e.g., acids, bases, heavy metals) or to not generate toxic or biologically incompatible byproducts. A click reaction may utilize an aqueous solvent or buffer (e.g., phosphate buffer solution, Tris buffer, saline buffer, MOPS, etc.). A click reaction may be thermodynamically favorable if it has a negative Gibbs free energy of reaction, for example a Gibbs free energy of reaction of less than about −5 kiloJoules/mole (kJ/mol), −10 kJ/mol, −25 kJ/mol, −100 kJ/mol, −250 kJ/mol, −500 kJ/mol, or less. Exemplary click reactions may include metal-catalyzed azide-alkyne cycloaddition, strain-promoted azide-alkyne cycloaddition, strain-promoted azide-nitrone cycloaddition, strained alkene reactions, thiol-ene reaction, Diels-Alder reaction, inverse electron demand Diels-Alder reaction (IEDDA), [3+2] cycloaddition, [4+1] cycloaddition, nucleophilic substitution, dihydroxylation, thiol-yne reaction, photoclick, nitrone dipole cycloaddition, norbornene cycloaddition, oxanobornadiene cycloaddition, tetrazine ligation, and tetrazole photoclick reactions. Exemplary reactive moieties utilized to perform click reactions may include alkenes, alkynes, azides, epoxides, amines, thiols, nitrones, isonitriles, isocyanides, aziridines, activated esters, and tetrazines. Other well-known click conjugation reactions may be used having complementary bioorthogonal reaction species, for example, where a first click component comprises a hydrazine moiety and a second click component comprises an aldehyde or ketone group, and where the product of such a reaction comprises a hydrazone functional group or equivalent. Exemplary bioorthogonal and click reactions are set forth in US Pat. App. Pub. No. 2021/0101930 A1, which is incorporated herein by reference.

The term “comprising” is intended herein to be open-ended, including not only the recited elements, but further encompassing any additional elements.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “epitope” refers to an affinity target within a protein, polypeptide or other analyte. Epitopes may include amino acid sequences that are sequentially adjacent in the primary structure of a protein. Epitopes may include amino acids that are structurally adjacent in the secondary, tertiary or quaternary structure of a protein despite being non-adjacent in the primary sequence of the protein. An epitope can be, or can include, a moiety of protein that arises due to a post-translational modification, such as a phosphate, phosphotyrosine, phosphoserine, phosphothreonine, or phosphohistidine. An epitope can optionally be recognized by or bound to an antibody. However, an epitope need not necessarily be recognized by any antibody, for example, instead being recognized by an aptamer, mini-protein or other affinity reagent. An epitope can optionally bind an antibody to elicit an immune response. However, an epitope need not necessarily participate in, nor be capable of, eliciting an immune response.

As used herein, the term “exogenous,” when used in reference to a moiety of a molecule, means the moiety is not present in a natural analog of the molecule. For example, an exogenous label of an amino acid is a label that is not present on a naturally occurring amino acid. Similarly, an exogenous label that is present on an antibody is not found on the antibody in its native milieu.

As used herein, the term “fluid-phase,” when used in reference to a molecule, means the molecule is in a state wherein it is mobile in a fluid, for example, being capable of diffusing through the fluid.

As used herein, the terms “group” and “moiety” are intended to be synonymous when used in reference to the structure of a molecule. The terms refer to a component or part of the molecule. The terms do not necessarily denote the relative size of the component or part compared to the rest of the molecule, unless indicated otherwise. A group or moiety can include one or more atoms.

As used herein, the term “halogen atom”, “halogen” or “halo” means any one of the radio-stable atoms of column 7 of the Periodic Table of the Elements, such as, fluorine, chlorine, bromine, and iodine.

As used herein, the term “heteroaryl” refers to a monocyclic or multicyclic aromatic ring system (a ring system having a least one ring with a fully delocalized pi-electron system) that contain(s) one or more heteroatoms, that is, an element other than carbon, including but not limited to, nitrogen, oxygen, and sulfur, and at least one aromatic ring. The number of atoms in the ring(s) of a heteroaryl moiety can vary. For example, in some embodiments, a heteroaryl moiety can contain at least 4, 6, 8, 10, 12, 14, 16, 18, 20 or more atoms in the ring(s). Alternatively or additionally, a heteroaryl moiety can contain at most 20, 18, 16, 14, 12, 10, 8, 6 or 4 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline, and triazine. A heteroaryl group may be substituted or unsubstituted.

As used herein, the term “heterocyclyl” refers to ring systems including at least one heteroatom (e.g., O, N, S). Such systems can be unsaturated, can include some unsaturation, or can contain some aromatic portion, or be all aromatic. A heterocyclyl group may be unsubstituted or substituted.

As used herein, the term “hydroxy” as used herein refers to a —OH group.

As used herein, the term “immobilized,” when used in reference to a molecule that is in contact with a fluid phase, refers to the molecule being prevented from diffusing in the fluid phase. For example, immobilization can occur due to the molecule being confined at, or attached to, a solid phase. Immobilization can be temporary (e.g. for the duration of one or more steps of a method set forth herein) or permanent. Immobilization can be reversible or irreversible under conditions utilized for a method, system or composition set forth herein.

As used herein, the term “label” refers to a molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a luminophore (e.g. fluorophore), chromophore, nanoparticle (e.g., gold, silver, carbon nanotubes, quantum dots, upconversion nanocrystals), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., a nucleic acid barcode) or in a time-resolved manner (e.g., time-resolved fluorescence). A label may produce a signal with a characteristic frequency, intensity, polarity, duration, wavelength, sequence, or fingerprint.

As used herein, the term “linker” refers to a moiety that connects objects, substances or moieties to each other. Exemplary objects, substances or moieties include, but are not limited to, an antibody or other affinity reagent, a nucleic acid such as a structured nucleic acid particle (SNAP), protein, molecule, solid support, address of an array, particle or bead. The term can also refer to an atom, moiety or molecule that is configured to react with objects, substances or moieties to connect them. The connection of a linker to an object, substance or moiety can be a covalent bond or non-covalent bond. A linker may be configured to provide a chemical or mechanical property to the moiety connecting the objects, substances or moieties, such as hydrophobicity, hydrophilicity, electrical charge, polarity, rigidity, or flexibility. A linker may include two or more functional groups that facilitate coupling of the linker to the objects, substances or moieties. A linker may include a polyfunctional linker such as a homobifunctional linker, heterobifunctional linker, homopolyfunctional linker, or heteropolyfunctional linker. Exemplary moieties that can be included in a linker include, but are not limited to, polyethylene glycol (PEG), polyethylene oxide (PEO), polyethyleneimine (PEI), amino acid, protein, nucleotide, nucleic acid, nucleic acid origami, dendrimer, dendron, protein nucleic acid (PNA), polysaccharide, carbon, nitrogen, oxygen, ether, sulfur, or disulfide.

As used herein, the term “nucleic acid origami” refers to a nucleic acid construct having an engineered tertiary or quaternary structure. A nucleic acid origami may include DNA, RNA, PNA, modified or non-natural nucleic acids, or combinations thereof. A nucleic acid origami may include a plurality of oligonucleotides that hybridize via sequence complementarity to produce the engineered structuring of the origami. A nucleic acid origami may include sections of single-stranded or double-stranded nucleic acid, or combinations thereof. Exemplary nucleic acid origami structures may include nanotubes, nanowires, cages, tiles, nanospheres, blocks, and combinations thereof. A nucleic acid origami can optionally include a relatively long scaffold nucleic acid to which multiple smaller nucleic acids hybridize, thereby creating folds and bends in the scaffold that produce an engineered structure. The scaffold nucleic acid can be circular or linear. The scaffold nucleic acid can be single stranded but for hybridization to the smaller nucleic acids. A smaller nucleic acid (sometimes referred to as a “staple”) can hybridize to two regions of the scaffold, wherein the two regions of the scaffold are separated by an intervening region that does not hybridize to the smaller nucleic acid.

As used herein, the term “orthogonal,” when used in reference to two or more reactants, refers to the two or more reactants having mutually non-competing reactivity. Similarly, two or more reactions are referred to as being orthogonal when (1) the reagents or products of a first reaction are inert to reagents and products of a second reaction, and (2) the reagents or products of the second reaction are inert to reagents and products of the first reaction. An orthogonal reaction may be further characterized as being inert to components of a biological system other than those targeted by the reactants. An orthogonal reaction may utilize an enzymatic reaction, such as attachment between a first molecule and a second molecule by an enzyme such as a sortase, a ligase, or a subtiligase. An orthogonal reaction may utilize an irreversible peptide capture system, such as SpyCatcher/SpyTag, SnoopCatcher/SnoopTag, or SdyCatcher/SdyTag.

As used herein, the term “protein” refers to a molecule comprising two or more amino acids joined by a peptide bond. A protein may also be referred to as a polypeptide, oligopeptide or peptide. A protein can be a naturally-occurring molecule, or synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications. In some circumstances, different proteins may be distinguished from each other based on different genes from which they are expressed in an organism, different primary sequence length or different primary sequence composition. Proteins expressed from the same gene may nonetheless be different proteoforms, for example, being distinguished based on non-identical length, non-identical amino acid sequence or non-identical post-translational modifications. Different proteins can be distinguished based on one or both of gene of origin and proteoform state.

As used herein, any “R” groups (or moieties) such as, without limitation, R¹, R², R³, R⁴, R⁵, etc. represent substituents that can be attached to the indicated atom. An R group may be substituted or unsubstituted. If two “R” groups are described as being “taken together” the R groups and the atoms they are attached to can form a cycloalkyl, aryl, heteroaryl, or heterocycle. For example, without limitation, if R², R³, and an atom to which they are attached, are indicated to be “taken together” or “joined together” it means that they are covalently bonded to one another to form a ring.

As used herein, the term “substituted,” when used in reference to a particular moiety, refers to one or more hydrogen atoms of the moiety being another atom or moiety instead. The other atom or moiety can be, for example, hydroxyl, thiol, alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, alkoxy, aryloxy, acyl, mercapto, alkylthio, arylthio, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, amino, mono-substituted amino group, or di-substituted amino group.

As used herein, the term “single,” when used in reference to an object such as an analyte, means that the object is individually manipulated or distinguished from other objects. A single analyte can be a single molecule (e.g. single protein), a single complex of two or more molecules (e.g. a multimeric protein having two or more separable subunits, a single protein attached to a structured nucleic acid particle or a single protein attached to an affinity reagent), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

As used herein, the term “single-analyte resolution” refers to the detection of, or ability to detect, an analyte on an individual basis, for example, as distinguished from its nearest neighbor in an array.

As used herein, the term “solid support” refers to a substrate that is insoluble in aqueous liquid. Optionally, the substrate can be rigid. The substrate can be non-porous or porous. The substrate can optionally be capable of taking up a liquid (e.g. due to porosity) but will typically, but not necessarily, be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In particular configurations, a flow cell contains the solid support such that fluids introduced to the flow cell can interact with a surface of the solid support to which one or more components of a binding event (or other reaction) is attached.

As used herein, the term “structured nucleic acid particle” or “SNAP” refers to a single- or multi-chain polynucleotide molecule having a compacted three-dimensional structure. The compacted three-dimensional structure can optionally be characterized in terms of hydrodynamic radius or Stoke's radius of the SNAP relative to a random coil or other non-structured state for a nucleic acid having the same mass or sequence length as the SNAP. The compacted three-dimensional structure can optionally be characterized with regard to tertiary structure. For example, a SNAP can be configured to have an increased number of internal binding interactions between regions of a polynucleotide strand, less distance between the regions, increased number of bends in the strand, and/or more acute bends in the strand, as compared to a nucleic acid molecule of similar length in a random coil or other non-structured state. Alternatively or additionally, the compacted three-dimensional structure can optionally be characterized with regard to tertiary or quaternary structure. For example, a SNAP can be configured to have an increased number of interactions between polynucleotide strands or less distance between the strands, as compared to a nucleic acid molecule of similar length in a random coil or other non-structured state. In some configurations, the secondary structure of a SNAP can be configured to be more dense than a nucleic acid molecule of similar length in a random coil or other non-structured state. A SNAP may contain DNA, RNA, PNA, modified or non-natural nucleic acids, or combinations thereof. A SNAP may include a plurality of oligonucleotides that hybridize to form the SNAP structure. The plurality of oligonucleotides in a SNAP may include oligonucleotides that are attached to other molecules (e.g., probes, analytes such as proteins, reactive moieties, or detectable labels) or are configured to be attached to other molecules (e.g., by functional groups). A SNAP may include engineered or rationally designed structures. Exemplary SNAPs include nucleic acid origami and nucleic acid nanoballs.

The embodiments set forth below and recited in the claims can be understood in view of the above definitions.

The present disclosure provides an antibody, including a pair of cysteines crosslinked via a two-carbon bridge covalently connecting the sulfur atoms of the cysteines. For example, the present disclosure provides an antibody, including a pair of cysteines crosslinked via a moiety of Formula I:

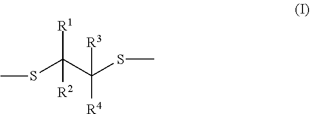

wherein S represents the sulfur moieties of the cysteines, and wherein R¹, R², R³, and R⁴ are each independently selected from any of a variety of atoms or organic moieties. In particular configurations, R¹, R², R³, and R⁴ are each independently selected from the group consisting of hydrogen, halo, alkyl, alkenyl, alkynyl, aryl, heteroalkyl, heteroalkenyl, heteroalkynyl, heteroaryl, and optionally substituted variants thereof.

An antibody can be any antigen-binding molecule or molecular complex having at least one complementarity determining region (CDR) that binds to or interacts with a particular antigen with high affinity. An antibody can include four polypeptide chains: two heavy chains (HC1 and HC2) and two light chains (LC1 and LC2). HC1 and HC2 can be covalently connected by one, two or more disulfide bonds. HC1 can be covalently connected to LC1 by at least one disulfide bond. HC2 can be covalently connected to LC2 by at least one disulfide bond. Each heavy chain can include a heavy chain variable region (V_{H}) and a heavy chain constant region (C_{H}). The heavy chain constant region can include three domains, C_{H1}, C_{H2} and C_{H3}. Each light chain can include a light chain variable region (V_{L}) and a light chain constant region (C_{L}). The V_{H} and V_{L} regions can further include regions of hypervariability, termed complementarity determining regions (CDRs), interspersed with regions that are more conserved, termed framework regions (FR). Each V_{H} and V_{L} can include three CDRs and four FRs, arranged from amino-terminus to carboxy-terminus in the following order: FR1, CDR1, FR2, CDR2, FR3, CDR3, FR4.

An antibody can include all elements of a full-length antibody, such as those enumerated above. However, an antibody need not be full length and functional fragments can be particularly useful for many uses. The term “antibody” is used herein to encompass full length antibodies and functional fragments thereof. A functional fragment can be naturally occurring, enzymatically obtainable, synthetic, or genetically engineered. An antibody can be obtained using any suitable technique such as proteolytic digestion or recombinant genetic engineering techniques involving the manipulation and expression of DNA encoding one or more antibody domains. Such DNA is readily available, for example, from commercial sources, DNA libraries (e.g., phage-antibody libraries), or can be synthesized. The DNA may be sequenced and manipulated chemically or by using molecular biology techniques, for example, to arrange one or more variable and/or constant domains into a suitable configuration, or to introduce codons, introduce cysteine residues, remove cysteine residues, modify, add or delete other amino acids, etc.

A functional fragment of an antibody can include any fragment that is capable of binding to an epitope with a detectable affinity, such as a Fab, Fab′, F(ab′)₂, Fd, Fv, dAb, single-chain variable (scFv), di-scFv, tri-scFv, microantibody, or minimal recognition unit consisting of the amino acid residues that mimic the hypervariable region of an antibody (e.g., an isolated complementarity determining region (CDR) such as a CDR3 peptide). Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains can also be useful.

A functional fragment of an antibody will typically include at least one variable domain. The variable domain may be of any size or amino acid composition and will generally include at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, a functional fragment of an antibody may contain a monomeric V_{H} or V_{L} domain.

In particular configurations, a functional fragment of an antibody contains at least one variable domain covalently connected to at least one constant domain. Non-limiting, exemplary configurations of variable and constant domains that may be found within an antigen-binding fragment of an antibody of the present disclosure include: (i) V_{H}-C_{H1}; (ii) V_{H}-C_{H2}; (H) V_{H}-C_{H3}; (iv) V_{H}-C_{H1}-C_{H2}; (v) V_{H}-C_{H1}-C_{H2}-C_{H3}; (vi) V_{H}-C_{H2}-C_{H3}; (vii) V_{H}-C_{L}; (viii) V_{L}-C_{H1}; (ix) V_{L}-C_{H2}; (x) V_{L}-C_{H3}; (xi) V_{L}-C_{H2}; (xii) V_{L}-C_{H1}-C_{H2}-C_{H3}; (xiii) V_{L}-C_{H2}-C_{H3}; and (xiv) V_{L}-C_{L}. In any configuration of variable and constant domains, including any of the exemplary configurations listed above, the variable and constant domains may be either directly connected to one another or may be connected by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody may include a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full-length antibody molecules, functional fragments may be monospecific or multispecific (e.g., bispecific). A multispecific functional fragment of an antibody will typically include at least two different variable domains, wherein each variable domain is capable of binding to a separate antigen or to a different epitope on the same antigen. Any multispecific antibody format may be adapted for use in the context of an antigen-binding fragment of an antibody of the present disclosure using routine techniques available in the art.

An antibody of the present disclosure can include a pair of cysteines that are positioned to participate in a disulfide bridge or synthetic bridge (e.g. a two-carbon bridge). One or both cysteines of the pair may be in a reduced state due to the chemical environment (e.g. redox state, reduction state, or presence of reducing agents such as thioredoxin, tris(2-carboxyethyl)phosphine (TCEP), 1,4-dithio-2-butylamine (DTBA) or dithiothreitol (DTT)) and/or due to the cysteines being separated due to denaturation or dissociation of the antibody. The cysteines of the pair may, nevertheless, be capable of forming a disulfide bridge or synthetic bridge when in the appropriate chemical environment and when the cysteines are in proximity to each other. Optionally, a pair of cysteines that participates in a disulfide bridge or synthetic bridge can include a sulfur moiety on a heavy chain of an antibody and a sulfur moiety on a light chain of an antibody. Alternatively or additionally, a pair of cysteines that participates in a disulfide bridge or synthetic bridge can include a sulfur moiety on a first heavy chain of an antibody and a sulfur moiety on a first light chain of an antibody. Pairs of cysteines that are found in other proteins, whether forming disulfide bridges within a single polypeptide chain or between subunits formed by separable chains, can be in a redox state exemplified herein for antibodies or can be converted from one redox state to another as exemplified herein for antibodies.

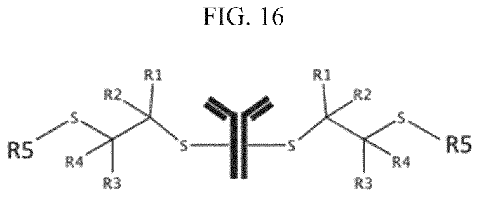

Several aspects of the present methods and compositions are exemplified in the context of a crosslink between cysteines of a pair located within a given antibody subunit or between cysteines of a pair located on two different subunits of an antibody. However, the compositions or methods of the present disclosure can utilize a crosslink between a sulfur on a first antibody and a sulfur on another substance or object. For example, FIG. 16 shows a diagram of an antibody crosslinked to two other substances, objects or moieties (the other substances, moieties or objects being indicated by R5). One or both R5 moieties can be independently selected from the substances, objects or moieties set forth herein. For example, the R5 moieties can be antibodies. As such, crosslinks can be formed between multiple antibodies to form a polymer of antibodies. Two or more antibodies that are crosslinked can have the same composition as each other or, alternatively, their compositions can differ. Two or more antibodies that are crosslinked can recognize or bind to the same antigens/epitopes as each other or, alternatively the two or more antibodies can recognize or bind to different antigens/epitopes. The R1, R2, R3 and R4 moieties can be selected from those set forth herein in the context of Formula I.

In some configurations an antibody will include only a single pair of cysteines that participates in a disulfide bridge or synthetic bridge. However, multiple pairs of cysteines can participate in a disulfide bridge or synthetic bridge in an antibody set forth herein. For example, an antibody can include any combination of pairs of cysteines that participates in a disulfide bridge or synthetic bridge within a single HC, between a first HC and second HC, within a single LC, or between an HC and an LC. Alternatively or additionally, the antibody can include two or more pairs of cysteines that participate in disulfide bridges or synthetic bridges within a single HC. Moreover, the antibody can include two or more pairs of cysteines that participate in disulfide bridges or synthetic bridges within a single LC. An antibody can include at least 1, 2, 3, 4 or more pairs of cysteines that participate in a disulfide bridge or synthetic bridge. Alternatively or additionally, an antibody can include at most 4, 3, 2, or 1 pairs of cysteines that participate in a disulfide bridge or synthetic bridge. The cysteines that participate in a disulfide bridge or synthetic bridge can be native cysteines (e.g. cysteines that are conserved in the antibody structure). Alternatively, one or both of the cysteines in a pair of cysteines that participate in a disulfide bridge or synthetic bridge can be exogenous, for example, having been introduced via genetic engineering or synthetic techniques.

Formula I can form a linear, non-cyclic structure. For example, the alkyl, alkenyl, alkynyl, aryl, heteroalkyl, heteroalkenyl, heteroalkynyl, heteroaryl or substituted variant of Formula I can form a linear structure. Alternatively, Formula I can form a cyclic structure. For example, two or more of R¹, R², R³, and R⁴ of Formula I can occupy a ring structure.

A pair of cysteines can be bridged via a dibenzocyclooctane moiety. For example, FIG. 2 and FIG. 3 show pairs of cysteines linked via bonding of their sulfurs to the eight-membered ring of a dibenzocyclooctane moiety, the product of reaction with a linker having a dibenzocyclooctyne (DBCO) moiety.

A pair of cysteines can be bridged via a linear ethyl moiety. For example, a linear ethyl linkage can be produced by reaction of a pair of cysteines with a propargyl moiety, whereby the sulfurs of the cysteines are bonded to the ethyl. The ethyl can be substituted with an amido moiety, for example, as a product of reaction of a pair of cysteines with the propargyl of a modifying reagent having an amido propargyl moiety. Variants of Formula I exemplified herein in the context of antibodies can also be present or formed in proteins or other analytes subjected to methods set forth herein.

An antibody conjugate of the present disclosure can include an attachment moiety connected to a pair of synthetically bridged cysteines. For example, one or more of R¹, R², R³, and R⁴ of Formula I can be connected to an attachment moiety. The attachment moiety can be chemically reactive moiety. For example, the chemically reactive moiety can be configured to covalently react with a compatible moiety of an object, substance or moiety that is to be attached to the antibody conjugate. Examples of chemically reactive attachment moieties are set forth below. In some configurations, an attachment moiety is a binding moiety. A binding moiety can be configured to form a non-covalent bond with a compatible binding partner of an object, substance or moiety that is to be attached to the antibody conjugate. Examples of binding moieties include antibodies, other affinity reagents set forth herein and those set forth below.

An attachment moiety can include a component of a SpyTag/SpyCatcher system (See, Zakeri et al. Proceedings Nat'l Acad. Sciences USA. 109 (12):E690-7 (2012)). In this system, a 13 amino acid tag polypeptide (Spy Tag) forms a first coupling handle, with a 12.3 kDa protein (Spy-Catcher) forming the other coupling handle. Optionally, the Spy Catcher can be attached to an antibody conjugate. The Spy Catcher can function as an attachment moiety by irreversibly bonding to a Spy Tag through an isopeptide bond. As will be appreciated, either the Spy Tag or the Spy Catcher can be connected to an antibody conjugate and used as an attachment moiety.

In some configurations, the attachment moiety can be reactive in a click reaction. Attachment can be accomplished in part by chemical reaction of a click moiety with a chemically active site on an object, substance or moiety. The chemical conjugation may proceed via an amide formation reaction, reductive amination reaction, N-terminal modification, thiol Michael addition reaction, disulfide formation reaction, copper(I)-catalyzed alkyne-azide cycloaddition (CuAAC) reaction, strain-promoted alkyne-azide cycloaddtion reaction (SPAAC), Strain-promoted alkyne-nitrone cycloaddition (SPANC), inverse electron-demand Diels-Alder (IEDDA) reaction, oxime/hydrazone formation reaction, free-radical polymerization reaction, or a combination thereof.

Moieties that participate in cycloaddition reactions may be utilized as attachment moieties. In cycloaddition reactions, two or more unsaturated moieties form a cyclic product with a reduction in the degree of unsaturation, these reaction partners are typically absent from natural systems, and so the use of cycloadditions for conjugation utilizes the introduction of unnatural functionality within a coupling partner. Exemplary moieties and their attachment reactions include:

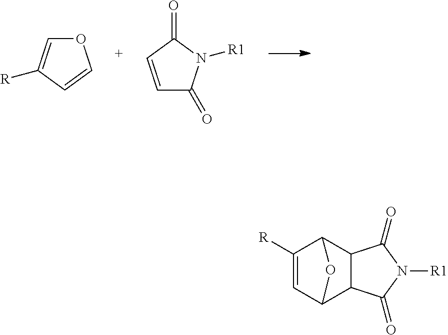

In some cases, moieties that participate in Copper-Catalyzed Azide-Alkyne Cycloadditions (CuAAC) may be utilized as attachment moieties. Optionally, moieties that participate in Strain-Promoted Azide-Alkyne Cycloadditions (SPAAC) may be utilized as attachment moieties. One of an azide or alkyne can be connected to an antibody conjugate and reacted with the other. A CuAAC or SPAAC reaction can be performed to produce a triazole attachment of the antibody conjugate to an object, substance or moiety.

Moieties that participate in inverse-electron demand Diels-Alder (IEDDA) reactions may be utilized as attachment moieties. One of a 1,2,4,5-tetrazine moiety, strained alkene moiety or strained alkyne can be connected to an antibody conjugate and, optionally, subjected to an IEDDA reaction. Exemplary moieties include, but are not limited to, trans-cyclooctenes, functionalized norbornene derivatives, triazines, or spirohexene. In some cases, a maleimide or furan can be used as an attachment moiety and, optionally, used in a hetero-Diels-Alder cycloaddition between a maleimide and furan. In some cases, a Diels-Alder reaction can achieve covalent coupling of a diene moiety with an alkene moiety to form a six-membered ring complex for attachment.

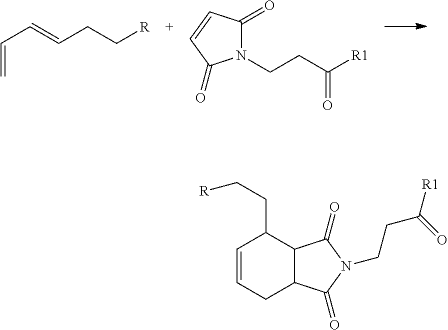

Accordingly, a diene moiety or alkene moiety can be used as an attachment moiety in an antibody conjugate. An oxime or hydrazone may be utilized as an attachment moiety. For example, attachment can be achieved via difunctional cross-linking.

Amines can be used in a variety of attachment reactions, such as those set forth below. Accordingly, an amine or other reactive moiety that participates in an attachment reaction can be used as an attachment moiety. An amine can react with an aldehyde via reductive amination to form an amine attachment. An isothiocyanate moiety may react with an amine to form a thiourea bond.

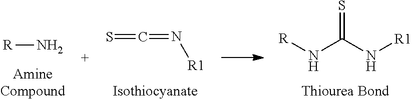

An isocyanate moiety can react with an amine moiety to form an isourea bond.

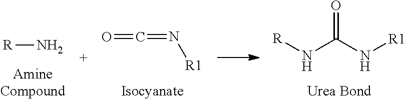

An acyl azide moiety can react with a primary amine to form an amide bond.
An N-hydroxysuccinimide (NHS) ester moiety can react with an amine moiety to form an amide bond.

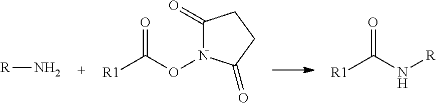

A sulfonyl chloride moiety can react with a primary amine to form a sulfonamide linkage.
An aryl halide moiety, such as fluorobenzene derivative, can form a covalent bond with an amine moiety. Other nucleophiles such as thiol, imidazolyl, and phenolate groups can also react with an aryl halide to form stable bonds. As shown below, a fluorobenzene moiety can react with an amine to form a substituted aryl amine bond.
An imidoester moiety can react with an amine to form an amidine linkage.

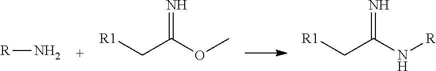

An epoxide or oxirane moiety can react with a nucleophile in a ring-opening process. The reaction can take place with primary amines, sulfhydryls, or hydroxyl groups to create secondary amine, thioether, or ether bonds, respectively.

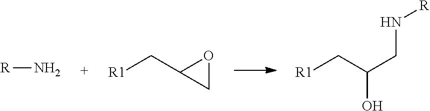

A carbonate moiety can react with a nucleophile to form a carbamate linkage.

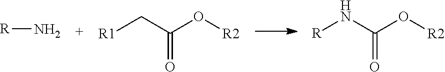

A carbonyl moiety, such as an aldehyde, ketone, or glyoxal, can react with an amine to form a Schiff base intermediate. In some cases, the addition of sodium borohydride or sodium cyanoborohydride will result in reduction of the Schiff base intermediate and covalent bond formation, creating a secondary amine bond.
A carboxylate moiety can be used as an attachment moiety. For example, diazomethane and other diazoalkyl derivatives may be reacted with carboxylate groups. In some cases, N,N′-Carbonyl diimidazole (CDI) may be used to react with a carboxylate to form N-acylimidazole which can then react with an amine to form an amide bond or with a hydroxyl to form an ester linkage.

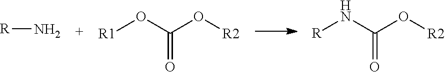

An N,N′-disuccinimidyl carbonate moiety or N-hydroxysuccinimidyl chloroformate moiety are reactive toward nucleophiles. These moieties can react with amines or hydroxyls to form stable crosslinked products.
A fluorophenyl ester moiety can react with an amine to form an amide bond. Exemplary fluorophenyl esters include pentafluorophenyl (PFP) ester, tetrafluorophenyl (TFP) ester, or sulfo-tetrafluoro-phenyl (STP) ester.

A tosylate ester can be used as an attachment moiety. For example, a tosylate ester can be formed from reaction of 4-toluenesulfonyl chloride (also called tosyl chloride or TsCl) with a hydroxyl moiety to yield a sulfonyl ester derivative. The sulfonyl ester can couple with a nucleophile to produce a covalent bond, for example, producing a secondary amine linkage with a primary amine, a thioether linkage with a sulfhydryl, or an ether bond with a hydroxyl.

Carbodiimide chemistry can be useful. Generally, carbodiimides are zero-length crosslinking agents that may be used to mediate the formation of an amide or phosphoramidate linkage between a carboxylate and an amine or between a phosphate and an amine, respectively. Accordingly, an amine, carboxylate, or phosphate can be used as an attachment moiety.

A hydroxymethyl phosphine moiety may be utilized as an attachment moiety. For example, tris(hydroxymethyl) phosphine (THP) and β-[tris(hydroxymethyl)phosphino] propionic acid (THPP) can react with nucleophiles, such as amines, to form covalent linkages.

An aldehyde or ketone moiety can be utilized as an attachment moiety. For example, derivatives of hydrazine, especially the hydrazide compounds formed from carboxylate groups, can react with aldehyde or ketone moieties. In some cases, a carboxylate moiety can be used as an attachment moiety.

An aminooxy can be utilized as an attachment moiety. For example, reaction can occur between an aldehyde and an aminooxy to yield an oxime linkage (aldoxime). This reaction is also quite efficient with ketones to form an oxime called a ketoxime. Accordingly, an aldehyde or ketone moiety can be used as an attachment moiety.

An attachment moiety can be photoreactive. For example, an aryl azide or halogenated aryl azide can be used. Their photoreactions are orthogonal to photo thiol-yne reactions because they are known to occur only under conditions much harsher than photo thiol-yne conditions such as by irradiation at short wavelength ultraviolet (UVC) for an extended period. Other photoreactive moieties are orthogonal to photo thiol-yne reactions and can be useful such as benzophenone.

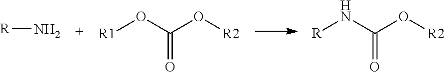

Further examples of useful photoreactive moieties include, for example, anthraquinone, alkyne, alkene, propargyl-ether, or propargyl-amine. Photocages can also be used as attachment moieties. Exemplary photocages are based around o-nitrobenzyl and coumarin chromophores. Photoaffinity moieties can be utilized and can achieve attachment via light induced formation of highly reactive intermediates, which then react with the nearest accessible functional group with high spatial precision. Examples include phenylazides, benzophenones, and phenyl-diazirines.

In some cases, a thiol or thiol-reactive moiety can be used as an attachment moiety. Generally, the attachment reaction can be configured to be orthogonal to a reaction used to modify cysteines of an antibody when making an antibody conjugate. In other cases, reactants used for attachment of an antibody conjugate to an object, substance or moiety can be blocked or absent when the antibody conjugate is formed. Conversely, reactants used for making an antibody conjugate can be blocked or absent when attachment of an antibody conjugate to an object, substance or moiety occurs. In other cases, selective conditions can be used. For example, selective modification of sulfhydryls can be achieved through careful control of pH, or redox state. For example, the lower pKₐ of aromatic thiols, when compared to their aliphatic counterparts can be exploited. Exemplary attachment moieties include sulfhydryls and moieties they react with in accordance with the reactions set forth below or elsewhere herein.

Activated halogen derivatives can be used to create sulfhydryl-reactive moieties such as haloacetyl, benzyl halides, and alkyl halides. In each of these compounds, the halogen group may be easily displaced by an attacking nucleophilic substance to form an alkylated derivative, such as the thioether bond resulting from reaction of a sulfhydryl with an iodoacetyl derivative as shown below.

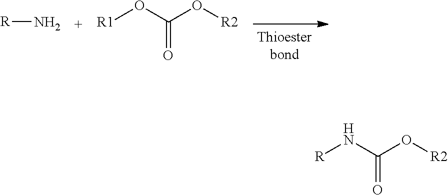

A maleimide moiety can undergo an alkylation reaction with a sulfhydryl to form a stable thioether bond.
An aziridine moiety can react with nucleophiles. For example, a sulfhydryls can react with aziridine moiety to form a thioether bond.
An acryloyl moiety can react with a sulfhydryl to form a thioether bond.
An electron-deficient aryl moiety can react with a sulfhydryl to form a substituted aryl bond.
A pyridyl dithiol moiety can undergo an interchange reaction with a free sulfhydryl to yield a mixed disulfide product.
A vinyl sulfone moiety can react with thiols, amines and hydroxyls. The product of the reaction of a thiol with a vinyl sulfone gives a beta-thiosulfonyl linkage.

An attachment moiety can be a binding moiety that includes an affinity reagent or functional derivative thereof. A binding moiety may bind with an analyte in a non-covalent manner. Some binding moieties can also be chemically reactive or catalytic (e.g., kinases, proteases, etc.). A binding moiety can be non-reactive and non-catalytic, thereby not permanently altering the chemical structure of an analyte to which it binds. An antibody, such as a full-length antibody or functional fragment thereof can form a binding moiety. Other useful affinity reagents that can form binding moieties include, for example, epitopes for antibodies, biotin, (strept)avidin, affibodies, affilins, affimers, affitins, alphabodies, anticalins, avimers, DARPins, monobodies, nanoCLAMPs, nucleic acids, polypeptides, nucleic acid aptamers, protein aptamers, lectins, carbohydrates or functional fragments thereof.

A nucleic acid may be utilized as an attachment moiety. For example, a nucleic acid attachment moiety can bind with high specificity to a complementary nucleic acid. Useful nucleic acids can have complementary sequences that are at least 5, 10, 15, 20, 25, 30, 40, 50, 60, 70, 80, 90 or more nucleotides in length. Alternatively or additionally, nucleic acids can have complementary sequences that are at most 90, 80, 70, 60, 50, 40, 30, 25, 20, 15, 10, 5 or fewer nucleotides in length. Accordingly, an object, substance or moiety that is to be attached to an antibody conjugate can include a nucleic acid having a sequence that is complementary to a sequence of a nucleic acid moiety of the antibody conjugate. Optionally, a nucleic acid that is used as an attachment moiety can be a nucleic acid origami or a component of a nucleic acid origami. For example, a nucleic acid moiety of an antibody conjugate can hybridize to a complementary sequence in an origami or other structured nucleic acid particle.

The attachment moieties and their reactions as set forth herein, such as those exemplified above, can be utilized with antibodies, proteins or other analytes.

A polypeptide can be used as a binding moiety. Useful binding sequences include, for example, at least 7, 8, 10, 12, 14, 16, 18 or 20 amino acids. Alternatively or additionally, a binding sequence can include at most 20, 18, 16, 14, 12, 10, 8 or 7 amino acids. Polypeptide moieties can include epitopes for an antibody or other affinity reagent set forth herein. Accordingly, an object, substance or moiety that is to be attached to an antibody conjugate can include an affinity reagent that recognizes a polypeptide attachment moiety of the antibody conjugate. In some cases, self-assembling polypeptides may be utilized for attachment. For example, polypeptides can adopt any of a variety of secondary structures, including β-sheets and α-helices, which can both stabilize individual sequences and facilitate interprotein aggregation. Optionally, non-natural functional groups can be appended to some or all of the amino acid monomers of a polypeptide moiety, for example, during polypeptide synthesis. A polypeptide moiety can include a known adhesion motif (e.g. RGD), laminin derived epitope, or growth factor mimetic that adheres to known polypeptide partners such as fibronectin. Polypeptides that form coiled coils, such as leucine zipper motifs can also be used.

An antibody conjugate of the present disclosure can include a label moiety connected to the antibody conjugate via a pair of synthetically bridged cysteines. For example, one or more of R¹, R², R³, and R⁴ of Formula I can be connected to a label moiety. A wide variety of label moieties may be useful including, for example, optically detectable labels, such as luminophores (e.g. fluorophores), enzymes (e.g. enzymes which catalyze reactions with colored reagents or products), electrochemical labels (e.g. highly charged moieties). Magnetic contrast imaging moieties can be used such as gadolinium-diethylenetriaminepentacetate (Gd-DTPA), gadolinium-dodecane tetraacetic acid (Gd-DOTA) or others used in magnetic resonance imaging (MRI) or other medical imaging devices. Moieties that are detected through subsequent processing, such as nucleic acid barcode labels, can also be useful. Nucleic acids can be detected or identified through nucleic acid amplification processes, sequencing processes or hybridization assays.

Any of a variety of luminophores may be used herein. In some cases, the luminophore may be a small molecule. In some cases, the luminophore may be a protein. Luminophores may include labels that emit in the ultraviolet, visible, or infrared region of the spectrum. In some cases, the luminophore may be selected from the group consisting of FITC, Alexa Fluor® 350, Alexa Fluor® 405, Alexa Fluor® 488, Alexa Fluor® 532, Alexa Fluor® 546, Alexa Fluor® 555, Alexa Fluor® 568, Alexa Fluor® 594, Alexa Fluor® 647, Alexa Fluor® 680, Alexa Fluor® 750, Pacific Blue, Coumarin, BODIPY FL, Pacific Green, Oregon Green, Cy3, Cy5, Pacific Orange, TRITC, Texas Red, R-Phycoerythrin, and Allophcocyanin (APC). In some cases, the label may be an Atto dye, for example Atto 390, Atto 425, Atto 430, Atto 465, Atto 488, Atto 490, Atto 495, Atto 514, Atto 520, Atto 532, Atto 540, Atto 550, Atto 565, Atto 580, Atto 590, Atto 594, Atto 610, Atto 611, Atto 612, Atto 620, Atto 633, Atto 635, Atto 647, Atto 655, Atto 680, Atto 700, Atto 725, Atto 740, Atto MB2, Atto Oxa12, Atto Rhol01, Atto Rho12, Atto Rho13, Atto Rho14, Atto Rho3B, Atto Rho6G, or Atto Thio12. In some cases, the luminophore may be a fluorescent protein such as green fluorescent protein (GFP), cyan fluorescent protein (CFP), red fluorescent protein (RFP), blue fluorescent protein (BFP), orange fluorescent protein (OFP), and yellow fluorescent protein (YFP). A wide range of effective luminophores are commercially available, for example, from the Molecular Probes division of ThermoFisher Scientific and/or generally described in the Molecular Probes Handbook (11th Edition) which is hereby incorporated by reference. Label components may also include intercalation dyes, such as ethidium bromide, propidium bromide, crystal violet, 4′,6-diamidino-2-phenylindole (DAPI), 7-aminoactinomycin D (7-AAD), Hoescht 33258, Hoescht 33342, Hoescht 34580, YOYO-1, DiYO-1, TOTO-1, DiTO-1, or combinations thereof.

The labels, methods of attachment and methods of detection set forth herein, such as those exemplified above, can be utilized with antibodies, proteins or other analytes.

An antibody conjugate of the present disclosure can be connected to a particle via a pair of synthetically bridged cysteines. For example, one or more of R¹, R², R³, and R⁴ of Formula I can be connected to a particle. Any of a variety of particles can be used. A particle may be composed of a natural, artificial, or synthetic material. For example, a particle can be composed of a nucleic acid such as a structured nucleic acid particle (SNAP), nucleic acid nanoball or nucleic acid origami; protein nucleic acid; protein; synthetic polymer; polysaccharide; organic particle; inorganic particle; gel such as hydrogel; metal; semiconductors; ceramic, glass, silica or the like. A particle can optionally include material having a polymeric structure. Alternatively, a particle need not include a polymeric structure.

A particle to which an antibody conjugate is attached can include one or more label moieties. For example, a particle can include at least 1, 2, 3, 4, 5, 10, 15, 20 or more label moieties. Alternatively or additionally, a particle can include at most 20, 15, 10, 5, 4, 3, 2, or 1 label moieties. For particles having multiple label moieties, the label moieties can have the same structure or they can differ structurally from each other. Optionally, label moieties on a given particle can have the same detectable characteristics or they can be distinguishable from each other. For example, two or more optical labels can differ in terms of luminescence lifetime, optical polarity, spectral region for absorbance, spectral region excitation or spectral region emission. Alternatively, two or more optical labels can be indistinguishable in terms of luminescence lifetime, optical polarity, spectral region for absorbance, spectral region excitation or spectral region emission. This can result from the two or more optical labels having the same structure, but in some cases two or more labels can have the same or overlapping detection properties despite having different structures. Particularly useful particles for accommodating multiple label moieties include, but are not limited to, structured nucleic acid particles. Exemplary particles having one or more label moieties and which can be modified in accordance with the present disclosure are set forth in US Pat. App. Pub. No. 2022/0162684 A1, which is incorporated herein by reference.

In some configurations, a particle can be attached to a plurality of antibody conjugates. For example, a particle can include at least 1, 2, 3, 4, 5, 10, 15, 20 or more antibody conjugates. Alternatively or additionally, a particle can include at most 20, 15, 10, 5, 4, 3, 2, or 1 antibody conjugates. Multiple antibody conjugates that are attached to a given particle can have the same or different function compared to each other. For example, multiple antibody conjugates attached to a particle can have affinity for the same epitope or set of epitopes. In some cases, the affinity can be substantially the same, for example, in cases where the antibody conjugates have the same structure. In other cases, the affinity of two or more antibody conjugates for a given epitope or set of epitopes can overlap despite some differences in the functional or structural characteristics of the two or more antibody conjugates. In some configurations, multiple antibody conjugates that are attached to a given particle can have affinity for different epitopes. This can be the case, whether the different epitopes are found in the same analyte (e.g. two different amino acid trimer epitopes present in a given protein analyte) or different analytes (e.g. a first trimer epitope being found in a first protein that lack a second trimer epitope, and a second trimer epitope being found in a second protein that lacks the first trimer epitope). Particularly useful particles for accommodating multiple antibody conjugates include, but are not limited to, structured nucleic acid particles. Exemplary particles having one or more attached affinity reagents and which can be modified in accordance with the present disclosure are set forth in US Pat. App. Pub. No. 2022/0162684 A1, which is incorporated herein by reference.

Nucleic acid origami provides a particularly useful material for a particle. Accordingly, a particle can include one or more nucleic acids having tertiary or quaternary structures such as spheres, cages, tubules, boxes, triangles, icosahedrons, tiles, blocks, trees, pyramids, wheels or combinations thereof. Examples of such structures formed with DNA origami are set forth in Zhao et al. Nano Lett. 11, 2997-3002 (2011); Rothemund Nature 440:297-302 (2006); Sigle et al, Nature Materials 20:1281-1289 (2021); or U.S. Pat. Nos. 8,501,923 or 9,340,416, each of which is incorporated herein by reference. In some configurations, a nucleic acid origami may include a scaffold and a plurality of staples. The scaffold can be configured as a single, continuous strand of nucleic acid, and the staples can be formed by oligonucleotides that hybridize, in whole or in part, with the scaffold nucleic acid. A particle including one or more nucleic acids (e.g. as found in origami or nanoball structures) may include regions of single-stranded nucleic acid, regions of double-stranded nucleic acid, or combinations thereof.

In some configurations, a nucleic acid origami includes a scaffold composed of a nucleic acid strand to which a plurality of oligonucleotides is hybridized. A nucleic acid origami may have a single scaffold molecule or multiple scaffold molecules. A scaffold nucleic acid can be linear (i.e. having a 3′ end and 5′ end) or circular (i.e. closed such that the scaffold lacks a 3′ end and 5′ end). A nucleic acid scaffold can be derived from a natural source, such as a viral genome or a bacterial plasmid. For example, a nucleic acid scaffold can include a single strand of an M13 viral genome. In other configurations, a nucleic acid scaffold may be synthetic, for example, having a non-naturally occurring sequence in full or in part. A scaffold nucleic acid can be single stranded but for a plurality of oligonucleotides hybridized thereto or short regions of internal complementarity. The size of a nucleic acid scaffold may vary to accommodate different uses. For example, a nucleic acid scaffold may include at least about 100, 500, 1000, 2500, 5000, 10000, 50000 or more nucleotides. Alternatively or additionally, a nucleic acid scaffold may include at most about 50000, 10000, 5000, 2500, 1000, 500, 100 or fewer nucleotides.

A nucleic acid origami can include a plurality of oligonucleotides that are hybridized to a scaffold nucleic acid. A first region of an oligonucleotide sequence can be hybridized to a scaffold nucleic acid while a second region is not hybridized to the scaffold. The second region can be in a single stranded state or, alternatively, can participate in a hairpin or other self-annealed structure in the oligonucleotide. In some cases, the second region of the oligonucleotide can hybridize to a complementary oligonucleotide to form a double-stranded region. An oligonucleotide can include two sequence regions that are hybridized to a scaffold nucleic acid, for example, to function as a ‘staple’ that restrains the structure of the scaffold. For example, a single oligonucleotide can hybridize to two regions of a scaffold that are separated from each other in the primary sequence of the scaffold. As such, the oligonucleotide can function to retain those two regions of the scaffold in proximity to each other or to otherwise constrain the scaffold to a desired conformation. Two sequence regions of an oligonucleotide staple can be adjacent to each other in the oligonucleotide sequence or separated by a third region that does not hybridize to the scaffold. One or more regions of an oligonucleotide that hybridize to a scaffold nucleic acid can be located at or near the 5′ end of the oligonucleotide, at or near the 3′ end of the oligonucleotide, or in a region of the oligonucleotide that is between the end regions. Oligonucleotides can be configured to hybridize with a nucleic acid scaffold, another oligonucleotide, a staple oligonucleotide, or a combination thereof. The oligonucleotides can be linear (i.e. having a 3′ end and a 5′ end) or closed (i.e. circular, lacking both 3′ and 5′ ends).

An oligonucleotide that is included in a nucleic acid origami can have any of a variety of lengths. An oligonucleotide may have a length of at least about 10, 25, 50, 100, 250, 500, or more nucleotides. Alternatively or additionally, an oligonucleotide may have a length of no more than about 500, 250, 100, 50, 25, 10, or fewer nucleotides. An oligonucleotide in a nucleic acid origami may hybridize with another oligonucleotide or a scaffold strand forming a particular number of base pairs. An oligonucleotide may form a hybridization region of at least about 5, 6, 7, 8, 9, 10, 15, 20, 25, 50 or more consecutive or total base pairs. Alternatively or additionally, an oligonucleotide may form a hybridization region of no more than about 50, 25, 20, 15, 10, 9, 8, 7, 6, 5, or fewer consecutive or total base pairs.

An antibody conjugate, label moiety or other moiety can be attached to nucleic acid origami via a scaffold component or oligonucleotide component of the origami structure. For example, the scaffold or oligonucleotide can include a nucleotide analog that attaches covalently or non-covalently to the antibody conjugate, label moiety or other moiety. Examples of structured nucleic acid particles that include nucleic acid origami are set forth, for example, in U.S. Pat. No. 11,203,612; US Pat. App. Pub. No. 2022/0162684 A1 or U.S. patent application Ser. No. 17/692,035, each of which is incorporated herein by reference.

A structured nucleic acid particle (e.g., nucleic acid origami, or nucleic acid nanoball) may be formed by an appropriate technique including, for example, those known in the art. Nucleic acid origami may be fabricated, for example, using techniques described in Rothemund, Nature 440:297-302 (2006), or U.S. Pat. Nos. 8,501,923 or 9,340,416, each of which is incorporated herein by reference. Nucleic acid origami may be designed using a software package, such as CADNANO (cadnano.org), ATHENA (github.com/lcbb/athena), or DAEDALUS (daedalus-dna-origami.org). Nucleic acid nanoballs may be fabricated by a method such as rolling circle amplification using a circular template to generate a nucleic acid amplicon consisting of a concatemer of template complements. The amplicon can be further modified to include crosslinks, for example, in the form of staples that hybridize to different regions of the amplicon. Exemplary methods for making nucleic acid nanoballs are described, for example, in U.S. Pat. No. 8,445,194, which is incorporated herein by reference.

A particle need not be composed primarily of nucleic acid and, in some cases, may be devoid of nucleic acids. For example, a particle can be composed of a solid support material, such as a silicon or silica nanoparticle, a carbon nanoparticle, a cellulose nanobead, a PEG nanobead, a polymeric nanoparticle (e.g., polyethyleneimine, dendritic polymer, dendrimer, polyacrylate particle, polystyrene-based particle, FluoSphere™, etc.), upconversion nanocrystal, or a quantum dot. A particle may include solid materials and shell-like materials (e.g., carbon nanospheres, silicon oxide nanoshells, iron oxide nanospheres, polymethylmethacrylate nanospheres, etc.). A particle may include distinct surfaces, such as plates or shells. In some configurations, a particle may include a gel material.

A particle may have any of a variety of sizes and shapes to accommodate use in a desired application. For example, a particle can have a regular or symmetric shape or, alternatively, a particle can have an irregular or asymmetric shape. The shape can be rigid or pliable. The size or shape of a particle can be characterized with respect to length, area, or volume. The length, area or volume can be characterized in terms of a minimum, maximum, or average for a population. Optionally, a particle can have a minimum, maximum or average length of at least about 50 nm, 100 nm, 250 nm, 500 nm, 1000 nm or more. Alternatively or additionally, a particle can have a minimum, maximum or average length of no more than about 1000 nm, 500 nm, 250 nm, 100 nm, 51 nm, or less. Optionally, a particle can have a minimum, maximum or average volume of at least about 1 μm³, 10 μm³, 100 μm³, 1 mm³, 10 mm³, 100 mm³, 1 cm³ or more. Alternatively or additionally, a particle can have a minimum, maximum or average volume of no more than about 1 cm³, 100 mm³, 10 mm³, 1 mm³, 100 μm³, 10 μm³, 1 μm³ or less.

A particle can be characterized with respect to its footprint (e.g. occupied area on a surface). A footprint may have a regular shape or an approximately regular shape, such as triangular, square, rectangular, circular, ovoid, or polygon. Optionally, the minimum, maximum or average area for a particle footprint can be at least about 10 nm², 100 nm², 1 μm², 10 μm², 100 μm², 1 mm² or more. Alternatively or additionally, the minimum, maximum or average area for a particle footprint can be at most about 1 mm², 100 μm², 10 μm², 1 μm², 100 nm², 10 nm², or less.

A particle that is used in accordance with the present disclosure can be suspended in a fluid, immobilized on a solid support, or immobilized in another material such as a solid support material. For example, a population of particles can be colloidal for some, or all steps of a method set forth herein. Alternatively, a population of particles can be immobilized in, or on a solid support, for example, by gravity, non-covalent bonding, covalent bonding, coordination, adhesion or a combination thereof. Optionally, a first particle can be attached to a second particle. The first and second particles can compose the same material as each other or different materials from each other.

The particles, particle attachment methods and methods for making particle-analyte conjugates as set forth herein, such as those exemplified above, can be utilized with antibodies, proteins or other analytes.

An antibody conjugate of the present disclosure can be connected to a solid support via a pair of synthetically bridged cysteines. For example, one or more of R¹, R², R³, and R⁴ of Formula I can be connected to a solid support. The solid support can be made of a material set forth herein in the context of particles. In some cases, the solid support has an array of addresses that are competent for attachment of antibody conjugates, or attachment of a plurality of antibody conjugate forms an array of antibody conjugates.

Accordingly, the present disclosure provides an array including a plurality of addresses and a plurality of antibodies, wherein each of the antibodies includes a pair of cysteines crosslinked via a two-carbon bridge covalently connecting the sulfur atoms of the cysteines, and wherein the two-carbon bridge further comprises a linker attached to an address of the plurality of addresses. For example, the array can include a plurality of addresses and a plurality of antibodies, wherein individual addresses of the plurality of addresses are each attached to an antibody of the plurality of antibodies, wherein each of the individual addresses is attached to a pair of modified cysteines of the antibody, the modified cysteines having a moiety of Formula I:

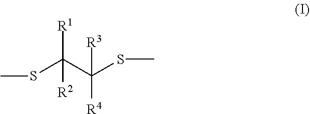

wherein S represents the sulfur moieties of the cysteines, wherein R¹, R², R³, and R⁴ are each independently selected from any of a variety of atoms or organic moieties, and wherein at least one of R¹, R², R³, and R⁴ comprises a linker connecting an antibody of the plurality of antibodies to an individual addresses of the plurality of addresses. Optionally, R¹, R², R³, and R⁴ are each independently selected from the group consisting of hydrogen, halo, alkyl, alkenyl, alkynyl, aryl, heteroalkyl, heteroalkenyl, heteroalkynyl, heteroaryl, and optionally substituted variants thereof.

In some configurations, the array is configured for single-molecule resolution. For example, individual addresses of the array can each be attached to no more than one antibody conjugate or no more than one modifying reagent. Alternatively, individual addresses of the array can each be attached to an ensemble of antibody conjugates or an ensemble of modifying reagents.

Optionally, attachment of an antibody conjugate or modifying reagent to an address is mediated by a particle, such as a structured nucleic acid particle or other particle set forth herein. Alternatively, attachment need not be mediated by any particle, or at least not by one or more of the particles set forth herein. In particular configurations, at least one of R1, R2, R3, and R4 of Formula I can connect an antibody conjugate to an address of an array, for example, via a particle or absent a particle.

The addresses of an array can optionally be optically observable and, in some configurations, adjacent addresses can be optically distinguishable when detected. Addresses of an array are typically discrete. The discrete addresses can be contiguous, or they can have interstitial spaces between each other. An array useful herein can have, for example, addresses that are separated by less than 100 microns, 10 microns, 1 micron, 500 nm, 100 nm, 10 nm or less. Alternatively or additionally, an array can have addresses that are separated by at least 10 nm, 100 nm, 500 nm, 1 micron, 5 microns, 10 microns, 50 microns, 100 microns or more. The addresses can each have an area of less than 1 square millimeter, 500 square microns, 100 square microns, 25 square microns, 1 square micron or less. An array can include at least about 1×10⁴, 1×10⁵, 1×10⁶, 1×10⁸, 1×10¹⁰, 1×10¹², or more addresses.

An antibody conjugate of the present disclosure can include a linker connecting a pair of synthetically bridged cysteines to an object, substance or moiety. In particular configurations, the pair of cysteines is crosslinked via a two-carbon bridge and the two-carbon bridge is connected to an object, substance or moiety via the linker. As such, an antibody of the present disclosure can include a tripartite linkage including first and second bonds to the sulfurs of a pair of cysteines of the antibody and a third bond (covalent or non-covalent) to an object, substance or moiety.

A linker can contain a polymer including, but not limited to, a synthetic polymer such as polyethylene glycol (PEG), polyethylene oxide (PEO), polyethyleneimine (PEI), an alkane chain, nucleic acid origami, polyamido amine (PAMAM) dendrimer, polylysine dendrimer, a broad range of dendrimers, or protein nucleic acid (PNA), or biological polymer such as a polypeptide (i.e. protein), nucleic acid (e.g. DNA or RNA), or polysaccharide. Synthetic analogs of biological polymers can also be useful. A polymeric linker can include at least 1, 2, 3, 4, 5, 10 or more monomer units. Alternatively or additionally, a polymeric linker can include at most 10, 5, 4, 3, 2 or 1 monomer units. The monomer units can be, for example, ethylene glycol monomers, ethylene oxide monomers, amino acid monomers, nucleotide monomers, saccharide monomers or other monomers present in a polymer set forth herein. A linker can be devoid of one or more of the polymers, monomers or other moieties set forth above or elsewhere herein.

A linker moiety can be connected to an alkyne or alkene moiety via a spacer moiety. Alternatively or additionally, a polymer or other linker moiety can be connected to an object, substance or moiety via a spacer moiety. Spacer moieties can include, for example, moieties resulting from synthetic attachment of the linker moiety to another moiety. A linker moiety or spacer moiety can contain at least one amino acid, nucleotide, saccharide, amide, carboxyl, amine, alcohol, or halogen. Useful linkers and spacers are set forth in U.S. Pat. No. 9,951,141, which is incorporated herein by reference.

A linker can be a rigid linker or a flexible linker. Exemplary flexible linkers include, but are not limited to, PEG, PEO, PEI, polypeptide, carbohydrate, alkane chain, single stranded nucleic acid, or combination thereof. A double-stranded nucleic acid or branched alkane chain can be used as a rigid linker. A polypeptide linker can be engineered for rigidity or flexibility by appropriate choice of amino acid constituents. See, for example, Chen et al., Adv Drug Deliv Rev. 65(10):1357-1369 (2013) and Argos J Mol Biol. 211:943-958 (1990), each of which is incorporated herein by reference.

A linker can be cleavable or non-cleavable under conditions of use. The conditions of use can include, for example, in vivo, in vitro, or in an assay set forth herein. By way of example, a nucleic acid linker can be cleavable due to presence of a restriction site, non-natural nucleotide (e.g. a uracil or 8-oxoguanine nucleotide in DNA), or other enzymatically or chemically cleavable site. A polypeptide linker can be cleavable due to presence of a protease site or chemically susceptible bond in the polypeptide backbone. A cleavable linker can include a photocleavable moiety such that the linker can be photolyzed. A non-cleavable linker can lack one or more of the cleavable sites exemplified above.

The solid supports, methods for attachment to solid supports, linkers, and methods for using solid supports, such as those exemplified above, can be utilized with antibodies, proteins or other analytes.

The present disclosure provides a method of modifying an antibody by reacting an alkyne moiety of a modifying reagent with the sulfhydryls of a pair of cysteines, thereby forming a two-carbon bridge between the sulfhydryls of the pair of cysteines. For example, the method can include reacting cysteines of an antibody, with an alkyne moiety of a modifying reagent, wherein the alkyne moiety reacts with the cysteines to form a moiety of Formula I:

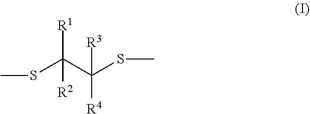

wherein S represents the sulfur moieties of the cysteines, and wherein R¹, R², R³, and R⁴ are each independently selected from any of a variety of atoms or organic moieties. Optionally, R¹, R², R³, and R⁴ are each independently selected from the group consisting of hydrogen, halo, alkyl, alkenyl, alkynyl, aryl, heteroalkyl, heteroalkenyl, heteroalkynyl, heteroaryl, and optionally substituted variants thereof.

In particular configurations, reacting cysteines of an antibody, with an alkyne moiety of a modifying reagent can be carried out under irradiation with light to drive formation of a two-carbon bridge between the cysteines, such as the bridge of Formula I. Photocatalysts such as azo-based compounds can be used to generate radical species under irradiation conditions. Exemplary photocatalysts include, but are not limited to, 2,2-azobis(isobutyronitrile) (AIBN),azobis(2-methylpropionamidine) dihydrochloride (AAPH) and lithium phenyl-2,4,6-trimethylbenzoylphosphinate (LAP). Without intending to be limited by mechanism, the reaction can be, at least in some configurations of the methods set forth herein, a photo thiol-yne reaction. Light can be provided at a wavelength or wavelength range that is compatible with the reaction components. For example, the light can be in the UV range of the spectrum (10 nm to 400 nm). In some cases, it may be desirable to use light in different sub-regions of the UV including, for example, the long wavelength ultraviolet (UVA) range (315 nm-400 nm), medium wavelength ultraviolet (UVB) range (280-315), or short wavelength ultraviolet (UVC) range (200-280 nm). Light in the longer wavelengths, for example, in the visible (VIS) or near infrared (NIR) regions of the spectrum can be used, for example, to improve light penetration and absorption by photocatalysts such as azobis(2-methylpropionamidine) dihydrochloride (AAPH) or lithium phenyl-2,4,6-trimethylbenzoylphosphinate (LAP), or to minimize damage to antibodies or other components that are present in the reaction. Alternatively, light can be delivered at the lower wavelengths or middle wavelengths of the range to achieve higher reaction yield. Although delivery of light at wavelengths below 400 nm, 315 nm or 280 nm can be desirable, it may also be useful to deliver light at higher wavelengths, for example, in the visible region of the spectrum such as wavelengths at or below the red, orange, yellow, green, blue, or violet regions of the visible spectrum. This can be achieved using a mechanism of two-photon absorption or by adding upconversion nanocrystals which are emitting UV-VIS bands of light upon light excitation at a near infrared region (e.g. 808 nm, 980 nm). The light that is delivered in a reaction between cysteines and an alkyne moiety can exclude wavelengths in a particular range of the spectrum including, but not limited to, one or more of the ranges set forth herein.

Light can be delivered for a period of time that is suitable to achieve a desired yield of antibody conjugate. For example, light can be delivered for at least 1, 2, 5, 10, 15, or 30 minute(s). Alternatively or additionally, it may be desirable to deliver light for at most 30, 15, 10, 5, 2 or 1 minute(s). The radiant flux of the light can be, for example, 8 watts, 10 watts, 100 watts, 200 watts, 300 watts or more. In some cases lower wattage can be used.

The use of light to drive formation of a two-carbon cysteine bridge, such as the bridge of Formula I, can be particularly useful when the alkyne moiety is not present in a strained ring. For example, the alkyne moiety can be in a linear hydrocarbon or in a ring having at least 6, 7, 8 or more members. However, a method for making an antibody conjugate can include delivery of light to a reaction that occurs between a pair of cysteines and an alkyne moiety that is present in a strained ring. A method of modifying an antibody to produce a pair of cysteines having a two-carbon bridge, such as Formula I, can employ a modifying reagent having a cyclooctyne, cycloheptyne, cyclohexyne or cyclopentyne moiety. A dibenzocyclooctyne moiety can be particularly useful. Other cyclooctyne moieties can be particularly useful such as those derived from 3,3-difluoro-substituted cyclo-1-octyne and cyclopropane-fused bicyclo[6.1.0]nonyne.

Light can be delivered to a sample in a spatially resolved way to produce photoproducts at spatially resolved locations. For example, a tissue sample can be in contact with reagents having an alkyne moiety that is relatively inert, for example not being present in a strained ring, and a localized area of the tissue can be irradiated with light to produce a photoproduct set forth herein. Areas of the tissue that are not irradiated with light will not substantially react with the alkyne even if sulfur moieties are present in those areas of the tissue. An area of a tissue to be irradiated can be selected based on any of a variety of criteria including, but not limited to, a random sampling, an area that appears to be affected by a particular condition or disease such as cancer, an area that appears to be non-affected by a particular condition or disease such as cancer, an area that has been treated with a therapeutic agent or candidate therapeutic agent, an area that has not been treated with a therapeutic agent or candidate therapeutic agent, an area that has been subjected to a particular experimental condition or reagent, or an area that has not been subjected to a particular experimental condition or reagent. Results for irradiated areas of a tissue can be compared with results for non-irradiated areas of the tissue (or non-irradiated area of a control tissue) to better understand a characteristic of the tissue or a component of the tissue (e.g. cells, proteins, nucleic acids or other components). Other samples to which light can be delivered in spatially resolved way for spatially resolved photoreactions include, but are not limited to, proteins or other analytes attached to solid supports such as arrays wherein the proteins or other analytes are attached at respective addresses, particles or cells that are separated from each other in a solution or on a solid support such as a petri plate, or particles or cells that are separated from each other in a fluid stream such as a fluorescent activated cell sorter.

In some configurations of the methods set forth herein, light is not delivered to a reaction between cysteines and an alkyne moiety. For example, the reaction can be carried out in absence of light or under standard laboratory lighting that is not enriched for light having wavelengths at or below the UV range or other spectral range set forth herein. Reactions that are not activated by light can be carried out using an alkyne that is activated by other mechanisms, such as via ring strain. In some cases, an alkyne can be reacted with sulfhydryls at elevated temperatures such that radicals are formed thermally and stimulate formation of a sulfur cross link as set forth herein. Elevated temperatures can include temperatures above, 40° C., 45° C., 50° C., 60° C., 70° C. or higher. Alternatively or additionally, the temperature can be maintained below 70° C., 60° C., 50° C., 45° C., 40° C., or lower. For example, the temperature can be capped to maintain structural integrity and/or activity of a protein that is to be crosslinked or otherwise modified in a method set forth herein.

The photoreactions, reagents and conditions set forth herein, such as those exemplified above, can be utilized with antibodies, proteins or other analytes.

A method of the present disclosure can be carried out to produce an antibody conjugate having at least 1, 2, 3, 4 or more pair(s) of cysteines having a two-carbon bridge, such as Formula I. Alternatively or additionally, a method of the present disclosure can be carried out to produce an antibody conjugate having at most 4, 3, 2 or 1 pair(s) of cysteines having a two-carbon bridge, such as Formula I. The number of cysteine pairs that are modified to have a two-carbon bridge can be controlled, for example, via genetic engineering to add, remove or relocate cysteines in the antibody structure, via selective reduction of some cysteines compared to others in the antibody prior to reaction with a modifying reagent having an alkyne moiety, via selective oxidation of some cysteines compared to others in the antibody prior to reaction with a modifying reagent having an alkyne moiety, or via selective reaction of reduced cysteines with an alkyne moiety, or a combination thereof.

A method of modifying an antibody can be configured to react an alkyne moiety with a sulfur moiety on a heavy chain of the antibody and a sulfur moiety on a light chain of the antibody, thereby producing an antibody conjugate having a synthetic crosslink between the heavy chain and the light chain. Optionally, the method can further react an alkyne moiety with a sulfur moiety on a second heavy chain of the antibody and a sulfur moiety on a second light chain of the antibody, whereby the antibody conjugate has a synthetic cros slink between a first heavy chain and a first light chain and whereby the antibody conjugate has a synthetic crosslink between the second heavy chain and the second light chain. As an alternative or addition to modifying at least one cysteine pair to form synthetic crosslink(s) between heavy and light chain(s), a method of modifying an antibody can be configured to react an alkyne moiety with a sulfur moiety on a first heavy chain of the antibody and a sulfur moiety on a second heavy chain of the antibody, thereby producing an antibody conjugate having a synthetic crosslink between the first and second heavy chains. Optionally, the method can further react an alkyne moiety with a second sulfur moiety on the first heavy chain of the antibody and a second sulfur moiety on the second light chain of the antibody, whereby the antibody conjugate has two synthetic crosslinks between the first and second heavy chains.

Optionally, a method of modifying an antibody can be configured to react an alkyne moiety with a first sulfur moiety on a heavy chain of the antibody and a second sulfur moiety on the heavy chain of the antibody, thereby producing an antibody conjugate having a synthetic crosslink within the heavy chain. If desired, a method of modifying an antibody can be configured to react an alkyne moiety with a first sulfur moiety on a second heavy chain of the antibody and a second sulfur moiety on the second heavy chain of the antibody, thereby producing an antibody conjugate having a synthetic crosslink within the first heavy chain and another synthetic cros slink within the second heavy chain.

In many cases, an antibody that is to be modified in a method set forth herein, will contain pairs of cysteines that are bridged by a disulfide. These oxidized cysteines can be reduced prior to being reacted with an alkyne moiety to produce a two-carbon bridge between the cysteines. The antibody can be contacted with any of a variety of reducing agents including, for example, thioredoxin, dithiothreitol (DTT), 2-mercaptoethanol (2-ME), tris(2-carboxyethyl)phosphine (TCEP), tris(3-hydroxypropyl)phosphine) (THPP), 2-aminoethanethiol, glutathione, and 1,4-dithio-2-butylamine (DTBA).

In some configurations of the methods set forth herein, pairs of cysteines in different regions of an antibody are selectively reduced. Selective reduction of cysteines in an antibody followed by reaction of the selectively reduced antibody with a modifying reagent having an alkyne moiety can yield an antibody conjugate that contains one or more pairs of cysteines that are selectively modified to contain a two-carbon bridge, while at least one pair of cysteines in the antibody is not modified to have a two-carbon bridge. Optionally, one or two pairs of cysteines that form a disulfide bridge between a heavy chain and light chain can be selectively reduced while other pairs of cysteines are in an oxidized state. For example, disulfides between heavy and light chains can be preferentially reduced using less than 4 molar equivalents of a strong reducing agent such as DTT or TCEP compared to the quantity of antibody in the reaction (see Sun et al. Bioconjug Chem. 16(5):1282-1290 (2005), which is incorporated herein by reference). In an alternative option, one or two pairs of cysteines that form a disulfide bridge between heavy chains can be selectively reduced while other pairs of cysteines are in an oxidized state. For example, disulfides between heavy chains can be preferentially reduced by fully reducing all cysteine pairs in an antibody and then selectively re-oxidizing cysteines that form disulfides between heavy and light chains of the antibody. The selective reoxidation can be achieved by treatment of the fully reduced antibody with 2 to 3 molar equivalents of DTNB or 4,4′-dipyridyldithiol compared to the quantity of reduced antibody (see Sun et al. Bioconjug Chem. 16(5):1282-1290 (2005), which is incorporated herein by reference). Reduction and oxidation methods exemplified herein with regard to antibodies, such as those set forth above, can be applied to proteins or other analytes.

An antibody can be genetically engineered to add or remove cysteines prior to being modified in a method set forth herein. For example, one or more cysteines can be removed as a way to preclude unwanted modification of the antibody at the site of the removed cysteine(s). For example, an antibody can be engineered to omit at least 1, 2, 3 or 4 cysteines that would otherwise form a disulfide bridge between two heavy chains. Alternatively or additionally, an antibody can be engineered to omit at least 1, 2, 3 or 4 cysteines that would otherwise form a disulfide bridge between a heavy chain and light chain. Whether or not any cysteines are removed from an antibody, the antibody can be engineered to add one or more cysteines at one or more respective positions where cysteine(s) are not typically found in nature. An added cysteine can be modified to form a two-carbon bridge with another cysteine in the structure of the engineered antibody. In some configurations, a cysteine that has been added to an engineered antibody is modified in a method of the present disclosure to form a two-carbon bridge (e.g. a bridge having a structure of Formula I) with a native cysteine in the engineered antibody. Alternatively, a cysteine that has been added to an engineered antibody can be modified in a method of the present disclosure to form a two-carbon bridge with another added cysteine in the engineered antibody. One or more cysteines can be added to an engineered antibody to facilitate formation of a two-carbon bridge (e.g. a bridge having a structure of Formula I) between two heavy chains, between a heavy chain and a light chain, within the same heavy chain or within the same light chain. Optionally, one or more cysteines can be added to an engineered version of an antibody fragment. The added cysteines can form a two-carbon bridge (e.g. a bridge having a structure of Formula I) within a single chain of the engineered antibody fragment or between two chains of the engineered antibody fragment. Similar engineering techniques can be used to add or remove cysteines from other proteins that are to be used in a method set forth herein.

A modifying reagent that is used in a method set forth herein to produce an antibody conjugate having a pair of cysteines with a two-carbon bridge can include an attachment moiety. The attachment moiety of a modifying reagent having an alkyne moiety can be inert to reaction conditions used to react the alkyne moiety with a pair of cysteines to form a synthetic bridge between the cysteines of the pair. For example, the attachment moiety can have reactivity that is orthogonal to the reactivity of the alkyne or cysteines. In some cases, the attachment moiety is a binding moiety that is not chemically reactive with the antibody that is to be modified. In another option, the attachment moiety can be rendered temporarily inert by a blocking agent.

An antibody conjugate that includes a pair of cysteines with a two-carbon bridge (e.g. a bridge having a structure of Formula I) and an attachment moiety can be contacted with an object, substance or moiety that is reactive with the attachment moiety, thereby attaching the antibody conjugate to the object, substance or moiety. Taking the example of an attachment moiety that functions as a binding moiety, the object, substance or moiety can include a binding partner to the binding moiety. Exemplary binding partners include, but are not limited to, complementary nucleic acids, receptors and their ligands, affinity reagents (e.g. antibodies, aptamers, etc.) and their epitopes, self-assembling peptides, and (strept)avidin and biotin (or analogs of biotin). In other examples, the attachment moiety that is present in an antibody conjugate can be a moiety that is chemically reactive with a moiety on an object, substance or moiety to which the antibody conjugate is to be attached. Any of a variety of chemically reactive moieties can be used including for example those set forth herein or those known in the art for crosslinking of proteins and similar biomolecules. Any of a variety of objects, substances or moieties can be attached to an antibody conjugate including, for example, a particle (e.g. structured nucleic acid particle), solid support, protein, second antibody, nucleic acid, label or others known in the art or set forth herein.

A modifying reagent that is used in a method set forth herein to produce an antibody conjugate having a pair of cysteines with a two-carbon bridge (e.g. a bridge having a structure of Formula I) can include a label moiety, for example, a label moiety set forth herein. The label moiety can be convenient for assessing the yield of antibody conjugate produced in a method set forth herein. Optionally, the label moiety can be used to characterize an antibody conjugate to which it is attached. The label moiety of an antibody conjugate can facilitate detection of the conjugate in any of a variety of downstream applications including, for example, assay of proteins or other analytes that are recognized by the antibody conjugate, imaging of cells or tissues having epitopes of the antibody conjugate, or the like.

A modifying reagent that is used in a method set forth herein to produce an antibody conjugate having a pair of cysteines with a two-carbon bridge (e.g. a bridge having a structure of Formula I) can be attached to a solid support. Accordingly, in some configurations a method for modifying an antibody is carried out on a solid support to which the modifying reagent is attached. The solid support can have an array of addresses, individual addresses each being attached to a single modifying reagent or to a plurality of modifying reagents. Multiple addresses can be simultaneously reacted with antibody in a method set forth herein. For example, a solution containing a plurality of antibodies can be delivered to an array such that the antibodies are in diffusional contact with a plurality of modifying reagents at a plurality of sites on the array. Optionally, a modifying reagent that is used in a method set forth herein to produce an antibody conjugate can be attached to a particle such as a structured nucleic acid particle. The particles can be colloidal or located on a surface during the modification reaction.

A modifying reagent that is used in a method set forth herein to produce an antibody conjugate having a pair of cysteines with a two-carbon bridge can include a linker moiety. Exemplary linker moieties include those known in the art or set forth elsewhere herein. The modifying reagents and linkers exemplified herein for antibodies, such as those set forth above, can be used with proteins or other analytes.

The present disclosure provides an antibody, including a cysteine having a moiety of Formula II:

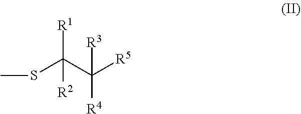

### or Formula III:

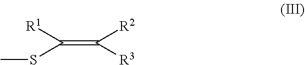

wherein S represents the sulfur moiety of the cysteine, and wherein R¹, R², R³, R⁴ and R⁵ are each independently selected from any of a variety of atoms or organic moieties. Optionally, R¹, R², R³, R⁴ and R⁵ are each independently selected from the group consisting of hydrogen, halo, alkyl, alkenyl, alkynyl, aryl, heteroalkyl, heteroalkenyl, heteroalkynyl, heteroaryl, and optionally substituted variants thereof.

In some configurations an antibody will include only a single cysteine that has a moiety of Formula II or Formula III. However, multiple cysteines can be modified. For example, an antibody can include any combination of cysteines having a structure of Formula II or Formula III. In some configurations, one or more cysteines having a structure of Formula II or Formula III can be present in a single HC, two HCs, a single LC, two LCs or in a combination thereof. An antibody can include at least 1, 2, 3, 4, 5, 6, 7, 8 or more cysteines having a structure of Formula II or Formula III. Alternatively or additionally, an antibody can include at most 8, 7, 6, 5, 4, 3, 2, or 1 cysteines having a structure of Formula II or Formula III. The cysteines having a structure of Formula II or Formula III can be native cysteines (e.g. cysteines that are conserved in the antibody structure). Alternatively, one or both of the cysteines can be introduced via genetic engineering or synthetic techniques.

Formula II or Formula III can form a linear, non-cyclic structure. For example, the alkyl, alkenyl, alkynyl, aryl, heteroalkyl, heteroalkenyl, heteroalkynyl, heteroaryl or substituted variant of Formula II or Formula III can form a linear structure. Alternatively, Formula II or Formula III can form a cyclic structure. For example, two or more of R¹, R², R³, R⁴ or R⁵ of Formula II can occupy a ring structure. Similarly, two or more of R¹, R², or R³ of Formula III can occupy a ring structure.

An antibody conjugate of the present disclosure can include an attachment moiety connected to the antibody conjugate via a moiety of Formula II or Formula III. For example, one or more of R¹, R², R³, R⁴ or R⁵ of Formula II can be connected to an attachment moiety. Similarly, one or more of R¹, R², or R³ of Formula III can be connected to an attachment moiety. Any of a variety of attachment moieties can be used including, but not limited to those set forth above in the context of antibody conjugates having cysteine moieties of Formula I.

An antibody conjugate of the present disclosure can include a label moiety connected to the antibody conjugate via a moiety of Formula II or Formula III. For example, one or more of R¹, R², R³, R⁴ or R⁵ of Formula II can be connected to a label moiety. Similarly, one or more of R¹, R², or R³ of Formula III can be connected to a label moiety. Any of a variety of label moieties can be used including, but not limited to those set forth above in the context of antibody conjugates having cysteine moieties of Formula I.

An antibody conjugate of the present disclosure can be connected to a particle via a moiety of Formula II or Formula III. For example, one or more of R¹, R², R³, R⁴ or R⁵ of Formula II can be connected to a particle. Similarly, one or more of R¹, R², or R³ of Formula III can be connected to a particle. Any of a variety of particles can be used including, but not limited to those set forth above in the context of antibody conjugates having cysteine moieties of Formula I.

An antibody conjugate of the present disclosure can be connected to a solid support via a moiety of Formula II or Formula III. For example, one or more of R¹, R², R³, R⁴ or R⁵ of Formula II can be connected to a solid support. Similarly, one or more of R¹, R², or R³ of Formula III can be connected to a solid support. Any of a variety of solid supports can be used including, but not limited to those set forth above in the context of antibody conjugates having cysteine moieties of Formula I. In some cases, the solid support has an array of addresses that are competent for attachment of antibody conjugates, or attachment of a plurality of antibody conjugate forms an array of antibody conjugates.

The variants of Formula II or Formula III exemplified herein in the context of antibodies, such as those set forth above, can be present in proteins or other analytes.

The present disclosure provides an array including a plurality of addresses and a plurality of antibodies, wherein individual addresses of the plurality of addresses are each attached to an antibody of the plurality of antibodies, wherein each of the individual addresses is attached to a modified cysteine of the antibody, the modified cysteine comprising a moiety of Formula II:

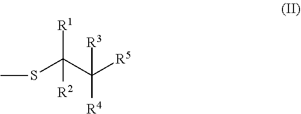

### or Formula III:

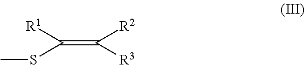

wherein S is the sulfur moiety of the cysteine, and wherein R¹, R², R³, R⁴ and R⁵ are each independently selected from any of a variety of atoms or organic moieties, and wherein at least one of R¹, R², R³, R⁴ and R⁵ includes a linker connecting an antibody of the plurality of antibodies to an individual addresses of the plurality of addresses. Optionally, R¹, R², R³, R⁴ and R⁵ are each independently selected from the group consisting of hydrogen, halo, alkyl, alkenyl, alkynyl, aryl, heteroaryl, heteroalkyl, and optionally substituted variants thereof. The array can be configured as exemplified above in the context of antibody conjugates having a structure of Formula I.

An antibody conjugate can include a linker connecting a cysteine of Formula II or Formula III to an object, substance or moiety. In particular configurations, a cysteine of Formula II or Formula III is connected to an object, substance or moiety via the linker. Useful linker moieties include, but are not limited to, those set forth above in the context of antibody conjugates having a structure of Formula I.

Also provided herein is a method of modifying an antibody, including reacting a cysteine of an antibody, with an alkene or alkyne moiety of a modifying reagent, wherein the alkene moiety reacts with the cysteine to form a moiety of Formula II:

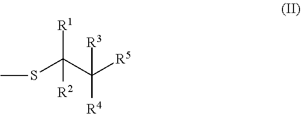

wherein the alkyne moiety reacts with the cysteine to form a moiety of Formula III:

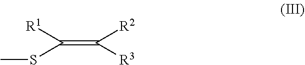

wherein S represents the sulfur moiety of the cysteine, and wherein R¹, R², R³, R⁴ and R⁵ are each independently selected from any of a variety of atoms or organic moieties. Optionally, R¹, R², R³, R⁴ and R⁵ are each independently selected from the group consisting of hydrogen, halo, alkyl, alkenyl, alkynyl, aryl, heteroalkyl, heteroalkenyl, heteroalkynyl, heteroaryl, and optionally substituted variants thereof.

A method for modifying an antibody with an alkene or alkyne to produce an antibody conjugate can be carried out under irradiation with light to drive formation of one or more cysteines having a moiety of Formula II or Formula III. Without intending to be limited by mechanism, the reaction can be considered, at least in some configurations of the methods set forth herein, to be a photo thiol-ene reaction or a photo thiol-yne reaction. Light can be provided at a wavelength or wavelength range that is compatible with the reaction components. For example, the light can be in a range of the spectrum exemplified above in the context of making an antibody conjugate having a moiety of Formula I. Moreover, the light can be delivered for a period of time and at a radiant flux exemplified in the context of making an antibody conjugate having a moiety of Formula I.

The use of light to drive formation of an antibody conjugate having a moiety of Formula II or Formula III can be particularly useful when the alkene or alkyne moiety of the modifying reagent is not present in a strained ring. For example, the alkene moiety or alkyne moiety can be in a linear hydrocarbon or in a ring having at least 6, 7, 8 or more members. However, a method for making an antibody conjugate can include delivery of light to a reaction that occurs between a cysteine and an alkene or alkyne moiety that is present in a strained ring. A method of modifying an antibody to produce an antibody conjugate having a moiety of Formula II or Formula III can employ a modifying reagent having a cyclooctene, cycloheptene, cyclohexene, cyclopentene, cyclooctyne, cycloheptyne, cyclohexyne or cyclopentyne moiety. A dibenzocyclooctyne moiety can be particularly useful. Other cyclooctyne moieties can be particularly useful such as those derived from 3,3-difluoro-substituted cyclo-1-octyne and cyclopropane-fused bicyclo[6.1.0]nonyne. Optionally, the reactions can be carried out in a spatially resolved way by spatially resolved delivery of light. For example, light can be delivered to at least a first subregion of a tissue, analyte array or other sample having a surface area, whereas the light does not contact at least one other subregion of the sample. Reagents can be selected such that subregions of the sample that are not irradiated with light will not produce a photoproduct even if the reagents are in contact with those subregions. However, subregions that are in contact with the reagents and also irradiated by light will produce a photoproduct. The photoreaction can be used to add a label, tag (e.g. nucleic acid tag), or other detectable moiety for subsequent assay to identify or characterize components of the sample.

In some configurations of the methods for making an antibody conjugate having a moiety of Formula II or Formula III, light is not delivered to a reaction in which an alkene or alkyne moiety is reacted with a cysteine. For example, the reaction can be carried out in absence of light or under standard laboratory lighting that is not enriched for light having wavelengths at or below the UV range or other spectral range set forth herein. Reactions that are not activated by light can be carried out using an alkene or alkyne that is activated by other mechanisms, such as via ring strain.

A method of the present disclosure can be carried out to produce an antibody conjugate having at least 1, 2, 3, 4, 5, 6, 7, 8 or more cysteines having a moiety of Formula II or Formula III. Alternatively or additionally, a method of the present disclosure can be carried out to produce an antibody conjugate having at most 8, 7, 6, 5, 4, 3, 2 or 1 cysteines having a moiety of Formula II or Formula III. The number of cysteine pairs that are modified can be controlled, for example, via genetic engineering to add, remove or relocate cysteines in the antibody structure, via selective reduction of some cysteines compared to others in the antibody prior to reaction with a modifying reagent having an alkene or alkyne moiety, via selective oxidation of some cysteines compared to others in the antibody prior to reaction with a modifying reagent having an alkene or alkyne moiety, or via selective reaction of reduced cysteines with an alkene or alkyne moiety, or a combination thereof.

A method of modifying an antibody to produce an antibody conjugate having a moiety of Formula II or Formula III can be configured to react an alkene or alkyne moiety with a sulfur moiety on a heavy chain of the antibody, light chain of the antibody or both. The method can produce an antibody conjugate having a moiety of Formula II or Formula III on at least 1, 2, 3 or more cysteine(s) on a heavy chain of the antibody conjugate. Alternatively or additionally, the method can produce an antibody conjugate having a moiety of Formula II or Formula III on at most 3, 2, or 1 cysteine(s) on a heavy chain of the antibody conjugate. Independent of the presence or number of modified cysteines on a first heavy chain, a method set forth herein can produce an antibody conjugate having a moiety of Formula II or Formula III on at least 1, 2, 3 or more cysteine(s) on a second heavy chain of the antibody conjugate. Alternatively or additionally, the method can produce an antibody conjugate having a moiety of Formula II or Formula III on at most 3, 2, or 1 cysteine(s) on a second heavy chain of the antibody conjugate. Moreover, independent of the presence or number of modified cysteines on one or both heavy chains, an antibody conjugate can have a moiety of Formula II or Formula III on a cysteine in one or both light chains.

An antibody that is to be modified to have a moiety of Formula II or Formula III, may contain pairs of cysteines that are bridged by a disulfide. These oxidized cysteines can be reduced prior to being reacted with an alkene or alkyne moiety to produce an antibody conjugate. All of the cysteines can be reduced or a subset of the cysteines can be selectively reduced. Cysteines can be reduced using reagents and methods set forth above in the context of producing antibody conjugates having paired cysteines with a two-carbon bridge. Moreover, an antibody that is modified to include a moiety of Formula II or Formula III can be genetically engineered to remove or add cysteines, for example, as set forth above in the context of making antibody conjugates having paired cysteines with a two-carbon bridge.

A modifying reagent that is used in a method set forth herein to produce an antibody conjugate having a moiety of Formula II or Formula III can be connected to an attachment moiety, label moiety, linker moiety, solid support or particle (e.g. a structured nucleic acid particle). These moieties can be configured and used as set forth above in the context of making antibody conjugates having paired cysteines with a two-carbon bridge.

The methods and compositions set forth herein, although exemplified on the context of modifying antibodies, can be extended to modification of other biochemical entities. For example, the methods set forth herein for reacting an alkyne moiety with a pair of cysteines of an antibody to form a two-carbon bridge between the sulfur atoms of the cysteine can be used to form a two-carbon bridge between a pair of cysteines in a protein or between a cysteine on a protein and a sulfur moiety on another substance or object. Any of a variety of substances or objects can be used including, for example, a second protein having a sulfhydryl such as a cysteine sulfhydryl, a chemically modified nucleic acid having a sulfhydryl, a structured nucleic acid particle (or other particle) having a sulfhydryl, a solid support having a sulfhydryl such as a glass surface modified with thiosilanes, or the like.

Accordingly, the present method provides a method that includes reacting an alkyne moiety of a modifying reagent with a sulfur moiety of a protein and a sulfur moiety of a substance or object to form a moiety of Formula I:

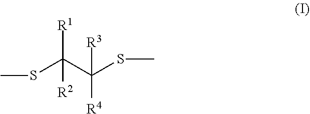

wherein one of the S moieties is the sulfur moiety of the protein and the other S moiety is the sulfur moiety of the substance or object, and wherein R¹, R², R³, and R⁴ are each independently selected from the group consisting of hydrogen, halo, alkyl, alkenyl, alkynyl, aryl, heteroalkyl, heteroalkenyl, heteroalkynyl, heteroaryl, and optionally substituted variants thereof.

Also provided herein is a composition including a protein covalently attached to a substance or object, wherein a two-carbon bridge covalently connects a sulfur atom of the protein to a sulfur moiety of the substance or object. For example, the present disclosure provides a composition, including a moiety of Formula I:

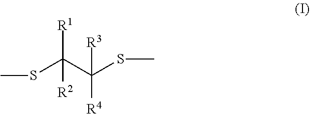

wherein one of the S moieties is the sulfur moiety of the protein and the other S moiety is the sulfur moiety of the substance or object, and wherein R¹, R², R³, and R⁴ are each independently selected from the group consisting of hydrogen, halo, alkyl, alkenyl, alkynyl, aryl, heteroalkyl, heteroalkenyl, heteroalkynyl, heteroaryl, and optionally substituted variants thereof.

Optionally, the substance or object included in the above method or composition can be one or more of those exemplified herein in the context of modified antibodies, or one or more of those known in the art.

In some cases, an object that is crosslinked to a protein in a composition set forth herein or by a method set forth herein can be an address of an array. The crosslink can be a two-carbon bridge covalently connecting a sulfur atom of the protein to a sulfur moiety of the address. Accordingly, the present disclosure provides an array including a plurality of addresses and a plurality of proteins, wherein each of the proteins includes a sulfur moiety crosslinked via a two-carbon bridge covalently connecting the sulfur moiety to an address of the plurality of addresses. For example, the array can include a plurality of addresses and a plurality of proteins, wherein individual addresses of the plurality of addresses are each attached to a protein of the plurality of proteins via a moiety of Formula I:

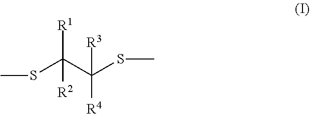

wherein one of the S moieties is the sulfur moiety of the protein and the other S moiety is the sulfur moiety of an address of the array, and wherein R¹, R², R³, and R⁴ are each independently selected from the group consisting of hydrogen, halo, alkyl, alkenyl, alkynyl, aryl, heteroalkyl, heteroalkenyl, heteroalkynyl, heteroaryl, and optionally substituted variants thereof.

Methods or composition that are exemplified herein in the context of antibodies can be applied to other proteins having pairs of sulfurs, such as pairs of cysteine sulfhydryls. Accordingly, the present disclosure provides a protein, including a pair of sulfurs, such as cysteine sulfurs, crosslinked via a two-carbon bridge covalently connecting the sulfurs. For example, the present disclosure provides a protein, including a pair of sulfurs crosslinked via a moiety of Formula I:

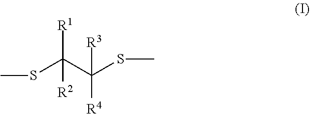

wherein S represents the sulfurs of the pair, and wherein R¹, R², R³, and R⁴ are each independently selected from any of a variety of atoms or organic moieties. In particular configurations, R¹, R², R³, and R⁴ are each independently selected from the group consisting of hydrogen, halo, alkyl, alkenyl, alkynyl, aryl, heteroalkyl, heteroalkenyl, heteroalkynyl, heteroaryl, and optionally substituted variants thereof.

The present disclosure further provides an array including a plurality of addresses and a plurality of proteins, wherein each of the proteins includes a pair of sulfurs, such as cysteine sulfurs, crosslinked via a two-carbon bridge covalently connecting the sulfur atoms, and wherein the two-carbon bridge further comprises a linker attached to an address of the plurality of addresses. For example, the array can include a plurality of addresses and a plurality of proteins, wherein individual addresses of the plurality of addresses are each attached to a protein of the plurality of proteins, wherein each of the individual addresses is attached to a pair of modified sulfurs of the protein, the modified sulfurs having a moiety of Formula I:

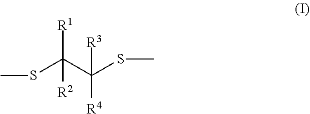

wherein S represents the sulfur moieties of the pair, wherein R¹, R², R³, and R⁴ are each independently selected from any of a variety of atoms or organic moieties, and wherein at least one of R¹, R², R³, and R⁴ comprises a linker connecting a protein of the plurality of proteins to an individual address of the plurality of addresses. Optionally, R¹, R², R³, and R⁴ are each independently selected from the group consisting of hydrogen, halo, alkyl, alkenyl, alkynyl, aryl, heteroalkyl, heteroalkenyl, heteroalkynyl, heteroaryl, and optionally substituted variants thereof.

The present disclosure provides a method of modifying a protein by reacting an alkyne moiety of a modifying reagent with a pair of sulfurs, such as cysteine sulfurs, thereby forming a two-carbon bridge between the sulfurs of the pair. For example, the method can include reacting a pair of sulfurs of a protein, with an alkyne moiety of a modifying reagent, wherein the alkyne moiety reacts with the pair of sulfurs to form a moiety of Formula I:

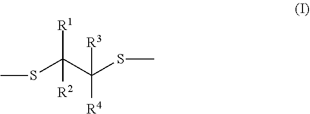

wherein S represents the sulfur moieties of the pair, and wherein R¹, R², R³, and R⁴ are each independently selected from any of a variety of atoms or organic moieties. Optionally, R¹, R², R³, and R⁴ are each independently selected from the group consisting of hydrogen, halo, alkyl, alkenyl, alkynyl, aryl, heteroalkyl, heteroalkenyl, heteroalkynyl, heteroaryl, and optionally substituted variants thereof.

In the above methods or compositions, a sulfur moiety of the protein can be provided by a cysteine or chemical modification. The cysteine can be native to the protein or a non-native amino acid, such as a product of a protein engineering process.

Formula I, when present in a protein other than an antibody, can include a structure or function set forth herein in the context of modified antibodies. For example, one or more of R¹, R², R³, and R⁴ can include a linker that is attached to a second substance or second object. As such, Formula I can optionally provide a ternary crosslink between (i) the protein, (ii) the substance or object, and (iii) the second substance or object. The second substance or object can be one or more of those exemplified herein in the context of modified antibodies, or one or more of those known in the art. The substance and second substance can be the same type of substance, for example, both can be nucleic acids (having the same or different nucleotide sequences) or proteins (having the same or different amino acid sequences). Alternatively, the second substance can be different from the substance. Similarly, the object and second object can be the same type of object, for example, both can be structured nucleic acid particles (having the same or different nucleotide sequences) or solid supports (having the same or different composition). Alternatively, the second object can be different from the object. Any of a variety of linkers can be used including, but not limited to those exemplified herein in the context of modified antibodies.

A photo thiol-ene or photo thiol-yne reaction can be used to attach a protein to an object such as a particle or solid support (e.g. at an address in an array on a solid support). The attachment can occur via photoaddition at a cysteine sulfur of the protein. The photo thiol-ene and photo thiol-yne reactions provide a relatively specific protein modification at cysteines in a single step. This is advantageous compared to modification of other amino acid types that are either less specific or require multiple steps to achieve specificity, such as chemistries that target amines or carbonyls. Of course, it will be understood that a protein can be pre-modified to introduce a sulfur moiety and the sulfur moiety can then be modified using a photo thiol-ene or photo thiol-yne reaction set forth herein. For example, amines such as those resent at the N-terminus of a protein or at lysine sidechains can be modified using Traut's reagent (2-iminothiolane) or using SATA (N-succinimidyl S-acetylthioacetate) to produce sulfhydryl-modified amines. Carboxylates, such as those present at the carboxy terminus or at aspartate or glutamate sidechains can also be functionalized with sulfhydrils for example using a compound that contains both an amine and a disulfide. The disulfide effectively protects the thiol and then 1-ethyl-3-(3-dimethylaminopropyl)carbodiimide (EDC)-based coupling can occur between the carboxylic acid side chain and the amine.

In a particular configuration, a method or composition set forth herein can attach a protein to a structured nucleic acid particle. A structured nucleic acid particle can be engineered to include a single alkyne moiety (or a single alkene moiety) that is photoreactive with sulfur, thereby facilitating attachment of a single protein to the particle. The resulting particle-protein conjugate can be useful for analytical or preparative methods that are performed as single-analyte resolution. Moreover, a plurality of structured nucleic particles, each having a single reactive alkene or alkyne moiety, can be subjected to photo thiol-yne reaction (or photo thiol-ene reaction) with a plurality of different proteins to form a plurality of protein-particle conjugates, wherein each of the conjugates includes a single protein attached per particle. The particles can facilitate manipulation or detection of the attached proteins at single-analyte resolution. For example, the protein-particle conjugates can be spatially separated on a solid support or temporally separated in a fluid stream to provide an array of individually resolvable proteins.

Methods for covalently attaching a protein to a particle or solid support often include pre-modification of the protein with a functional moiety which offers a specific reactivity for such attachment. This is often achieved by protein modifications with certain bio-orthogonal moieties—those that are not reactive with amino acid residues until triggered—such as methyl tetrazine (mTz) or azide (N₃) among others. For example, many techniques involve covalent attachment of these moieties at lysine residues through amide bond formation. This amide attachment offers convenience given current access to various types of amine-reactive reagents for these groups and their applicable protocols. However, this amide method can result in undesirably high product distributions and corresponding heterogeneities due to the relatively high prevalence of lysines in proteins. Furthermore, reactivity of lysines to these reagents is influenced by the number and type of amino acids in proximity to the lysines. Also, the range in the number of lysines per protein is relatively high in typical proteomes, for example, compared to the range of other amino acid types, such as cysteines in the proteomes. This can lead to complications when processing a sample having a population of different proteins such that the proteins differ with respect to the number of lysines present and the accessibility of lysines to modifying reagents. These issues can be substantial for protein mixtures from biological samples such as cell lysates or other proteome-scale samples.

Cysteines, being less prevalent than lysines and present in a tighter range of prevalence per protein, provide a useful target for modification to avoid or at least minimize complications arising when targeting lysines. Cysteine modification chemistries are available including, for example, those that employ thiol-reactive bifunctional reagents such as those containing a moiety of Michael acceptor (maleimide, acrylate, acrylamide, or their congeners) or iodoacetamide. However, despite their cysteine selectivity, their utility and modification efficiency are compromised given reported problems of linkage instability (e.g., cleavage of thiol-maleimide linkage in aqueous buffer solutions) or relatively low yields of such cysteine modifications.

The use of photo thiol-ene and photo thiol-yne as set forth herein can address unmet needs in protein modification. Unlike other cysteine modification methods, such as thermal thiol-maleimide conjugation, methods set forth herein can facilitate formation of a thiol-alkene or thiol-alkyne linkage under photo catalytic conditions. Such reaction conditions are robust and well tolerated for modifying a single target protein as well as a diverse protein mixture such as cell lysate proteins. Compared to conventional amide or cysteine methods illustrated above, this method offers several beneficial features that are comparable or better. These include site-selective protein modifications at cysteine, greater homogeneity in product distribution, and a shorter reaction time (e.g. mere minutes compared to several hours or a full day needed in other methods). Unlike the thiol-maleimide linkage, which is labile, thiol-alkene and thiol-alkyne linkages remain stable in physiological buffer solutions.

Optionally, a photo thiol-ene and photo thiol-yne reaction can be carried out on a surface of a sample, such as a tissue, analyte array or collection of cells in a fluid stream. Different subregions on the sample can be differentially exposed to the light. For example, at least a first subregion can be irradiated under conditions for photo thiol-ene and photo thiol-yne reaction and at least a second subregion can be non-irradiated. The first subregion can include a sulfur-bearing target analyte (e.g. a protein) and a reagent bearing an alkene or alkyne moiety, thereby producing a photoproduct. Absence of irradiation for the second subregion can inhibit the reaction from occurring, even if the second subregion includes both a sulfur-bearing target and a reagent bearing an alkene or alkyne moiety.

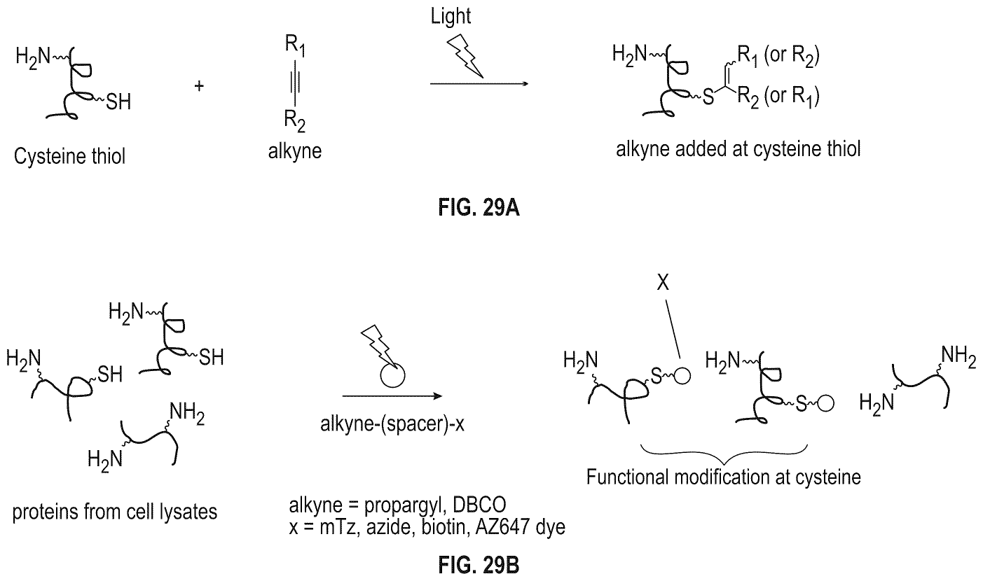

FIG. 29A shows a photo crosslinking reaction that occurs between an alkyne molecule and a thiol moiety at cysteine under a photo thiol-yne condition. In particular configurations, R¹ and R² are each independently selected from the group consisting of hydrogen, halo, alkyl, alkenyl, alkynyl, aryl, heteroalkyl, heteroalkenyl, heteroalkynyl, heteroaryl, and optionally substituted variants thereof. Optionally, R¹ or R² (or both) comprise a linkage to a solid support, particle or other object.

FIG. 29B shows a general scheme that depicts functional modifications of proteins at cysteines by photo reaction with alkyne-spacer-x, a dual-functional molecule, under the photo thiol-yne condition. Optionally the alkyne can be a reagent set forth herein such as propargyl or DBCO, or other reagent known in the art. Reactant x can be mTz, azide, biotin, AZ647 dye, or other reagent known in the art or set forth herein.

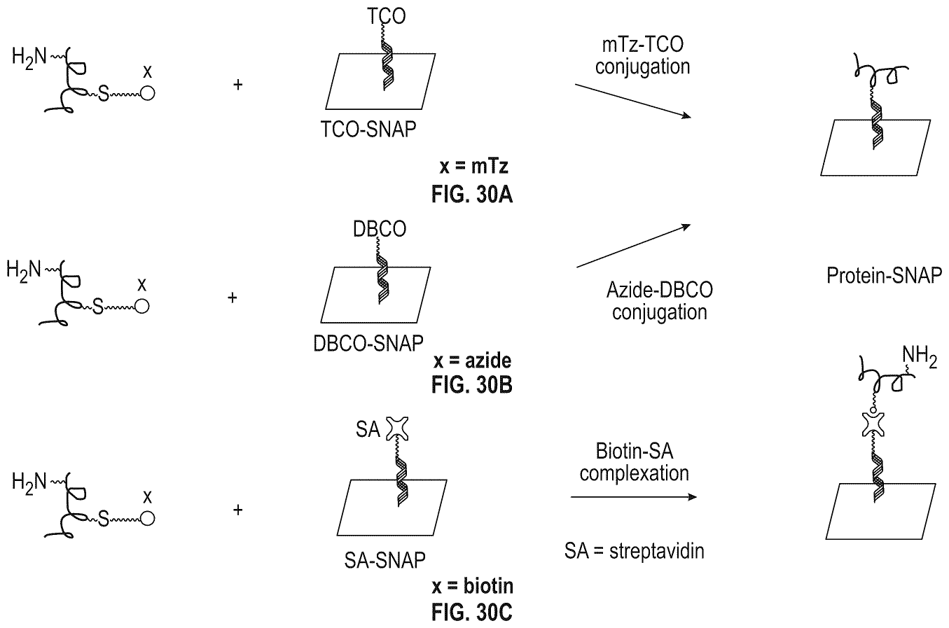

Photoconjugation methods, such as photo thiol-ene or photo thiol-yne reactions can be used to attach proteins to a variety of objects, such as structured nucleic acid particles. FIG. 30A shows a reaction for covalent attachment of an mTz-modified protein at a TCO-presenting structured nucleic acid particle (SNAP) through inverse electron demand Diels-Alder (IEDDA) reaction. FIG. 30B shows a reaction for covalent attachment of an azide-modified protein at a DBCO-presenting SNAP through strain-promoted azide-alkyne click (SPAAC) reaction. FIG. 30C shows a reaction for non-covalent complexation of a biotin-modified protein with streptavidin (SA) presented on a SNAP. The structured nucleic acid particles can have nucleic acid origami structures or other compositions set forth herein for such particles.

Methods set forth herein for attaching proteins to objects, such as particles or solid supports, can utilize any of a variety of protein samples. Proteomes from biological samples can be used. The number and variety of proteins captured from a given proteome can at least in part be a function of the fraction of proteins in the proteome that have at least one cysteine. For example, 97% of the proteins in the H. sapiens proteome are estimated to have at least one cysteine, 94% of the proteins in the A. thaliana proteome are estimated to have at least one cysteine, 94% of the proteins in the D. melanogaster proteome are estimated to have at least one cysteine, 92% of the proteins in the O. sativa proteome are estimated to have at least one cysteine, 90% of the proteins in the S. cerevisiae proteome are estimated to have at least one cysteine, 84% of the proteins in the E. coli proteome are estimated to have at least one cysteine, and 60% of the proteins in the T. gammatolerans proteome are estimated to have at least one cysteine. See Weidemann et al. Frontiers in Chem. vol 8, article 280 (2020), which is incorporated herein by reference. As such, a method set forth herein can be configured to modify at most the above faction of the proteins from the respective proteome. While staying within these upper limits, an attachment reaction set forth herein can be configured to modify at least 10%, 25%, 50%, or 75% of the proteins from the respective proteome.

A plurality of proteins, whether attached to particles, present in an array or in any of a variety of other compositions set forth herein, can be characterized in terms of total protein mass. A plurality of proteins can include at least 1 pg, 10 pg, 100 pg, 1 ng, 10 ng, 100 ng, 1 ug, 10 ug, 100 ug, 1 mg, 10 mg, 100 mg or more protein by mass. Alternatively or additionally, a plurality of proteins may contain at most 100 mg, 10 mg, 1 mg, 100 ug, 10 ug, 1 ug, 100 ng, 10 ng, 1 ng, 100 pg, 10 pg, 1 pg or less protein by mass.

A plurality of proteins can be characterized in terms of total number of protein molecules. A plurality of proteins used or included in a method, composition or apparatus set forth herein can include at least 2 protein molecules, 10 protein molecules, 100 protein molecules, 1×10⁴ protein molecules, 1×10⁶ protein molecules, 1×10⁸ protein molecules, 1×10¹⁰ protein molecules, 1 mole (6.02214076×10²³ molecules) of protein molecules, 10 moles of protein molecules, 100 moles of protein molecules or more. Alternatively or additionally, a plurality of proteins may contain at most 100 moles of protein molecules, 10 moles of protein molecules, 1 mole of protein molecules, 1×10¹⁰ protein molecules, 1×10⁸ protein molecules, 1×10⁶ protein molecules, 1×10⁴ protein molecules, 100 protein molecules, 10 protein molecules, or less.

A plurality of proteins can be characterized in terms of the variety of full-length amino acid sequences in the plurality. For example, the variety of full-length amino acid sequences in a plurality of proteins can be equated with the number of different protein-encoding genes in the source for the plurality of proteins. Whether or not the proteins are derived from a known genome or from any genome at all, the variety of full-length amino acid sequences can be counted independent of presence or absence of post translational modifications in the proteins. A plurality of proteins used or included in a method, composition or apparatus set forth herein can have a complexity that includes substantially all different native-length amino acid sequences from a given source. A proteome or subfraction can have a complexity of at least 2, 5, 10, 100, 1×10³, 1×10⁴, 2×10⁴, 3×10⁴ or more different native-length amino acid sequences. Alternatively or additionally, a proteome or subfraction can have a complexity that is at most 3×10⁴, 2×10⁴, 1×10⁴, 1×10³, 100, 10, 5, 2 or fewer different native-length amino acid sequences.

The diversity of a proteomic sample can include at least one representative for substantially all proteins encoded by a source from which the sample was derived or a substantial fraction thereof. For example, a proteomic sample may contain at least one representative for at least 60%, 75%, 90%, 95%, 99%, 99.9% or more of the proteins encoded by a source from which the sample was derived. Alternatively or additionally, a proteomic sample may contain a representative for at most 99.9%, 99%, 95%, 90%, 75%, 60% or less of the proteins encoded by a source from which the sample was derived.

A plurality of proteins can be characterized in terms of the variety of full-length amino acid sequences in the plurality including transcribed splice variants. The human proteome has been estimated to include about 70,000 different full-length amino acid sequences when splice variants ae included. See Aebersold et al., Nat. Chem. Biol. 14:206-214 (2018), which is incorporated herein by reference. A plurality of proteins used or included in a method, composition or apparatus set forth herein can have a complexity of at least 2, 5, 10, 100, 1×10³, 1×10⁴, 7×10⁴, 1×10⁵, 1×10⁶ or more different full-length amino acid sequences. Alternatively or additionally, a plurality of proteins can have a complexity that is at most 1×10⁶, 1×10⁵, 7×10⁴, 1×10⁴, 1×10³, 100, 10, 5, 2 or fewer different full-length amino acid sequences.

A plurality of proteins can be characterized in terms of the variety of protein structures therein including, for example, different full-length amino acid sequences or different proteoforms among those sequences. Different molecular forms of proteins expressed from a given gene are considered to be different proteoforms. Proteoforms can differ, for example, due to differences in primary structure (e.g. shorter or longer amino acid sequences), different arrangement of domains (e.g. transcriptional splice variants), or different post translational modifications (e.g. presence or absence of phosphoryl, glycosyl, acetyl, or ubiquitin moieties). The human proteome is estimated to include hundreds of thousands of proteins when counting the different primary structures and proteoforms. See Aebersold et al., Nat. Chem. Biol. 14:206-214 (2018), which is incorporated herein by reference. A plurality of proteins used or included in a method, composition or apparatus set forth herein can have a complexity of at least 2, 5, 10, 100, 1×10³, 1×10⁴, 1×10⁵, 1×10⁶, 5×10⁶, 1×10⁷ or more different protein structures. Alternatively or additionally, a plurality of proteins can have a complexity that is at most 1×10⁷, 5×10⁶, 1×10⁶, 1×10⁵, 1×10⁴, 1×10³, 100, 10, 5, 2 or fewer different protein structures.

A plurality of proteins can be characterized in terms of the dynamic range for the different protein structures in the plurality. The dynamic range can be a measure of the range of abundance for all different protein structures in a plurality of proteins, the range of abundance for all different primary protein structures in a plurality of proteins, the range of abundance for all different full-length primary protein structures in a plurality of proteins, the range of abundance for all different full-length gene products in a plurality of proteins, the range of abundance for all different proteoforms expressed from a given gene, or the range of abundance for any other set of different proteins set forth herein. The dynamic range for plurality of proteins set forth herein can be a factor of at least 10, 100, 1×10³, 1×10⁴, 1×10⁶, 1×10⁸, 1×10¹⁰, or more. Alternatively or additionally, the dynamic range for plurality of proteins set forth herein can be a factor of at most 1×10¹⁰, 1×10⁸, 1×10⁶, 1×10⁴, 1×10³, 100, 10 or less.

A protein or other analyte can be derived from a natural or synthetic source. Exemplary sources include, but are not limited to a biological tissue, fluid, cell or subcellular compartment (e.g. organelle). For example, a sample can be derived from a tissue biopsy, biological fluid (e.g. blood, plasma, extracellular fluid, urine, mucus, saliva, semen, vaginal fluid, sweat, synovial fluid, lymph, cerebrospinal fluid, peritoneal fluid, pleural fluid, amniotic fluid, intracellular fluid, extracellular fluid, etc.), fecal sample, hair sample, cultured cell, culture media, fixed tissue sample (e.g. fresh frozen or formalin-fixed paraffin-embedded) or protein synthesis reaction. A primary source for a cancer biomarker protein may be a tumor biopsy sample. Other sources for proteins and analytes include environmental samples or forensic samples.

Exemplary organisms from which a protein or other analyte can be derived include, but are not limited to, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a dictyostelium discoideum; a fungi such as Pneumocystis carinii, Takifugu rubripes, yeast, Saccharamoyces cerevisiae or Schizosaccharomyces pombe; or a Plasmodium falciparum. A protein can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid. A protein or other analyte can be derived from a homogeneous culture or population of the above organisms or alternatively from a collection of several different organisms, for example, in a community or ecosystem.

In some cases, a protein or other analyte can be derived from an organism that is collected from a host organism. A protein or other analyte may be derived from a parasitic, pathogenic, symbiotic, or latent organism collected from a host organism. A protein or other analyte can be derived from an organism, tissue, cell or biological fluid that is known or suspected of being associated with a disease state or disorder (e.g., an oncogenic virus). Alternatively, a protein or other analyte can be derived from an organism, tissue, cell or biological fluid that is known or suspected of not being associated with a particular disease state or disorder. For example, one or more proteins isolated from such a source can be used as a control for comparison to results acquired from a source that is known or suspected of being associated with the disease state or disorder. A sample may include a microbiome. A sample may include a plurality of proteins or other analytes of interest contributed by microbiome constituents. In some cases, one or more proteins (or other analytes) used in a method, composition or apparatus set forth herein may be obtained from a single organism (e.g. an individual human), single cell, single organelle, or single protein-containing particle (e.g., a viral particle).

One or more analytes can optionally be separated or isolated from other components of the source for the analyte(s). For example, one or more proteins can be separated or isolated from other biological components such as lipids, nucleic acids, hormones, enzyme cofactors, vitamins, metabolites, microtubules, organelles (e.g. nucleus, mitochondria, chloroplast, endoplasmic reticulum, vesicle, cytoskeleton, vacuole, lysosome, cell membrane, cytosol or Golgi apparatus), other proteins or the like. Protein separation can be carried out using methods known in the art such as centrifugation (e.g. to separate membrane fractions from soluble fractions), density gradient centrifugation (e.g. to separate different types of organelles), precipitation, affinity capture, adsorption, liquid-liquid extraction, solid-phase extraction, chromatography (e.g. affinity chromatography, ion exchange chromatography, reverse phase chromatography, size exclusion chromatography, electrophoresis (e.g. polyacrylamide gel electrophoresis) or the like. Useful protein separation methods are set forth in Scopes, Protein Purification Principles and Practice, Springer; 3rd edition (1993).

A protein that is used in a composition or method set forth herein can be in a native or denatured conformation. For example, a protein can be in a native conformation, whereby it is capable of performing native function(s) such as catalysis of its natural substrate(s) or binding to its natural substrate(s). Alternatively, a protein can be in a denatured conformation whereby it is incapable of performing certain native function(s) such as catalysis of its natural substrate(s) or binding to its natural substrate(s). A protein can be in a native conformation for some manipulations set forth herein and in a denatured conformation for other manipulations set forth herein. For example, a protein can be in a native state when subjected to a photoreaction and then denatured for one or more subsequent manipulations such as deposition on an array or detection. Alternatively, a protein can be in a denatured state when subjected to a photoreaction set forth herein. A protein may be denatured at any stage during manipulation, including for example, upon removal from a native milieu or at a later stage of processing such as a stage where the protein is separated from other cellular components, fractionated from other proteins, functionalized to include a reactive moiety, attached to a particle or solid support, contacted with an affinity reagent, detected, or other manipulation. Any of a variety of denaturants can be used such as heat (e.g. temperatures greater than about 40° C., 60° C., 80° C. or higher), excessive pH (e.g. pH lower than 4.0, 3.0 or 2.0; or pH greater than 10.0, 11.0 or 12.0); chaotropic agents (e.g. urea, guanidinium chloride, or sodium dodecyl sulfate), organic solvent (e.g. chloroform or ethanol), physical agitation (e.g. sonication) or radiation. A denatured protein may be refolded, for example, reverting to a native state for one or more steps of a process set forth herein.

A sample used herein, whether containing proteins or other analytes, need not be from a biological source and can instead be from an artificial source, such as a library from a combinatorial synthesis or a library from an in vitro synthesis that exploits biological components. An artificial sample can have a plurality of proteins or other analytes of interest that include mass, number of molecules, diversity, complexity dynamic range or other quantifiable characteristic similar to those set forth herein for proteomes.

### Example I

### Modification of IgG1 with Dibenzocyclooctyne-AZDye™ 647 Modifying Reagent

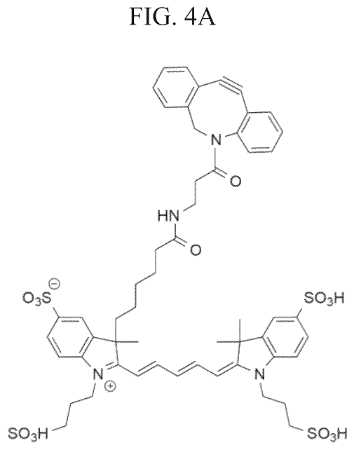

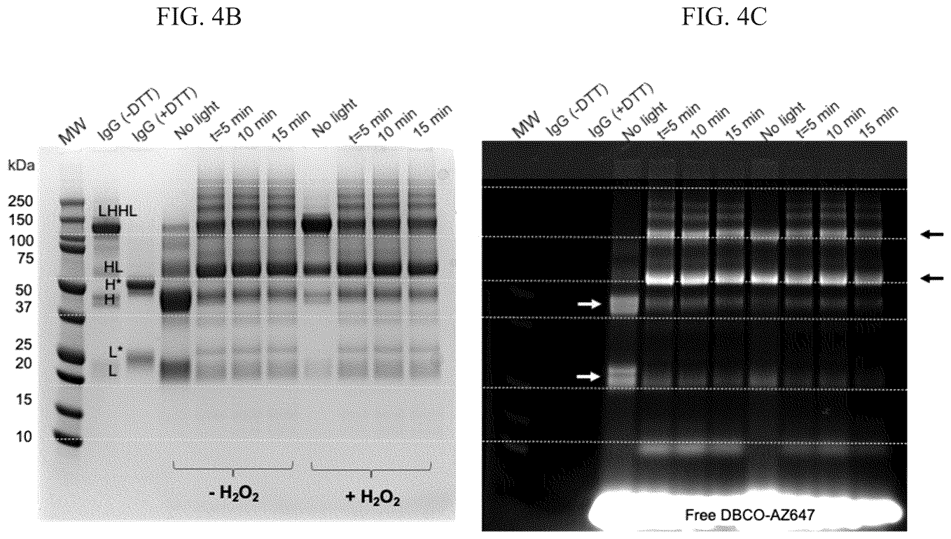

IgG1 antibody (unlabeled rabbit IgG₁, Southern Biotech) dissolved in borate buffered saline (BBS) pH 8.2 (1 mg/mL) was reacted with tenfold molar excess of TCEP at 5° C. for twelve hours to selectively reduce interchain disulfide bonds located at heavy (H)-to-light (L) chains and a heavy (H) chain-to-heavy (H) chain . The generated sulfhydryls of the reduced IgG1 antibody were then reacted with dibenzocyclooctyne-AZDye™ 647 (DBCO-AZ647; from Click Chemistry Tools, Cat# 1302; see FIG. 4A) in the presence of 8-fold molar equivalent of azobis(2-methylpropionamidine) dihydrochloride (AAPH; Cat#440914; Sigma-Aldrich) under irradiation with light at 365 nm for variable times between 5 and 15 minutes to produce an antibody conjugate modified to include the AZDye™ 647 (lex=648 nm, lem=671 nm). Following the photoreaction, the antibody conjugate was optionally treated with 4.2 mM H2O2 for 2 hours at room temperature to reoxidize any cysteine thiols that were not modified by attachment to the DBCO moiety. Products of the reaction were evaluated using sodium dodecylsulfate polyacrylamide gel electrophoresis (SDS-PAGE) under non-reducing conditions.

FIG. 4 (4A to 4C) shows results from the SDS-PAGE analysis of pre-immune IgG1 after its modification with DBCO-AZDye™ 647 performed under photo thiol-yne conditions. Results are shown in FIG. 4B (Coomassie Blue stain) and in FIG. 4C (fluorescence in Alexa 647 channel) for duplicate SDS-PAGE gels loaded with molecular weight standards (MW), control IgG1 antibody not treated with reducing agent (IgG (−DTT)), control IgG1 antibody fully reduced by treatment with DTT (IgG (+DTT)), products of reaction of IgG1 with DBCO-AZ647 for 0 (no light), 5, 10 or 15 minutes under irradiation followed by treatment with H2O2 (+H2O2) or without treatment with H2O2 (−H2O2). Duplicate gels are shown in FIG. 4B and FIG. 4C. Locations are indicated for tetrameric antibody (LHHL), dimeric antibody (HL), heavy chain monomer in fully reduced (H*) and disulfide containing (H) state and light chain monomer in fully reduced (L*) and disulfide containing (L) state. IgG1 conjugates having the AZ647 moiety attached via synthetically bridged monomers are indicated by black arrows in FIG. 4C and IgG1 conjugates having the AZ647 moiety but lacking synthetic bridges between monomers are indicated by white arrows in FIG. 4C.

The images in FIG. 4B and FIG. 4C show protein bands (+H2O2) which are assigned as modified species which comprise primarily HL (monovalent antibody) and HLLH (divalent antibody) as marked by black arrows (FIG. 4C). Under no light condition, heavy (H) and light (L) chains also showed some modifications as marked by white arrows (FIG. 4C).

The results demonstrated that reaction of IgG1 antibody with DBCO-AZ647 under irradiation with UVA light yielded antibody conjugates having an AZ647 label moiety and synthetic bridges between antibody subunits that maintained association of tetramers (HLLH) and dimers (HL). Moreover, the results demonstrated that irradiation with light caused a photo thiol-yne reaction, whereas without activation by light the strained DBCO moiety, although capable of attaching to antibody through single cross-linking at low efficiency, was not able to form bridges between antibody subunits.

### Example II

### Modification of IgG1 with Dibenzocyclooctyne-oligonucleotide Modifying Reagent

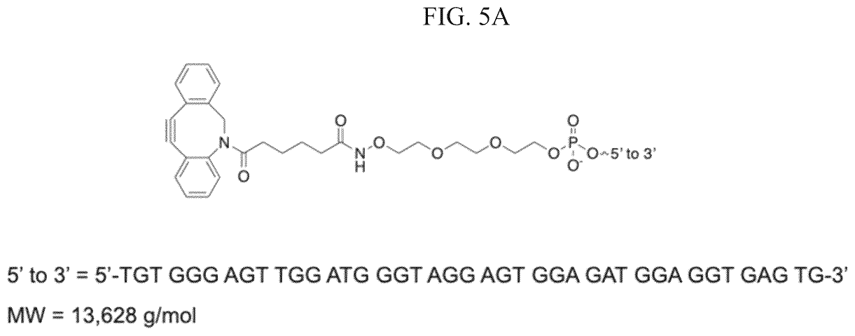

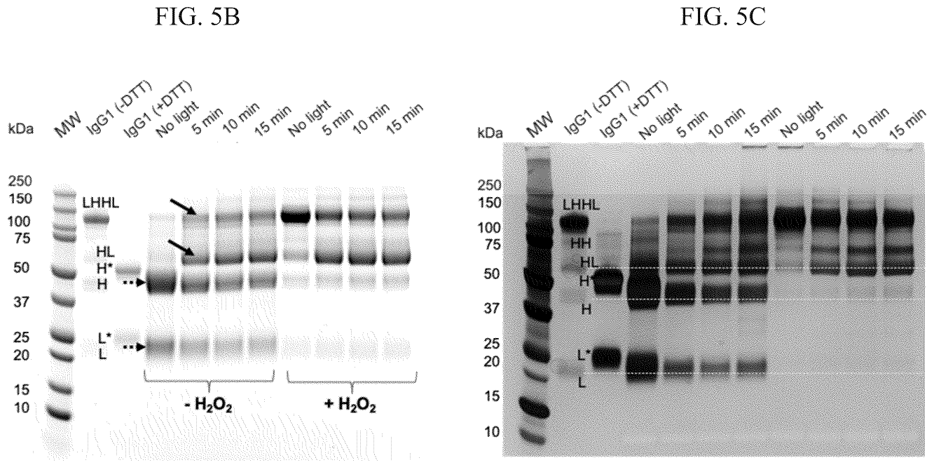

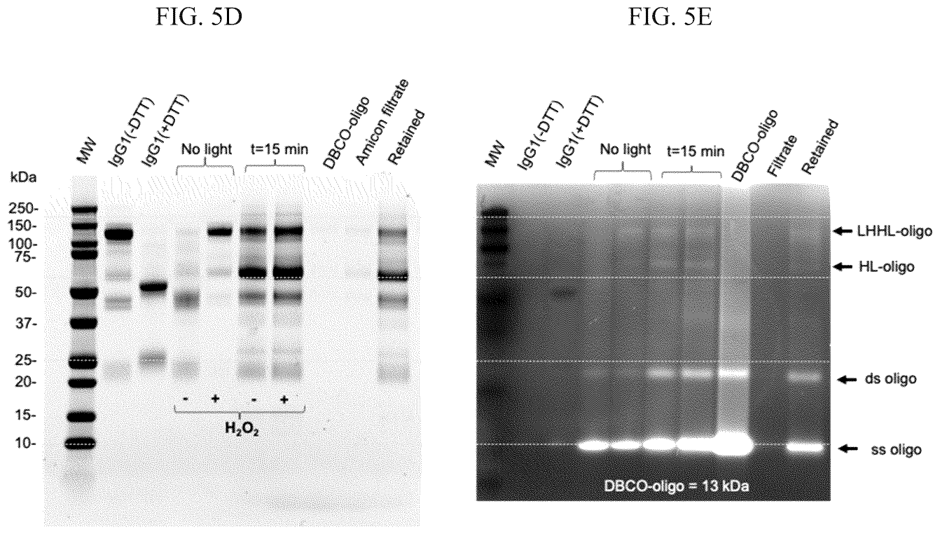

IgG1 antibody (unlabeled rabbit IgG₁, Southern Biotech) dissolved in borate buffered saline (BBS) pH 8.2 (1 mg/mL) was reacted with tenfold molar excess of TCEP at 5° C. for twelve hours to selectively reduce interchain disulfide bonds located at heavy (H)-to-light (L) chains and a heavy (H) chain-to-heavy (H) chain. The reduced IgG1 antibody was then reacted with a modifying reagent having a dibenzocyclooctyne reactive moiety linked to an oligonucleotide via a triethyleneglycol linker moiety (DBCO-TEG-oligo; custom synthesized by Integrated DNA Technologies, Coralville IA; see FIG. 5A) in the presence of 8-fold molar equivalent of azobis(2-methylpropionamidine) dihydrochloride (AAPH; Cat#440914; Sigma-Aldrich) under irradiation with light at 365 nm for variable times between 5 and 15 minutes to produce an antibody conjugate modified to include the oligonucleotide. Following the photoreaction, the antibody conjugate was optionally treated with 4.2 mM H₂O₂ for 2 hours at room temperature to reoxidize any cysteine thiols that were not modified by attachment to the DBCO moiety. Products of the reaction were evaluated using sodium dodecylsulfate polyacrylamide gel electrophoresis (SDS-PAGE) under non-reducing conditions.

The crude mixture was purified through membrane filtration using an Amicon Ultracell centrifugal filter (MWCO 50k; 0.5 mL capacity) and rinsing with PBS pH 7.4. The mixture was centrifuged at 15,000 rpm for 10 min (Eppendorf 5430 R), and its filtrate was collected. This centrifugal filtration was repeated two more times, each using 0.5 mL of PBS pH 7.4 (Gibco).

FIG. 5 (5A to 5E) show results from the SDS-PAGE analysis of IgG₁ after its modification with a dibenzocyclooctyne (DBCO)-tethered oligonucleotide under photo thiol-yne conditions. FIG. 5B shows an SDS-PAGE gel stained with Coomassie Blue and in FIG. 5C shows a duplicate SDS-PAGE stained with silver stain. The duplicate gels were loaded with molecular weight standards (MW), control IgG1 antibody not treated with reducing agent (IgG (−DTT)), control IgG1 antibody reduced by treatment with DTT (IgG (+DTT)), products of reaction of IgG1 with DBCO-TEG-oligo for 0 (no light), 5, 10 or 15 minutes under irradiation followed by treatment with H₂O₂ (+H₂O₂) or without treatment with H₂O₂ (−H₂O₂). Locations are indicated for tetrameric antibody (LHHL), dimeric antibody (HL), heavy chain monomer in fully reduced (H*) and disulfide containing (H) state and light chain monomer in fully reduced (L*) and disulfide containing (L) state. IgG1 conjugates having the oligo moiety attached via synthetically bridged monomers are indicated by solid-line arrows in FIG. 5B and IgG1 conjugates having the oligo moiety but lacking synthetic bridges between monomers are indicated by dashed-line arrows in FIG. 5B. Each image in FIG. 5B and FIG. 5C shows protein bands which are assigned as modified species which include HL (monovalent antibody) and LHHL (divalent antibody) as marked by solid line arrows (FIG. 5B). This result is different from that observed under no light condition in which an intact antibody was found primarily (+H₂O₂).

FIG. 5D shows a second SDS-PAGE gel stained with Coomassie blue to detect protein and FIG. 5E shows the gel stained with SYBR gold to detect nucleic acid. The gel was loaded with molecular weight standards (MW); control IgG1 antibody not treated with reducing agent (IgG (−DTT)); control IgG1 antibody reduced by treatment with DTT (IgG (+DTT)); products of reaction of IgG1 with DBCO-TEG-oligo without light (no light), or with 15 minute irradiation followed by treatment with H₂O₂ (+H₂O₂) or without treatment with H₂O₂ (−H₂O₂); the DBCO-TEG-oligo reagent (DBCO-oligo); the filtrate from Amicon filtration and the retentate from Amicon filtration. Locations are indicated for tetrameric antibody conjugates (LHHL-oligo), dimeric antibody conjugates (HL-oligo), unreacted single stranded oligo (ss oligo) and unreacted double stranded oligo (ds oligo).

The results demonstrated that reaction of IgG1 antibody with DBCO-TEG-oligo under irradiation with UVA light yielded antibody conjugates having the oligo moiety and synthetic bridges between antibody subunits that maintained association of tetramers (LHHL) and dimers (HL). Moreover, the results demonstrated that irradiation with light caused a photo thiol-yne reaction, whereas without activation by light the alkyne moiety, although capable of attaching to antibody, was not able to form bridges between antibody subunits.

### Example III

### Modification of IgG1 with Dibenzocyclooctyne-Polyethylene Glycol Modifying Reagent

IgG1 antibody (unlabeled rabbit IgG₁, Southern Biotech) dissolved in borate buffered saline (BBS) pH 8.2 (1 mg/mL) was reacted with tenfold molar excess of TCEP at 5° C. for 13 hours to produce reduced IgG1 antibody. The reduced IgG1 antibody was then reacted with dibenzocyclooctyne-polyethylene glycol 20k (DBCO-PEG20) in the presence of 8-fold molar equivalent of azobis(2-methylpropionamidine) dihydrochloride (AAPH; Cat#440914; Sigma-Aldrich) under irradiation with light at 365 nm for variable times between 10 and 30 minutes to produce an antibody conjugate modified to include the PEG20 moiety. Following the photoreaction, the antibody conjugate was optionally treated with 4.2 mM H₂O₂ for 1 hour at room temperature to reoxidize any cysteine thiols that were not modified by attachment to the DBCO moiety. Products of the reaction were evaluated using SDS-PAGE under non-reducing conditions.

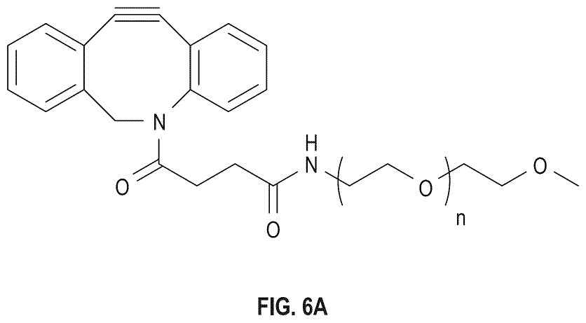

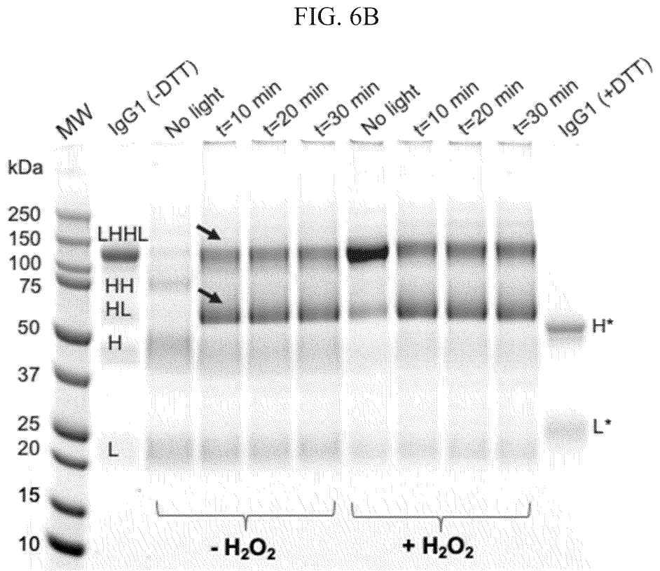

FIG. 6 (6A, 6B) show results from the SDS-PAGE analysis of IgG₁ after its modification with a dibenzocyclooctyne (DBCO)-tethered mPEG under photo thiol-yne conditions. A gel stained with Coomassie blue is shown in FIG. 6. The gel was loaded with molecular weight standards (MW), control IgG1 antibody not treated with reducing agent (IgG (−DTT)), control IgG1 antibody reduced by treatment with DTT (IgG (+DTT)), products of reaction of IgG1 with DBCO-PEG20 for 0 (no light), 10, 20 or 30 minutes under irradiation followed by treatment with H₂O₂ (+H₂O₂) or without treatment with H₂O₂ (−H₂O₂). Locations are indicated for tetrameric antibody (LHHL), dimeric antibody (HL), heavy chain monomer in fully reduced (H*) and disulfide containing (H) state and light chain monomer in fully reduced (L*) and disulfide containing (L) state. IgG1 conjugates having the PEG20 moiety attached via synthetically bridged monomers are indicated by arrows in FIG. 6.

The results demonstrated that reaction of IgG1 antibody with DBCO-PEG20 under irradiation with UVA light yielded antibody conjugates having a PEG20 moiety and synthetic bridges between antibody subunits that maintained association of tetramers (LHHL) and dimers (HL). Moreover, the results demonstrated that irradiation with light caused a photo thiol-yne reaction, whereas without activation by light the alkyne moiety was not able to form bridges between antibody subunits.

### Example IV

### Modification of IgG1 with Propargyl-PEG-Biotin Modifying Reagent

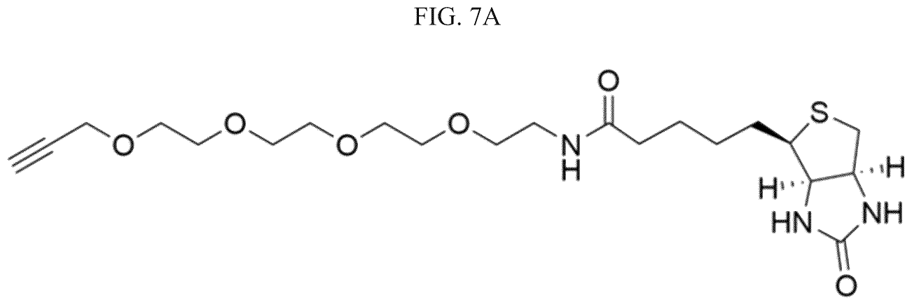

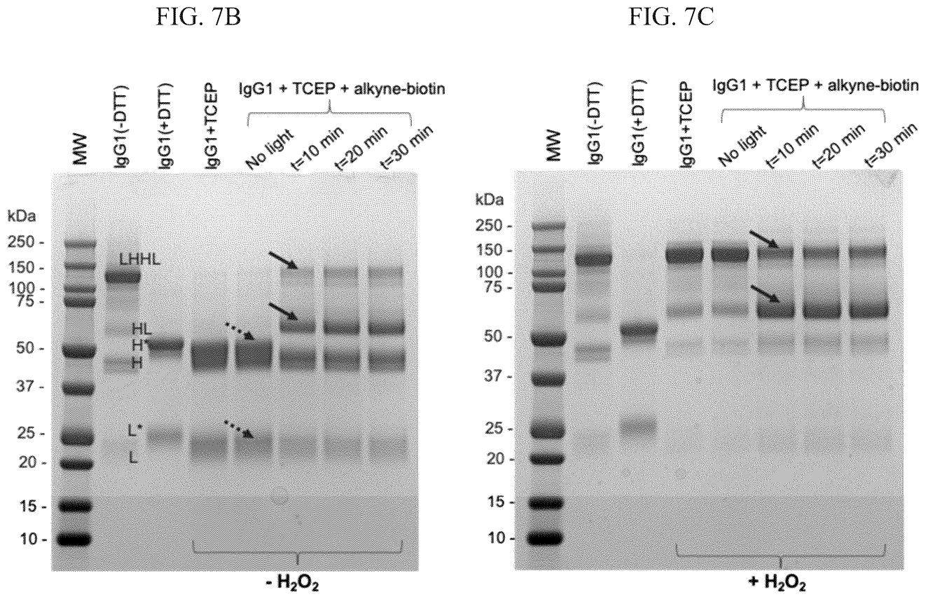

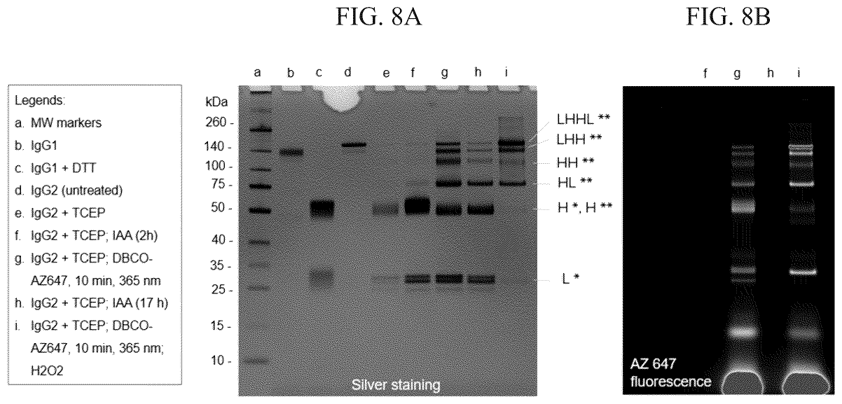

IgG1 antibody (unlabeled rabbit IgG₁, Southern Biotech dissolved in borate buffered saline (BBS) pH 8.2 (1 mg/mL) was reacted with tenfold molar excess of TCEP at room temperature for 14 hours to produce reduced IgG1 antibody. The reduced IgG1 antibody was then reacted with 10 molar equivalents of Propargyl-PEG₄-biotin (Click Chemistry Tools, Cat # TA105-5, FIG. 7A) and 8 molar equivalents of 2,2′-azobis-2-methyl-propanimidamide, dihydrochloride (AAPH) under irradiation with light at 365 nm for variable times between 10 and 30 minutes to produce an antibody conjugate modified to include the PEG₄-biotin moiety. Following the photoreaction, the antibody conjugate was optionally treated with 4.2 mM H₂O₂ for 2 hours at room temperature to reoxidize any cysteine thiols that were not modified by attachment to the DBCO moiety. Products of the reaction were evaluated using SDS-PAGE under non-reducing conditions.

FIG. 7 (7B, 7C) show results from the SDS-PAGE analysis of IgG₁ after its modification with a propargyl-PEG₄-biotin under photo thiol-yne conditions. FIG. 7B shows a gel stained with Coomassie blue and loaded with molecular weight standards (MW), control IgG1 antibody not treated with reducing agent (IgG (−DTT)), control IgG1 antibody reduced by treatment with DTT (IgG (+DTT)), IgG1 antibody treated with TCEP, products of reaction of IgG1 with TCEP followed by Propargyl-PEG₄-biotin for 0 (no light), 10, 20 or 30 minutes under irradiation without treatment with H₂O₂ (−H₂O₂). FIG. 7C shows a gel stained with Coomassie blue and loaded with molecular weight standards (MW), control IgG1 antibody not treated with reducing agent (IgG (−DTT)), control IgG1 antibody reduced by treatment with DTT (IgG (+DTT)), IgG1 antibody treated with TCEP, products of reaction of IgG1 with TCEP followed by Propargyl-PEG₄-biotin for 0 (no light), 10, 20 or 30 minutes under irradiation with treatment with H₂O₂ (+H₂O₂). Locations are indicated for tetrameric antibody (LHHL), dimeric antibody (HL), heavy chain monomer in fully reduced (H*) and disulfide containing (H) state and light chain monomer in fully reduced (L*) and disulfide containing (L) state. IgG1 conjugates having the PEG₄ biotin moiety attached via synthetically bridged monomers are indicated by solid arrows in FIG. 7B and FIG. 7C. IgG1 conjugates having the PEG₄ biotin moiety attached absent synthetically bridged monomers are indicated by dashed arrows in FIG. 7B.

The results demonstrated that reaction of IgG1 antibody with Propargyl-PEG₄-biotin under irradiation with UVA light yielded antibody conjugates having a PEG 20 moiety and synthetic bridges between antibody subunits that maintained association of tetramers (LHHL) and dimers (HL). Moreover, the results demonstrated that irradiation with light caused a photo thiol-yne reaction, whereas without activation by light the alkyne moiety was not able to form bridges between antibody subunits.

### Example V

### Modification of Anti-tau 2N IgG₂ with Dibenzocyclooctyne-AZDye™ 647 Modifying Reagent

Anti-tau 2N IgG₂ antibody (Biolegend, Cat# SIG-39408) dissolved in phosphate buffered saline (PBS) pH 7.2 (2 mg/mL) was reacted with tenfold molar excess of TCEP at 5° C. for overnight or 12 hours to produce reduced IgG2 antibody. The reduced IgG2 antibody was then reacted with DBCO-AZ647 (Click Chemistry Tools, Cat# 1302; see FIG. 4A) in the presence of 8-fold molar equivalent of AAPH (Cat#440914; Sigma-Aldrich) under irradiation with light at 365 nm for 10 minutes to produce an antibody conjugate modified to include the AZDye™ 647 (lex=648 nm, lem=671 nm). Following the photoreaction, the antibody conjugate was optionally treated with 4.2 mM H2O2 for 2 hours at room temperature to reoxidize any cysteine thiols that were not modified by attachment to the DBCO moiety. Products of the reaction were evaluated using SDS-PAGE under non-reducing conditions.

Results are shown in FIG. 8A (silver stain) and in FIG. 8B (fluorescence in Alexa 647 channel) for the SDS-PAGE (FIG. 8A) loaded with molecular weight standards (lane a), IgG1 (untreated; lane b), IgG1+DTT (lane c), IgG2 (untreated; lane d), IgG2+TCEP (lane e), IgG2+TCEP; IAA, 2h (lane f), IgG2+TCEP; DBCO-AZ647, 10 min, 365 nm (lane g), IgG2+TCEP; IAA, 17 h (lane h), IgG2+TCEP; DBCO-AZ647, 10 min, 365 nm; H202 (lane i). Locations are indicated for tetrameric antibody (LHHL), dimeric antibody (HL), heavy chain (H), light chain (L) and modified antibody species associated with them, each marked as ** (bridged) or * (crosslinked). Anti-tau 2N IgG2 conjugates having the AZ647 moiety attached via a synthetic bridge or a crosslink are indicated by fluorescence in FIG. 8B. These results indicated that anti-tau 2N IgG2 antibody reacted with DBCO-AZ647 under irradiation with UVA light and yielded its conjugates having an AZ647 label moiety and synthetic bridges between antibody subunits that maintained association of tetramers (LHHL) and dimers (HL).

The results demonstrated that interchain disulfide bonds of anti-tau 2N IgG2 were selectively reduced by treatment with TCEP and the resulting sulfhydryls were able to react with DBCO-AZDye™ 647 in the presence AAPH under long wavelength ultraviolet radiation to produce anti-tau 2N IgG₂ labeled with AZDye™ 647. Subsequent treatment with hydrogen peroxide resulted in reoxidation of unreacted sulfhydryls back to their original disulfide bonds.

### Example VI

### Modification of Anti-YWL IgG₁ with Dibenzocyclooctyne-AZDye™ 647 Modifying Reagent

Anti-YWL IgG₁ antibody (Nautilus Biotechnology, San Carlos, CA) dissolved in phosphate buffered saline (PBS) pH 7.2 (1 mg/mL) was reacted with tenfold molar excess of TCEP at 5° C. for overnight or 12 hours to produce reduced IgG₁ antibody. The reduced IgG₁ antibody was then reacted with DBCO-AZ647 (Click Chemistry Tools, Cat# 1302; see FIG. 4A) in the presence of 8-fold molar equivalent of AAPH (Cat#440914; Sigma-Aldrich) under irradiation with light at 365 nm for 10 minutes to produce an antibody conjugate modified to include the AZDye™ 647 (lₑₓ=648 nm, lₑₘ=671 nm). Following the photoreaction, the antibody conjugate was optionally treated with 4.2 mM H₂O₂ for 2 hours at room temperature to reoxidize any cysteine thiols that were not modified by attachment to the DBCO moiety. Products of the reaction were evaluated using SDS-PAGE under non-reducing conditions.

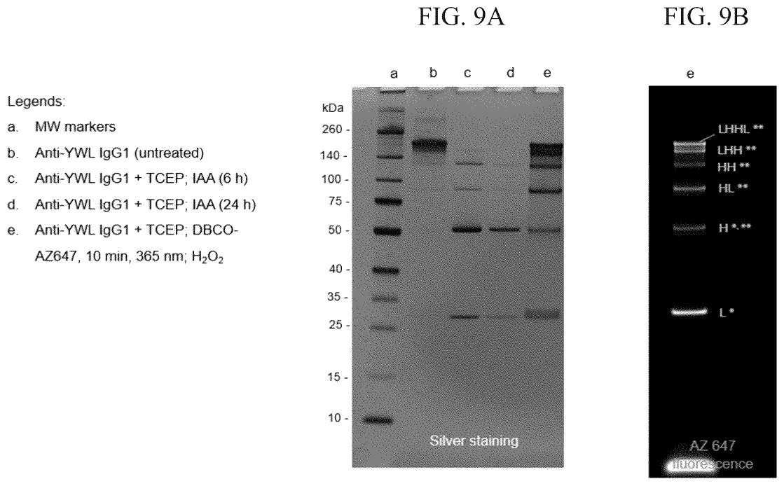

Results are shown in FIG. 9A (silver stain) and in FIG. 9B (fluorescence in Alexa 647 channel) for the SDS-PAGE (FIG. 9A) loaded with molecular weight standards (lane a), anti-YWL IgG₁ (untreated; lane b), anti-YWL IgG₁+TCEP; IAA, 6 h (lane c), anti-YWL IgG₁+TCEP; IAA, 24 h (lane d), and anti-YWL IgG₁+TCEP; DBCO-AZ647, 10 min, 365 nm; H2O2 (lane e). Locations are indicated for tetrameric antibody (LHHL), dimeric antibody (HL), heavy chain (H), light chain (L) and modified antibody species associated with them, each marked as ** (bridged) or * (crosslinked). Anti-YWL IgG₁ conjugates having the AZ647 moiety attached via a synthetic bridge or a crosslink are indicated by fluorescence in FIG. 9B.

These results demonstrated that anti-YWL IgG₁ antibody reacted with DBCO-AZ647 under irradiation with UVA light and yielded its conjugates having an AZ647 label moiety and synthetic bridges between antibody subunits that maintained association of tetramers (LHHL) and dimers (HL).

### Example VII

### Modification of Anti-WNK IgG₁ with Dibenzocyclooctyne-AZDye™ 647 Modifying Reagent

Anti-WNK IgG1 antibody (Nautilus Biotechnology, San Carlos, CA) dissolved in phosphate buffered saline (PBS) pH 7.2 (1 mg/mL) was reacted with tenfold molar excess of TCEP at 5° C. for overnight or 12 hours to produce reduced IgG₁ antibody. The reduced IgG₁ antibody was then reacted with DBCO-AZ647 (Click Chemistry Tools, Cat# 1302; see FIG. 4A) in the presence of 8-fold molar equivalent of AAPH (Cat#440914; Sigma-Aldrich) under irradiation with light at 365 nm for 10 minutes to produce an antibody conjugate modified to include the AZDye™ 647 (lex=648 nm, lem=671 nm). Following the photoreaction, the antibody conjugate was optionally treated with 4.2 mM H2O2 for 2 hours at room temperature to reoxidize any cysteine thiols that were not modified by attachment to the DBCO moiety. Products of the reaction were evaluated using SDS-PAGE under non-reducing conditions.

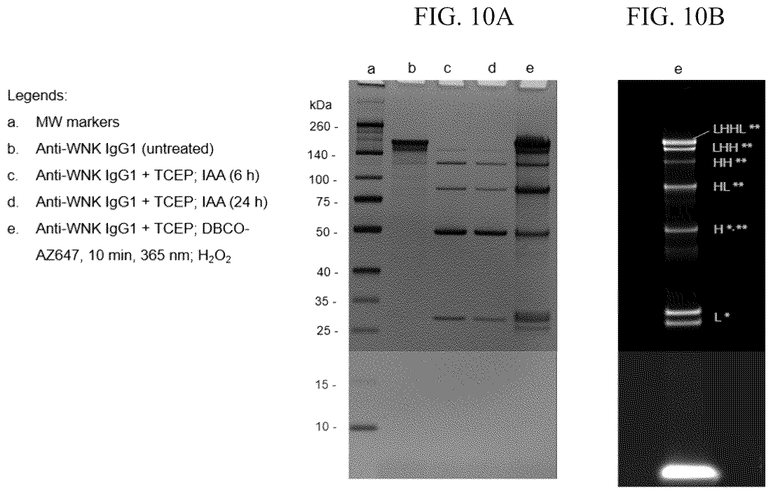

Results are shown in FIG. 10A (silver stain) and in FIG. 10B (fluorescence in Alexa 647 channel) for the SDS-PAGE loaded with molecular weight standards (lane a), anti-WNK IgG1 (untreated; lane b), anti-WNK IgG1+TCEP; IAA, 6 h (lane c), anti-WNK IgG1+TCEP; IAA, 24 h (lane d), and anti-WNK IgG1+TCEP; DBCO-AZ647, 10 min, 365 nm; H2O2 (lane e). Locations are indicated for tetrameric antibody (LHHL), dimeric antibody (HL), heavy chain (H), light chain (L) and modified antibody species associated with them, each marked as ** (bridged) or * (crosslinked). Anti-WNK IgG1 conjugates having the AZ647 moiety attached via a synthetic bridge or a crosslink are indicated by fluorescence in FIG. 10B.

These results demonstrated that anti-WNK IgG1 antibody reacted with DBCO-AZ647 under irradiation with UVA light and yielded its conjugates having an AZ647 label moiety and synthetic bridges between antibody subunits that maintained association of tetramers (LHHL) and dimers (HL).

### Example VIII

### Modification of IgG₁ with Polyethyleneimine(propargyl)ₙ₌₁₀(azide)ₘ₌₁₀(AZ488)_{o=0.05} Modifying Reagent

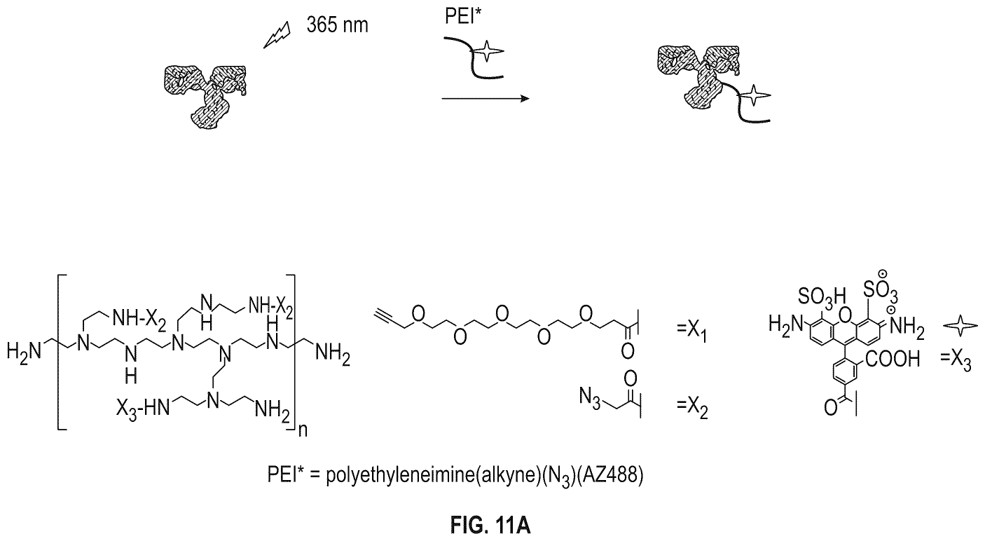

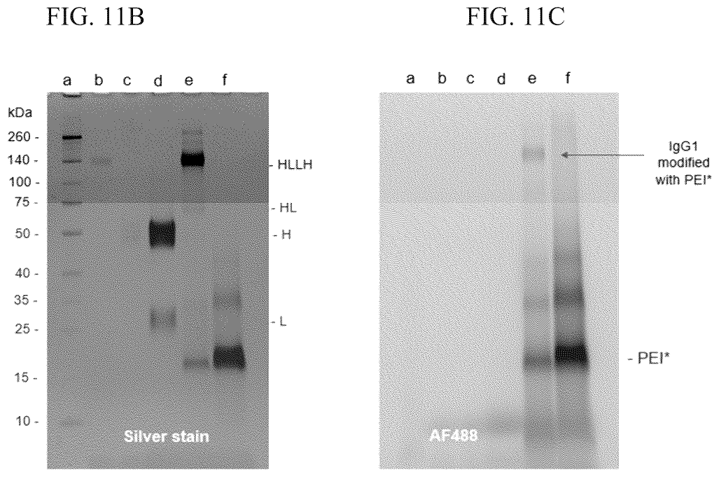

IgG1 antibody (unlabeled rabbit IgG₁, Southern Biotech) dissolved in borate buffered saline (BBS) pH 8.2 (1 mg/mL) was reacted with tenfold molar excess of TCEP at 5° C. for overnight or 12 hours to produce reduced IgG1 antibody. The reduced IgG1 antibody was then reacted with polyethyleneimine(propargyl)ₙ₌₁₀(azide)ₘ₌₁₀(AF488)_{o=0.05} (see FIG. 11A) in the presence of 8-fold molar equivalent of AAPH (Cat#440914; Sigma-Aldrich) under irradiation with light at 365 nm for 15 minutes to produce an antibody conjugate modified to include the PEI(propargyl)(azide)(AZ488). Following the photoreaction, the antibody conjugate was optionally treated with 4.2 mM H₂O₂ for 2 hours at room temperature to reoxidize any cysteine thiols that were not modified by attachment to the PEI moiety. This modified antibody solution was concentrated using a membrane filtration tube (Amicon ultra-0.5 mL; molecular weight cutoff size=50k) at 8000 rpm for 5 min (Eppendorf 5430/5430R). This membrane filtration was repeated three times by replenishing 0.2 mL of phosphate buffered saline (PBS), pH 7.2 and spinning at the same rate as stated. Finally, its solution was recovered by centrifugation at 5000 rpm for 3 min and adjusted to a final volume of 50 microL using PBS pH 7.2. Products of the reaction were evaluated using SDS-PAGE under non-reducing conditions.

Results are shown in FIG. 11B (silver stain) and in FIG. 11C (fluorescence in Alexa 488 channel) for the SDS-PAGE (FIG. 11B) loaded with molecular weight standards (lane a), IgG₁ (untreated; lane b), IgG₁+DTT (lane c); IgG₁+TCEP (lane d); IgG₁+TCEP; PEI(propargyl)(azide)(AZ488), 365 nm, 10 min; H₂O₂ (crude reaction mixture; lane e) and PEI(propargyl)(azide)(AZ488) (lane f).

The results demonstrate selective reduction of interchain disulfide bonds by treatment with TCEP and reaction of the resulting sulfhydryls with polyethyleneimine(propargyl)ₙ₌₁₀(azide)ₘ₌₁₀(AF488)_{o=0.05} in the presence of AAPH under irradiation of long wavelength ultraviolet light for 15 min. After irradiation, the PEI-modified antibody was treated with hydrogen peroxide (+H₂O₂) for the reoxidation of unreacted sulfhydryls back to their original disulfide bonds.

### Example IX

### Click Conjugation of DBCO-AZ647 with IgG₁ Pre-Modified with Polyethyleneimine(propargyl)ₙ₌₁₀(azide)ₘ₌₁₀(AF488)_{o=0.05}

To a vial containing a solution of IgG₁ pre-modified with PEI* (see EXAMPLE VIII) was added a solution of DBCO-AZ647 (FIG. 4A; 10 or 20 molar equivalent to the modified IgG₁). This solution was left at ambient temperature for 15 hours. Products of the reaction were evaluated using SDS-PAGE under non-reducing conditions. The gel was loaded with molecular weight standards (lane a), IgG1 (untreated; lane b), IgG1+DTT (lane c); IgG1+TCEP (lane d); IgG1 pre-modified with PEI(propargyl)(azide)(AZ488) (lane e), IgG1 pre-modified with PEI(propargyl)(azide)(AZ488)+DBCO-AZ647 (10 molar equivalent) (lane f), IgG1 pre-modified with PEI(propargyl)(azide)(AZ488) (lane g)+DBCO-AZ647 (20 molar equivalent) and PEI(propargyl)(azide)(AZ488) (lane h).

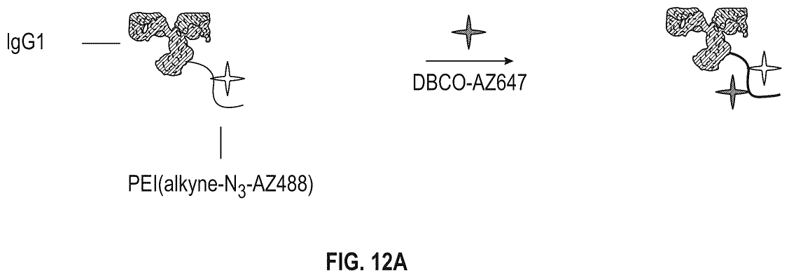

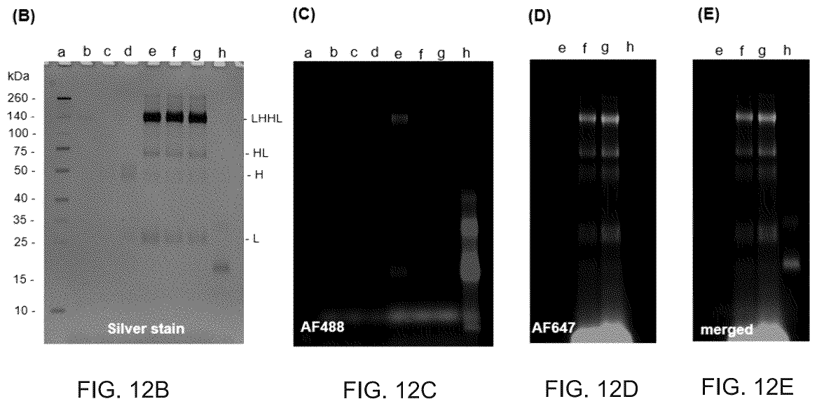

FIG. 12A shows a schematic for strain-promoted azide-alkyne click (SPAAC) reaction of PEI*(azide)-presenting IgG1 with DBCO-AZ647. Results are shown in FIG. 12B (silver stain), FIG. 12C (fluorescence in Alexa 488 channel), FIG. 12D (fluorescence in Alexa 647 channel) and FIG. 12E (merged of two fluorescent images each at AF488 and AF647) for the SDS-PAGE (FIG. 12B).

The results demonstrated that IgG1 attached with azide (through its PEI* modification) reacted with DBCO-AZ647 under a strain-promoted azide-alkyne click (SPAAC) condition. The image in each of FIG. 12B to FIG. 12E shows protein bands (lanes f, g) which are assigned as AZ647 conjugated species. Most of these bands which include primarily LHHL along with HL, H and L species showed such dye conjugation through strong fluorescence at 647 nm.

### Example X

### Binding Assays

This example demonstrates binding of modified antibodies to target epitopes.

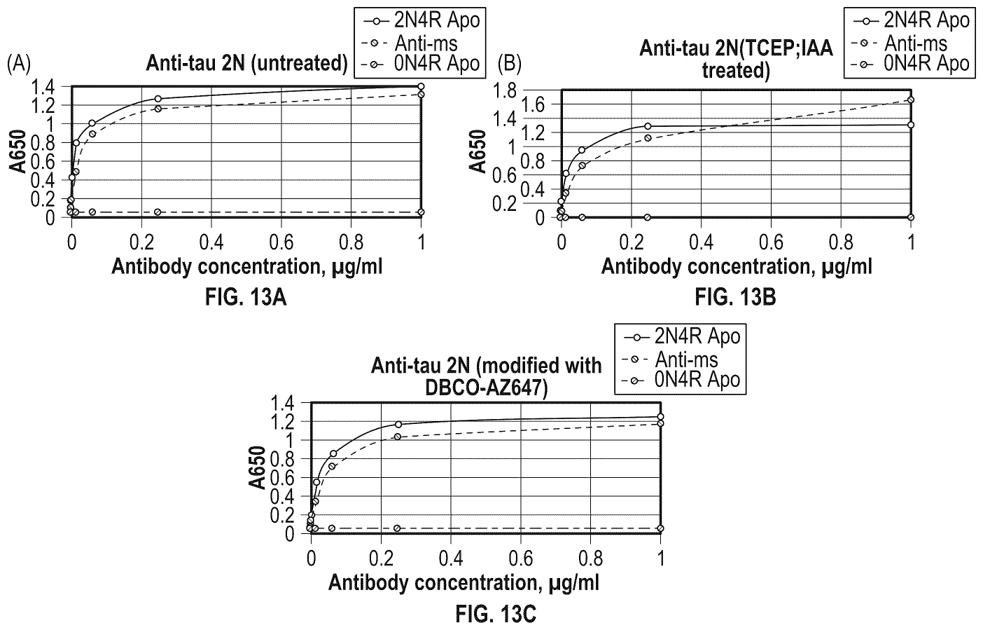

Three species of anti-tau 2N were obtained as set forth in Example V and then subjected to enzyme-linked immunosorbent assay (ELISA) to measure binding to two different target antigens (2N4R apo, anti-ms) and a negative control (0N4R apo). As shown in FIG. 13A, unmodified anti-tau 2N (as characterized in lane d of FIG. 8A) had comparable binding to 2N4R (reaching an A650 of about 1.4 at an antibody concentration of 1 μg/ml) and anti-ms (reaching an A650 of about 1.3 at an antibody concentration of 1 μg/ml), but did not appreciably bind to the 0N4R negative control, even at an antibody concentration of 1 μg/ml). As shown in FIG. 13B, Anti-tau 2N that had been treated with TCEP and IAA (as characterized in lane h of FIG. 8A) had comparable binding to 2N4R (reaching an A650 of about 1.3 at an antibody concentration of 1 μg/ml) and anti-ms (reaching an A650 of about 1.6 at an antibody concentration of 1 μg/ml), but did not appreciably bind to the 0N4R negative control, even at an antibody concentration of 1 μg/ml). As shown in FIG. 13C, Anti-tau 2N modified with DBCO-AZ647 (as characterized in lane i of FIG. 8A) had comparable binding to 2N4R (reaching an A650 of about 1.25 at an antibody concentration of 1 μg/ml) and anti-ms (reaching an A650 of about 1.1.15 at an antibody concentration of 1 μg/ml), but did not appreciably bind to the 0N4R negative control, even at an antibody concentration of 1 μg/ml). These results demonstrated that treatment of the anti-tau 2N antibody with TCEP, IAA and DBCO-AZ647 did not adversely impact affinity or specificity of binding between the antibody and its known target epitopes.

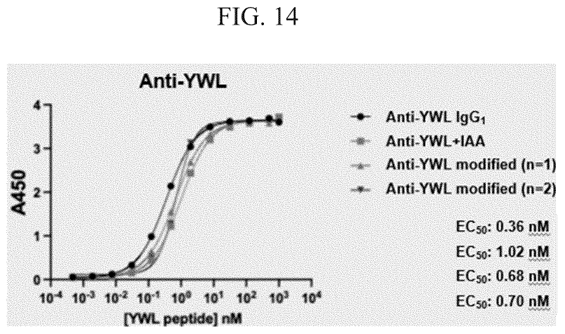

Three species of anti-YWL IgG₁ were obtained as set forth in Example VI and then subjected to enzyme-linked immunosorbent assay (ELISA) to measure binding to peptide YWL. As shown in FIG. 14, unmodified anti-YWL IgG₁ (as characterized in lane b of FIG. 9A) had an EC₅₀ of 0.36 nM, anti-YWL IgG₁ treated with IAA (as characterized in lane d of FIG. 9A) had an EC₅₀ of 1.02 nM, and two samples of anti-YWL IgG₁ modified with DBCO-AZ647 (as characterized in lane e of FIG. 9A) had EC₅₀ of 0.68 nM and 0.70 nM, respectively. These results demonstrated that treatment of the anti-YWL IgG₁ antibody with IAA and DBCO-AZ647 did not adversely impact affinity of the antibody for its known target epitope.

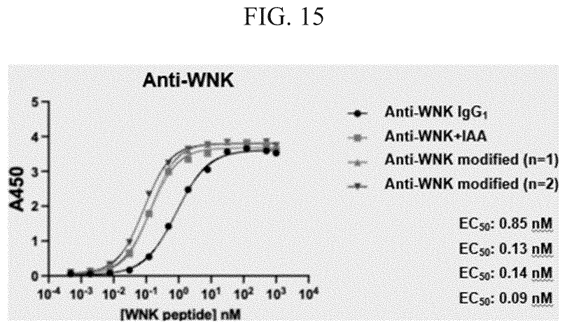

Three species of anti-WNK IgG₁ were obtained as set forth in Example VII and then subjected to enzyme-linked immunosorbent assay (ELISA) to measure binding to peptide WNK. As shown in FIG. 15, unmodified anti-WNK IgG₁ (as characterized in lane b of FIG. 10A) had an EC₅₀ of 0.85 nM, anti-WNK IgG₁ treated with IAA (as characterized in lane d of FIG. 10A) had an EC₅₀ of 0.13 nM, and two samples of anti-WNK IgG₁ modified with DBCO-AZ647 (as characterized in lane e of FIG. 10A) had EC₅₀ of 0.14 nM and 0.09 nM, respectively. These results demonstrated that treatment of the anti-WNK IgG₁ antibody with IAA and DBCO-AZ647 did not adversely impact affinity of the antibody for its known target epitope.

### Example XI

### Synthesis of Propargyl-PEG₇-Azide

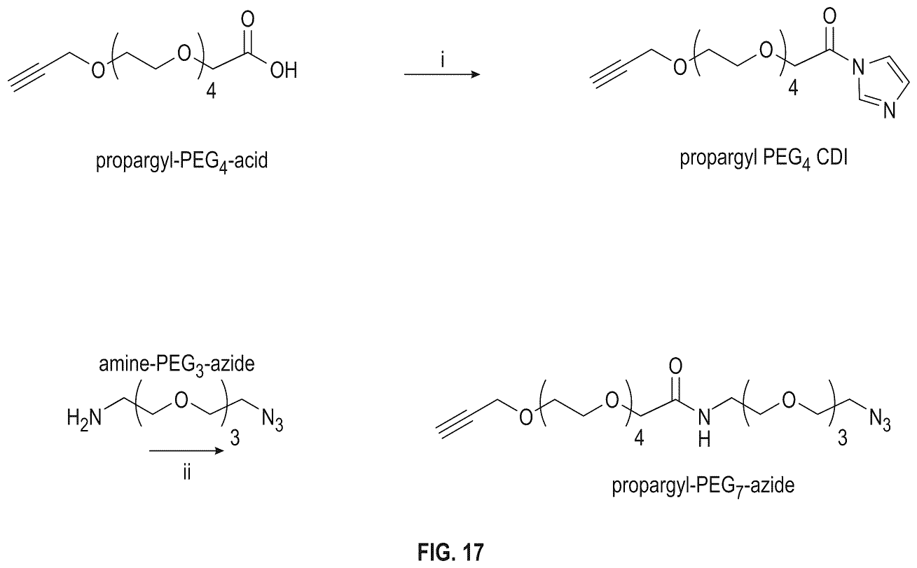

To a glass vial loaded with propargyl-PEG₄ acid (15 mg, 49.3 μmol) was added anhydrous acetonitrile (0.2 mL). After its solubilization, this carboxylic acid was activated to an amine-reactive functionality by treatment with carbonyldiimidazole (CDI; 9.6 mg, 59.1 μmol) which was added as a solution in anhydrous acetonitrile (0.1 mL). After this CDI addition, this mixture was stirred at room temperature for 2 h prior to adding amine-PEG₃-azide (11.4 mg, 52.2 μmol; 1.1 eq) dissolved in acetonitrile (0.1 mL). This reaction mixture was stirred at room temperature while its reaction progress was monitored by TLC analysis in a silica-coated glass plate by eluting with 5% methanol in dichloromethane. After 3 h, its reaction appeared almost completed given the TLC analysis with UV lamp illumination and ninhydrin staining showing that a new product band running at retention factor (R_{f})=0.3 formed with only a small amount of each reactant left. For product purification, the mixture was concentrated using CentriVap™ and its residue was purified by CombiFlash™ chromatography: eluent=methanol in dichloromethane (isocratic: 0-1 min at 3% methanol; linear gradient: 1-7 min at 3% to 100% methanol); flow rate=30 mL/min. The product, propargyl-PEG₇-azide, was obtained as a colorless oily residue. FIG. 17 shows a diagram of the synthesis of propargyl-PEG₇-azide.

### Example XII

### Synthesis of Propargyl-PEG₁₂-Azide

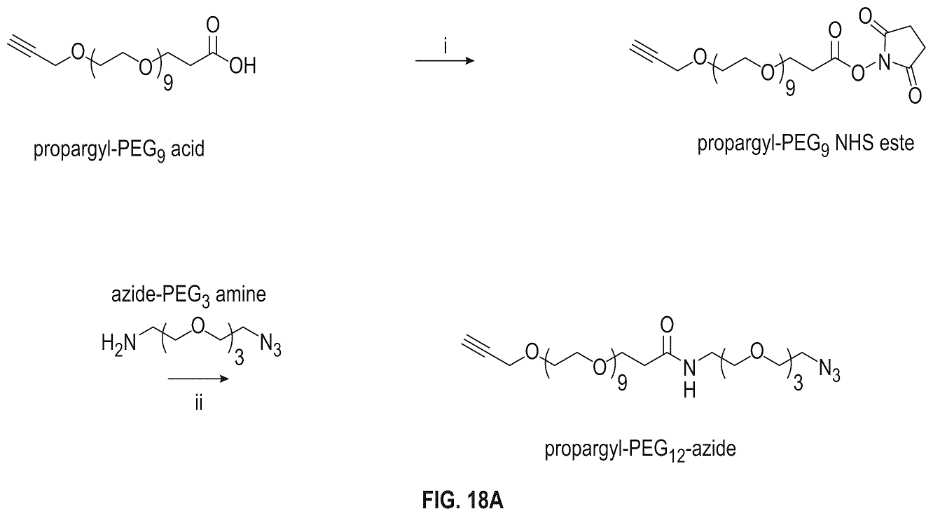

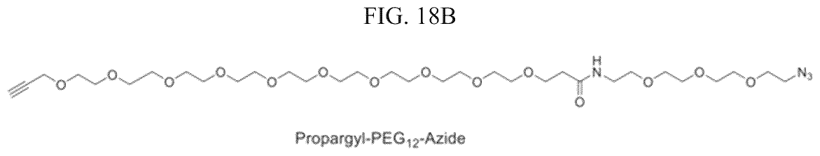

In a vial was loaded N-hydroxysuccinimide (NHS; 2.9 mg, 25 mmol) and N-ethyl-N′-(3-dimethylaminopropyl)carbodiimide hydrochloride (EDC; 4.8 mg, 25 mmol). To this mixture was added propargyl-PEG₉ acid (11 mg, 21 μmol) dissolved in anhydrous DMSO (50 μL). This reaction mixture was shaken at 25° C. to generate its activated NHS ester. After 16 h of activation, it was added to a vial pre-loaded with azide-PEG₃ amine (6.9 mg, 31 mmol; 1.5 eq to alkyne) and N,N-diisopropyl-N-ethylamine (DIPEA) (5.5 μL, 32 mmol). After mixing well, the vial was shaken at 25° C. while its reaction progress was monitored by TLC analysis in a silica-coated glass plate eluting with 10% methanol in dichloromethane. A TLC analysis performed after 6 h indicated a new, major band with a retention factor (R_{f}) of 0.33 with only a small amount of each reactant left. For product purification, it was processed by evaporating volatiles using CentriVap™ and its residue was diluted with acetonitrile to a volume of 0.2 mL. It was then mixed with 0.6 mL of saturated sodium bicarbonate solution (pH 9.5) prior to performing product extraction with dichloromethane (4×0.6 mL). All organic layers were pooled and evaporated using CentriVap™, which afforded the desired product, propargyl-PEG₁₂-azide, as a colorless syrup. Its stock solution was prepared by adding 0.5 mL of acetonitrile (0.42 mM) and used without further treatment. FIG. 18A shows a diagram of a synthesis of propargyl-PEG₁₂-azide and FIG. 18B shows the chemical structure for propargyl-PEG₁₂-azide.

### Example XIII

### Screening of Light Wavelengths in Photolabeling of Cell Lysate Proteins with DBCO-AZ647

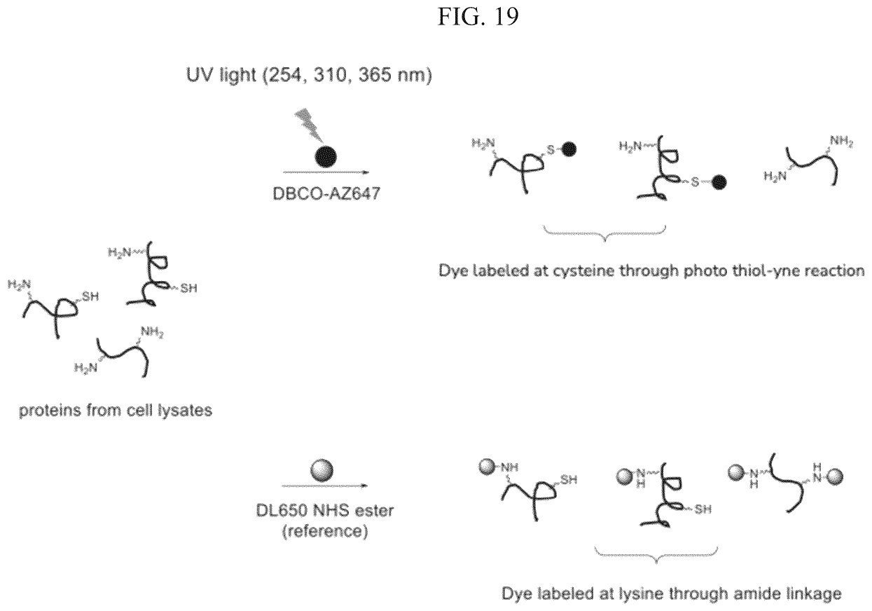

This example describes screening and identification of optimal wavelengths of light applicable in photo thiol-yne chemistry determined by using protein mixtures present in cell lysates. In this example as depicted in FIG. 19, each protein sample is treated with DBCO-PEG₄-mTz and exposed to a light source at either 254 nm, 310 nm, or 365 nm to enable their photoreaction at cysteine residues. Their efficiency of mTz addition is assessed by click conjugation with TCO-Cy5 dye through Inverse Electron Demand Diels-Alder (IEDDA). Comparison of their fluorescent intensities suggests irradiation at 365 nm appears the most effective among those three wavelengths.

First, proteins obtained from K562 cell lysates (0.2 nmol, 0.514 mg/mL 5% SDS PBS pH 8) was treated with a TCEP solution (4 μL, 3.49 mM) for reducing disulfide bonds to free cysteine thiols. This mixture was shaken at 37° C. for 1 h prior to treatment with DBCO-AZ647 (5.2 nmol, 0.94 mM; 50 eq) and AAPH (5.6 μL, 3.7 mM; 1000 eq). This final mixture was incubated for 10 min and divided into four aliquots equally. Their irradiation was performed in Asahi Spectra Max-350 equipped with filters that permit illumination at certain wavelengths of light. Each of three aliquots was exposed to light at 254 nm, 310 nm or 365 for 7 min while the fourth one remained not exposed to light to serve a control. After irradiation, each solution was characterized by SDS-PAGE gel analysis.

Protein modifications were analyzed by sodium dodecylsulfate (SDS)-polyacrylamide gel electrophoresis (PAGE). It was performed in a mini cassette electrophoresis kit using pre-casted Bolt™ Bis-Tris Plus polyacrylamide gels (4-12%; Invitrogen by Thermo Scientific) and a MEPS running buffer. Each sample was prepared in a sample loading buffer (Bolt™, 1×lithium dodecylsulfate (LDS) sample buffer; Novex) and loaded onto the SDS-PAGE gel. Gels were loaded in a mini gel tank apparatus (Bio-Rad) and electrophoresis was performed at constant 200 V for 22 min (room temperature). After electrophoresis, each gel was removed from its plastic cassette, rinsed with water, and photographed and/or imaged for fluorescent bands (ChemiDoc™ MP imaging system; Bio-Rad). After imaging, the gel was fixed for 10 min in 10% acetic acid, 30% ethanol in water, treated for silver staining using the Pierce™ silver stain kit (Thermo Scientific) according to its instruction and proceeded to its imaging (Bio-Rad).

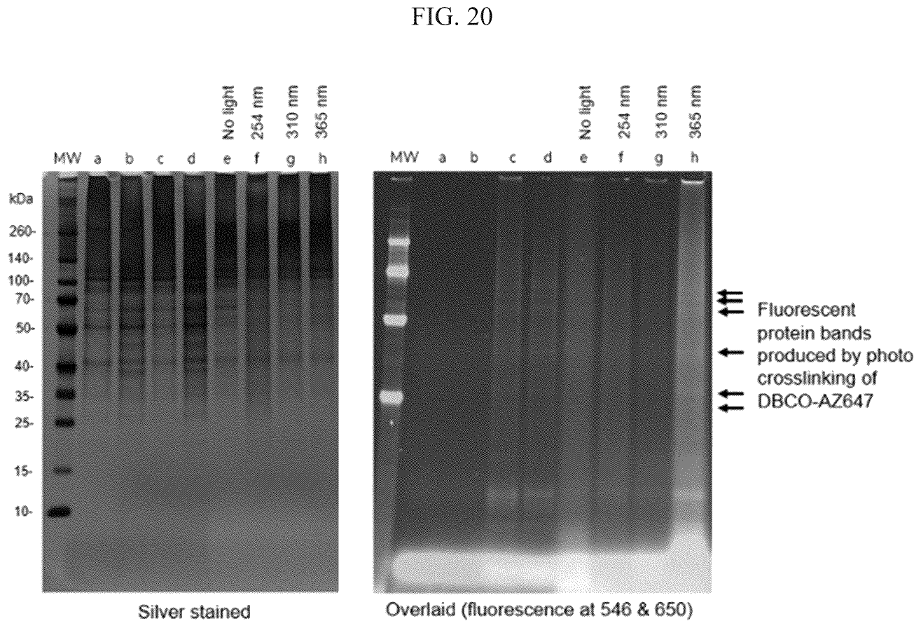

FIG. 20 shows photographs of a gel after silver staining (left) or fluorescent detection at 546 nm and 650 nm). Lane a-b: lysate proteins (a); lysate proteins+TCEP (b); lane c-d: lysate proteins+DL650 NHS ester (50 eq, c); lysate proteins+TCEP+DL650 NHS ester (50 eq, d); lane e-h (lysate proteins+TCEP+DBCO-AZ647): no irradiation (e); 254 nm, 7 min (f); 310 nm, 7 min (g); 365 nm, 7 min (h). The gel image on the right in FIG. 20 (647+593 channel) shows a group of fluorescent protein bands as pointed by black arrows. These are supportive of the occurrence of protein modification through photo thiol-yne reaction. Their fluorescent intensity is variable by light wavelengths. Of three wavelengths, fluorescent bands acquired from 365 nm irradiation appear more intense than others from 254 nm or 310 nm. This suggests 365 nm light as a robust light source in photo thiol-yne modification of proteins.

### Example XIV

### Photoreaction of Cell Lysate Proteins with DBCO-PEG₄-mTz

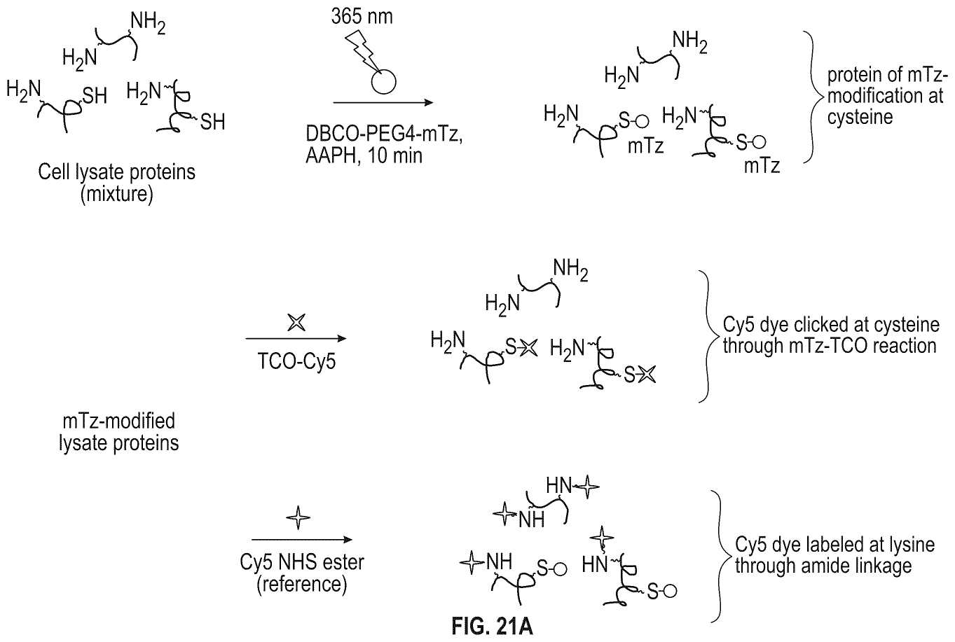

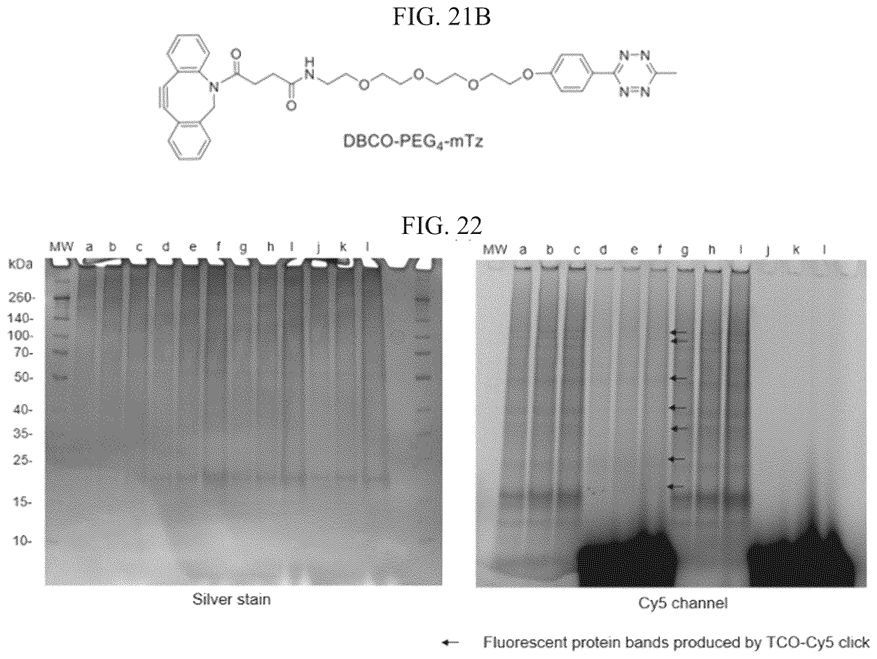

This example describes a representative method for mTz modification of proteins present in cell lysates through photo thiol-yne chemistry. In this example, proteins are modified by photoreaction with methyl tetrazine (mTz) and those mTz-modified proteins are characterized by click conjugation with TCO-dye through Inverse Electron Demand Diels-Alder (IEDDA) reaction. A diagram of the reaction scheme is shown in FIG. 21A.

First, a lysate protein sample obtained from K562 cells (50 μL, 0.514 mg/mL in 5% SDS PBS pH 8) was loaded in an Eppendorf vial and treated with a TCEP solution (12 mL; 2.5 mM). After incubation at room temperature for 2 h, a fraction of the TCEP-treated solution (50 mL) was aliquoted in a separate vial and mixed with AAPH (19 mL, 5 mg/mL PBS; 200 eq) and DBCO-PEG₄-mTz (23 mL; 200 eq; FIG. 21B). This mixture was irradiated at 365 nm for 10 min in Asahi Spectra Max-350, and then treated with iodoacetamide (IAA) (8.7 mL, 10 mM) for blocking any remaining thiols through S-alkylation. After shaking (650 rpm) at 25° C. for 30 min, excess reagents in the mixture were removed through acetone precipitation. For this, this solution was divided equally in two aliquots, each 17 mL, in Eppendorf vials (0.5 mL size, low protein binding) and treated with cold acetone (0.1 mL; LCMS grade) which was pre-cooled at −20° C. Each vial was vortexed for 30 s and incubated at −20° C. for 10 min prior to centrifuge at 4° C. for 10 min at 16,000×g. Its supernatant was carefully discarded, and its pellet which was likely collected at the bottom and side of the tube was treated with 0.1 mL of cold acetone (−20° C.). Its tube was flicked a few times and centrifuged again at 4° C. for 5 min at 16,000×g. Its supernatant was discarded, and the tube was left open for air drying in a fume hood for 30 min. The pellet left in each tube was redissolved in 5% SDS in PBS pH 7.4 (20 mL) and used for its characterization by reaction with TCO-Cy5 dye and SDS-PAGE gel analysis.

SDS-PAGE gel analysis was performed as set forth in Example XII. FIG. 22 shows photographs of an SDS-PAGE gel silver stained (left side) and fluorescently detected (right side). An aliquot of lysate proteins which were photoreacted with DBCO-PEG₄-mTz at 365 nm was treated with DL650 NHS ester (20 eq) or TCO-Cy5 (30 eq) at 25° C. overnight prior to SDS-PAGE. Lane a-c (proteins+DL650 NHS): loaded amounts: 2× (a), 3× (b), 5× (c). Lane d-f (proteins+TCO-Cy5): loaded amounts: 2× (d), 3× (e), 5× (f). Legends for lane g-l (controls): An aliquot of the lysate proteins which were exposed to 365 nm light otherwise without DBCO-PEG₄-mTz was treated with DL650 NHS ester (20 eq) or TCO-Cy5 (30 eq) at 25° C. overnight prior to SDS-PAGE analysis. Lane g-i (proteins+DL650 NHS): loaded amounts: 2× (g), 3× (h), 5× (i). Lane j-l (proteins+TCO-Cy5): loaded amounts: 2× (j), 3× (k), 5× (l). The gel image acquired at Cy5 channel (FIG. 22, right side) shows a set of numerous fluorescent bands which are attributable to either DL650-labeled proteins (lane a-c; g-i) or Cy5-clicked proteins (lane d-f). Those latter bands (lane d-f), which are pointed by arrows, are attributable to mTz-modified proteins given their mTz-specific reactivity for clicking with TCO-Cy5 through IEDDA reaction. Their fluorescent bands appear less intense those labeled with DL650 NHS ester exhaustively at lysine because they are labeled only at cysteine which is less frequent in the proteome.

### Example XV

### Photoreaction of Protein Tau with DBCO-PEG₄-mTz

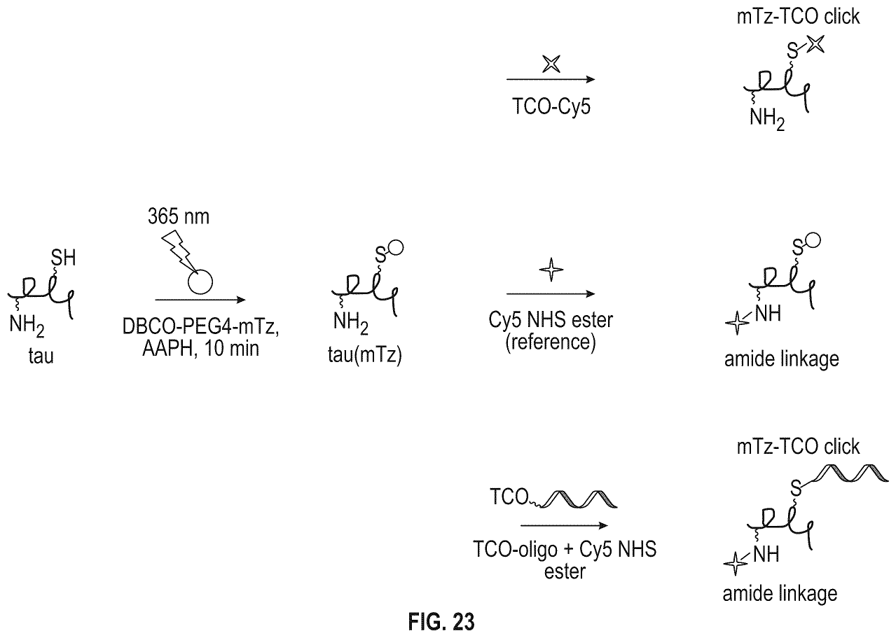

This example describes a representative method of photo thiol-yne chemistry applied in modifying a single target protein. Here, an isoform of protein tau is selected and modified by photoreaction with methyl tetrazine (mTz) at its two cysteine residues. This mTz-modified protein is then characterized by conjugation with TCO-Cy5 or a TCO-terminated oligonucleotide (TCO-oligo) through Inverse Electron Demand Diels-Alder (IEDDA) reaction. A diagram of the reaction scheme is shown in FIG. 23.

First, tau 0N4R (5 μL, 0.25 nmol; Boston Biochemical) in PBS pH 7.4 was treated with a TCEP solution (5 μL, 2.5 mM; 50 eq) and incubated at 5° C. for 2 h. This TCEP-reduced protein was mixed with DBCO-PEG₄-mTz (10 μL, 5 nmol; 20 eq) and AAPH (6.8 μL, 18.5 mM; 500 eq), and its mixture was left in an ice bath for 10 min prior to irradiation at 365 nm using Asahi Spectra Max-350. After 8 min irradiation, its solution was treated with freshly prepared H₂O₂ (2.5 μL, 98 mM) at 5° C. for 1.5 h for inactivating excess TCEP.

Protein purification by acetone precipitation. After photoreaction, unreacted mTz-PEG₄-DBCO was removed from the protein solution in two alternative ways. In one method, an aliquot (13 μL) of the irradiated solution was treated with cold acetone (78 μL; LCMS grade) which was pre-chilled at −20° C., and its precipitated protein was obtained by centrifugation at 16,000×g (5 min, 4° C.) as described in Example XIV. The pellet collected at the bottom of the vial was re-dissolved in 5% SDS in PBS pH 7.4 for characterization by SDS-PAGE analysis.

Protein purification by liquid-liquid extraction. This extraction method was performed by mixing the protein solution (39 μL) with ethyl acetate (350 μL), vortexing for 1 min, leaving in an ice bath for 5 min and spinning it down for a clear separation of two layers. Its organic solvent layer (upper) was carefully discarded while leaving its aqueous layer (lower) in the vial which appeared slightly opaque. This extraction process was repeated two more times with ethyl acetate (350 μL each), affording a protein solution free from unreacted mTz-PEG₄-DBCO. This protein solution was used for characterization by SDS-PAGE analysis.

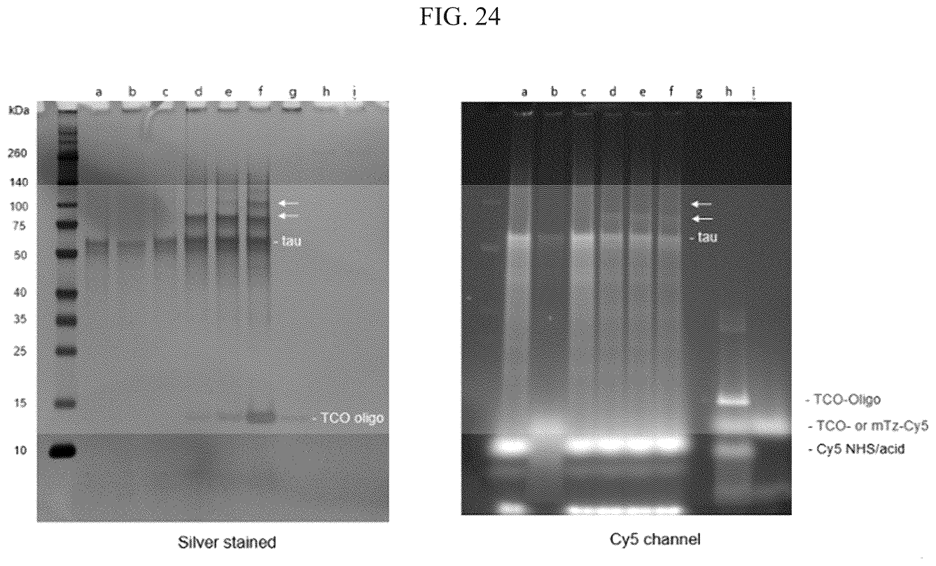

SDS-PAGE analysis was performed as described in Example XII. FIG. 24 shows photographs of an SDS-PAGE gel silver stained (left side) and fluorescently detected (right side). Lane a: tau(mTz)+Cy5 NHS (5 eq); lane b: tau(mTz)+TCO-Cy5 (10 eq); lane c: tau(mTz)+Cy5 NHS (5 eq); lane d-f: tau(mTz)+Cy5 NHS (5 eq)+TCO-oligo(1 eq, d; 2 eq, e; 10 eq, f); lane g: TCO-oligo (1 eq); lane h: TCO-oligo (1 eq)+mTz-Cy5 (5 eq); lane i: mTz-Cy5 alone. Each reaction mixture was shaken at 35° C. for 12 h and characterized by SDS-PAGE analysis. The gel images suggest successful conjugation with TCO-dye (lane b) or TCO-oligonucleotide (lane d-f). The latter case is suggested by two new bands generated, each migrating at ˜75 and 100 kDa, respectively, and thus larger than unmodified tau or TCO-oligonucleotide alone. Of these, the larger band at 100 kDa is possibly indicative of protein conjugation with two oligonucleotides given its higher MW and its band intensity which shows TCO-oligo amount-dependent growth. Overall, this image set is evidently supportive of mTz-modification of tau protein.

### Example XVI

### Presentation of mTz-Modified Tau Protein in Structured Nucleic Acid Particle (SNAP)

Like successful TCO-oligo conjugation, access to mTz-modified protein opens a route for covalent protein attachment to a single TCO-oligo handle presented on the surface of structured nucleic acid particle (SNAP). This example describes a representative method of presenting a single tau protein on the SNAP surface through Inverse Electron Demand Diels-Alder (IEDDA) conjugation.

In a vial was loaded three solutions, each for mTz-modified tau protein (52 pmol, 14 μL), MgCl₂ (4 μL, 200 mM) and 1TCO-presenting SNAP (8.7 pmol, 42 μL). This mixture was shaken at 25° C. while its reaction progress was assessed by monitoring depletion of 1TCO-SNAP which is added at a lower equivalent to mTz-modified tau protein. This was performed by removing a small fraction (4 μL), reacting with excess mTz-Cy5 at 25° C. and performing SDS-PAGE analysis. Full depletion of 1TCO-SNAP was indicated by the absence of fluorescent bands that are attributed to Cy5-clicked TCO-oligonucleotide. The crude SNAP-tau mixture was purified by spin filtration through a membrane filter tube (Amicon™ MWCO 100,000, 0.5 mL capacity). This was performed by diluting its mixture with 1×origami buffer (0.4 mL), loading it into the membrane tube and centrifuging at 8000 rpm for 5 min (Eppendorf 5430/5430R). The filtrate in the lower tube was discarded. The concentrate in the upper tube contained the conjugation product, SNAP-tau protein, and it was collected by spinning at 8000 rpm for 5 min. This process afforded 70 μL of SNAP-tau conjugate at a concentration of 434 ng/μL (99 nM; 75.6% recovery).

### Example XVII

### Photomodification of Cell Lysate Proteins with propargyl-PEG₁₂-azide

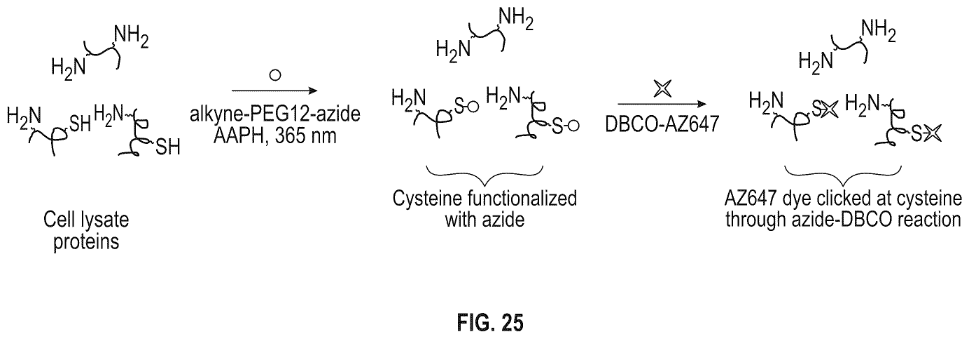

This example describes a representative method for performing azide modification of proteins through photo thiol-yne chemistry using proteins present in cell lysates. In this example, proteins are modified by photoreaction with propargyl-PEG₁₂-azide at cysteine thiol. Production of such azide-modified proteins is verified by azide-specific conjugation with a DBCO-functionalized dye molecule through strain-promoted azide-alkyne click (SPAAC) conjugation. FIG. 25 shows a diagram of the reaction scheme.

As described in Example XIII, lysate proteins were treated with TCEP for reducing disulfide bonds to free cysteine thiols. After this treatment, a protein solution (25 μL, 0.26 nmol of proteins) was mixed with AAPH (2.8 μL, 5 mg/mL PBS; 200 eq) and propargyl-PEG₁₂-azide (12.3 μL, 4.2 mM; 200 eq) and its mixture was irradiated at 365 nm for 10 min in Asahi Spectra Max-350. After irradiation, its solution was treated with iodoacetamide (IAA; 12.8 μL, 10 mM; 500 eq) and shaken (650 rpm) at 25° C. for 30 min. After its IAA treatment, excess reagents in the protein solution were removed through acetone precipitation as described in Example XIV. The pellet left in the tube was redissolved in 5% SDS in PBS pH 7.4 (25 μL) and used for its characterization by SDS-PAGE gel analysis as set forth in Example XIII.

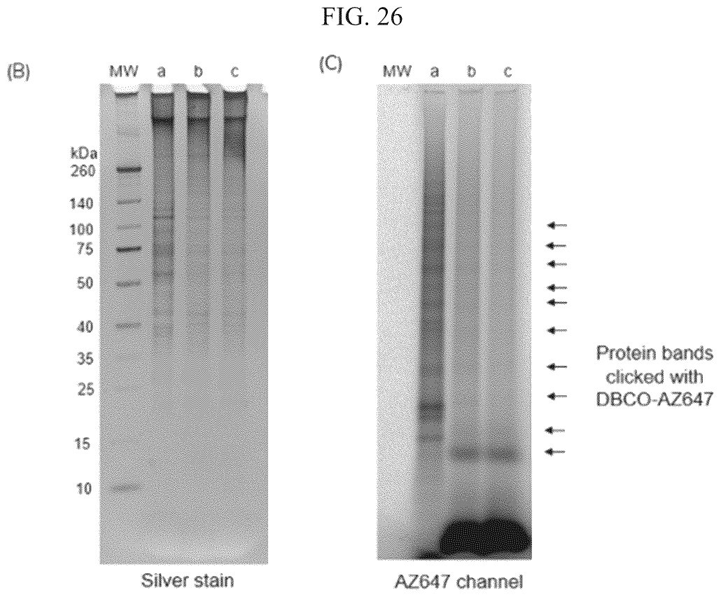

FIG. 26 shows photographs of an SDS-PAGE gel silver stained (left side) and fluorescently detected (right side). Legends for lane a: lysate proteins (unmodified)+DL650 NHS ester (20 eq), 25° C., 12 h; lane b-c: lysate proteins (photoreacted with alkyne-PEG12-azide)+DBCO-AZ647 (10 eq for lane b or 20 eq for lane c), 25° C., 12 h. Production of azide-modified proteins in cell lysate is supported by fluorescent bands obtained after post conjugation with AZ647 (black arrows; lane b, c). Though not identical, most of those fluorescent bands (lane b, c) are comparable to those observed from lysine-labeled fluorescent bands (lane a) in protein size (MW) and diversity. However, some of these bands appear stronger in intensity as shown in the band at ˜10-15 kDa.

### Example XVII

### Photo Crosslinking of Cell Lysate Proteins with DBCO-PEG-₈-biotin

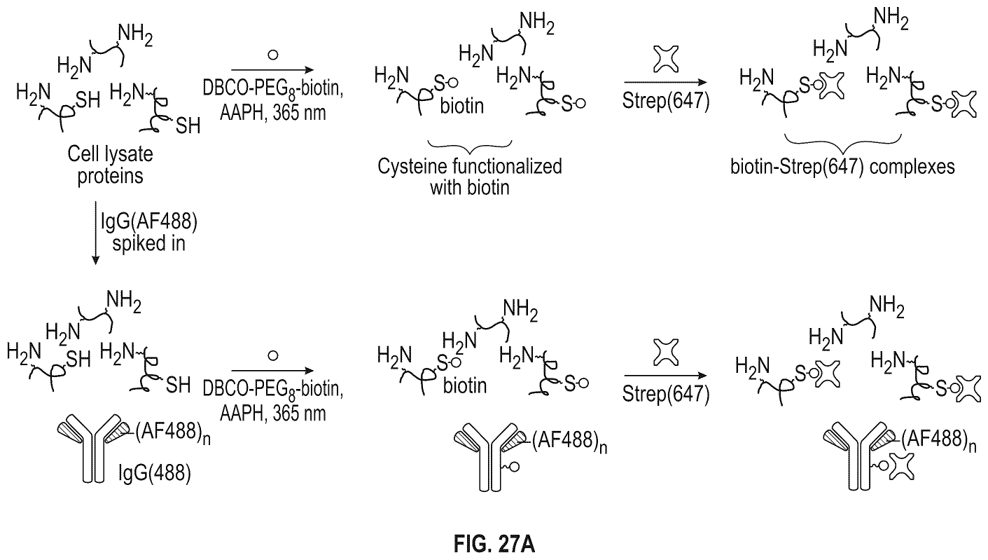

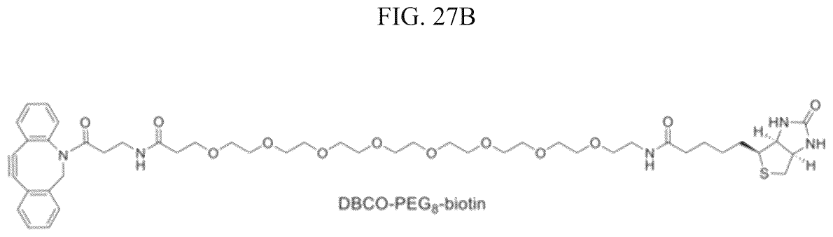

This example describes a representative method for biotin modification of proteins present in cell lysates through photo thiol-yne chemistry. In this example, proteins are modified by photoreaction with DBCO-PEG₈-biotin at cysteine thiol. Production of such biotin-modified proteins is verified by their complexation with AZ647-labeled streptavidin. A diagram of the reaction scheme is shown in FIG. 27A.

A solution of lysate proteins pre-treated with TCEP (25 mL, 0.26 nmol of proteins) was mixed with AAPH (4.2 μL, 5 mg/mL PBS; 300 eq) and DBCO-PEG₈-biotin (23.8 μL, 2.2 mM; 200 eq; FIG. 27B). This mixture was irradiated at 365 nm for 10 min in Asahi Spectra Max-350 and treated with iodoacetamide (IAA; 12.8 μL, 10 mM; 500 eq) at 25° C. for 30 min. After IAA treatment, excess reagents in the protein solution were removed through acetone precipitation as described in Example XIV. The pellet left in the tube was redissolved in 5% SDS in PBS pH 7.4 (25 μL) and used for its characterization by SDS-PAGE gel analysis.

In addition, this biotin modification is applicable to cell lysate proteins spiked with an external protein which is added to serve as a reference. This is illustrated by cell lysate proteins spiked with IgG(labeled with AF488 fluorophore) (10% molar equiv). First, the IgG(AF488)-spiked cell lysate proteins were pre-treated with TCEP (25 μL, 0.26 nmol of proteins) for 2 h at room temperature prior to mixing with AAPH (4.2 μL, 5 mg/mL PBS; 300 eq) and DBCO-PEG₈-biotin (23.8 μL, 2.2 mM; 200 eq). This mixture was irradiated at 365 nm for 10 min and treated with iodoacetamide (IAA; 12.8 μL, 10 mM; 500 eq) at 25° C. for 30 min. After its IAA treatment, excess reagents in the protein solution were removed through acetone precipitation. The pellet left in the tube was redissolved in 5% SDS in PBS pH 7.4 (25 μL) and characterized by SDS-PAGE gel analysis.

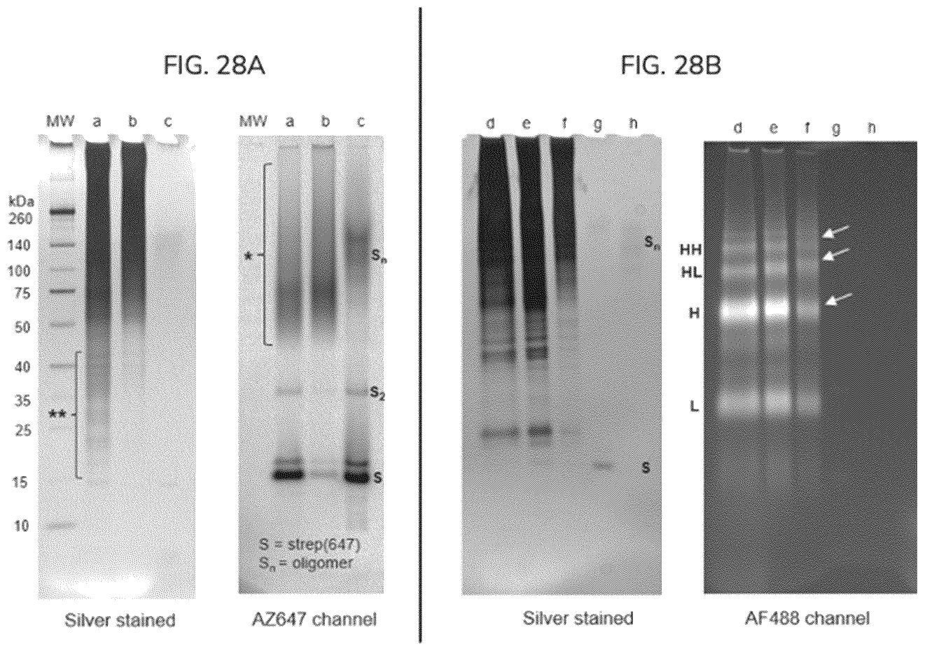

SDS-PAGE gel analysis was performed as set forth in Example XIII. FIG. 28A shows photographs of an SDS-PAGE gel silver stained (left side) and fluorescently detected (right side), characterizing biotin-modified proteins of cell lysate proteins by complexation with streptavidin(AZ647). Legends for lane a: lysate proteins (biotin photoreacted)+streptavidin(647) (1 eq), heated; lane b: lysate proteins (biotin photoreacted)+streptavidin(647) (1 eq), not heated; lane c: streptavidin(647) alone (1 eq), heated. Production of biotin-phtoreacted proteins in cell lysate is supported by fluorescent protein bands (* marked) that result from their complexation with streptavidin(647) (lane a, b; FIG. 28A). Such complexation is verifiable through protein denaturation, which is achieved by heating at 90° C. for 10 min (lane a) in which some of these complexes are dissociated to non-fluorescent bands (** marked). Other complexes which are located at higher MW bands are otherwise able to survive under such a denaturing condition, suggesting higher stability.

FIG. 28B shows photographs of an SDS-PAGE gel silver stained (left side) and fluorescently detected (right side), characterizing biotin-modified proteins of IgG(488)-spiked cell lysate proteins by complexation with streptavidin(647). lane d: lysate proteins/IgG(488) (biotin photoreacted), heated; lane e: lysate proteins/IgG(AF488) (biotin reacted)+streptavidin(647) (1 eq), heated; lane f: lysate proteins/IgG(AF488) (biotin photoreacted)+streptavidin(647) (1 eq), not heated; lane g: streptavidin(647) alone (1 eq), heated; lane h: streptavidin(647) alone (1 eq), not heated. Abbreviations: H=heavy chain of IgG, L=light chain of IgG. Evidence for biotin photoreaction is also observed in the IgG(AF488)-spiked cell lysate sample (FIG. 28B). Lane f shows that IgG bands (detected at 488 channel) are shifted to higher MW species (pointed by white arrows) upon streptavidin(647) treatment compared to those in lane d where no streptavidin(647) is added or those in lane e where proteins are denatured in part due to heating.

### Example XIX

### Modification of Anti-DTR IgG₁ with Dibenzocyclooctyne (DBCO)-PEG₈-methyltetrazine (mTz)

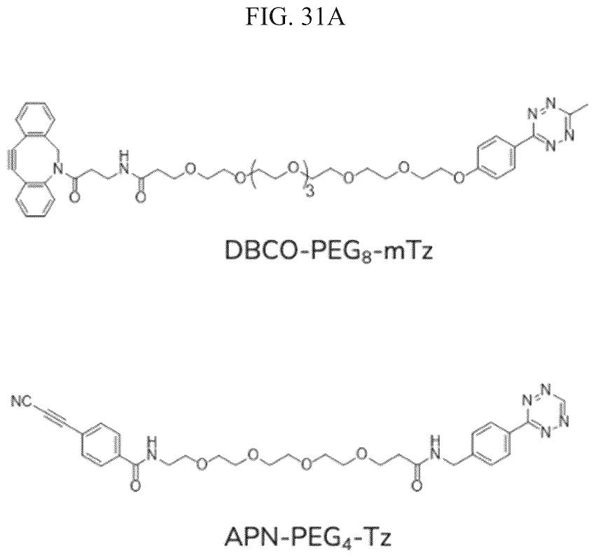

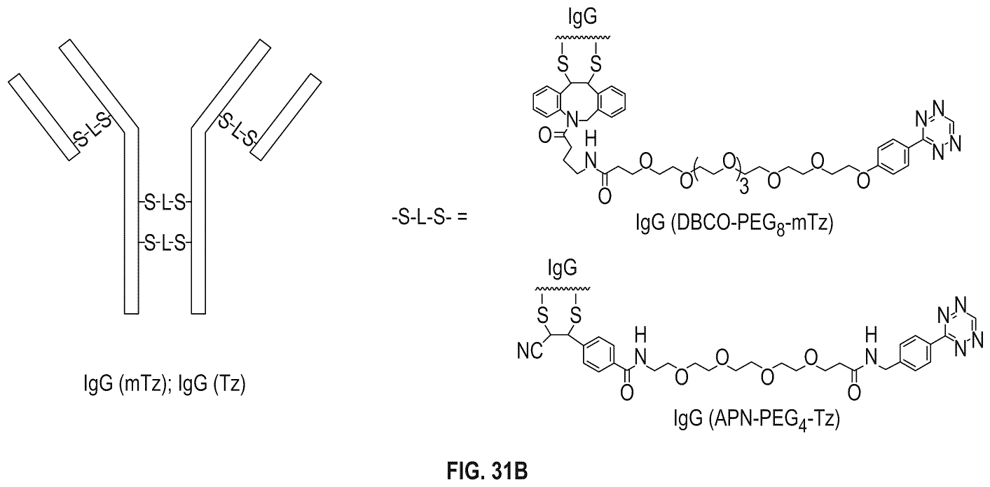

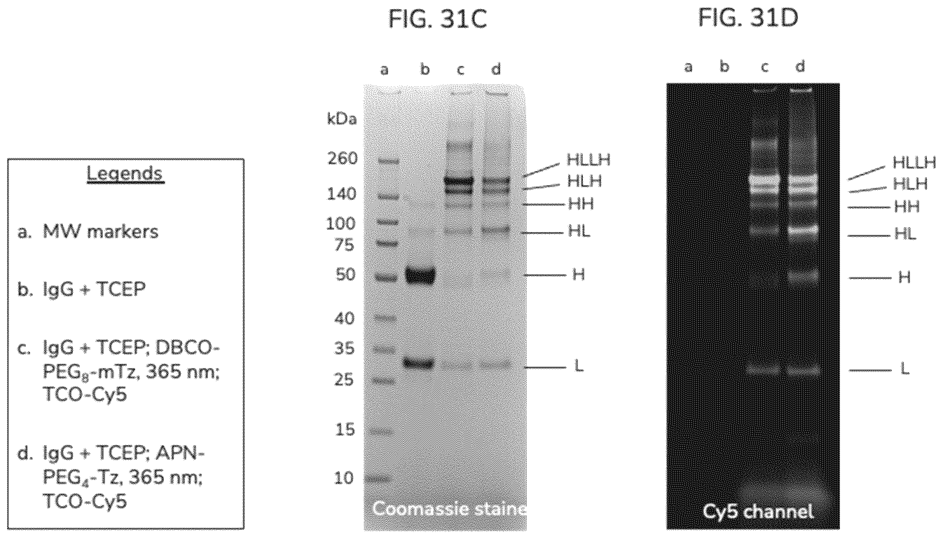

Anti-DTR IgG1 antibody (Nautilus Biotechnology, San Carlos, CA) dissolved in phosphate buffered saline (PBS) pH 7.2 (1 mg/mL) was reacted with tenfold molar excess of TCEP at 5° C. for overnight or 12 hours to produce reduced IgG₁ antibody. The reduced IgG₁ antibody was then reacted with DBCO-PEG₈-mTz (BroadPharm, Cat# BP-25742; see FIG. 31A) in the presence of 15-fold molar equivalent of lithium phenyl-2,4,6-trimethylbenzoylphosphinate (LAP) (Cat# 900889; Sigma-Aldrich) under irradiation with light at 365 nm for 7 minutes to produce an antibody conjugate modified with mTz residues, IgG(DBCO-PEG₈-mTz), (see FIG. 31B, upper right). Following the photoreaction, the antibody conjugate was optionally treated with 4.2 mM H₂O₂ for 2 hours at room temperature to reoxidize any cysteine thiols that were not modified by attachment to the DBCO moiety. The IgG(mTz) conjugate was then treated with TCO-Cy5 (10 molar equivalent, 25° C., 15 hours) for dye conjugation at mTz residues and its dye conjugation products were evaluated using SDS-PAGE under non-reducing conditions.

Results are shown in FIG. 31C (Coomassie stain) and in FIG. 31D (fluorescence in Cy5 channel) for the SDS-PAGE loaded with molecular weight standards (lane a), anti-DTR IgG1 (TCEP reduced; lane b), anti-DTR IgG1+TCEP; DBCO-PEG₈-mTz, 7 minutes, 365 nm; H₂O₂ (lane c). Locations are indicated for tetrameric antibody (LHHL), dimeric antibody (HL), heavy chain (H), light chain (L) and modified antibody species associated with them. Anti-DTR IgG1 conjugates having the mTz moiety attached via a synthetic bridge or a crosslink are indicated by fluorescence attributed to TCO-Cy5 conjugated at mTz (lane c, FIG. 31D).

These results demonstrated that anti-DTR IgG1 antibody reacted with DBCO-PEG₈-mTz and LAP (photo catalyst) under irradiation with UVA light and yielded its conjugates having an mTz moiety and synthetic bridges between antibody subunits that maintained association of tetramers (LHHL) and dimers (HL).

### Example XX

### Modification of Anti-DTR IgG₁ with 3-(4-Acylphenyl)propiolonitrile (APN)-PEG₄-tetrazine (Tz)

Anti-DTR IgG1 antibody (Nautilus Biotechnology, San Carlos, CA) dissolved in borate buffered saline (BBS) pH 8.2 (1 mg/mL) was reacted with tenfold molar excess of TCEP at 5° C. for overnight or 12 hours to produce reduced IgG₁ antibody. The reduced IgG₁ antibody was then reacted with APN-PEG₄-Tz (Conju-Probe, Cat# CP-8010-25mg; see FIG. 31A) in the presence of 15-fold molar equivalent of lithium phenyl-2,4,6-trimethylbenzoylphosphinate (LAP) (Cat# 900889; Sigma-Aldrich) under irradiation with light at 365 nm for 7 minutes to produce an antibody conjugate modified with Tz residues, IgG(APN-PEG₄-Tz), (see FIG. 31B, lower right). Following the photoreaction, the antibody conjugate was optionally treated with 4.2 mM H₂O₂ for 2 hours at room temperature to reoxidize any cysteine thiols that were not modified by attachment to the APN moiety. The IgG(Tz) conjugate was then treated with TCO-Cy5 (10 molar equivalent, 25° C., 15 hours) for dye conjugation at Tz residues and its dye conjugation products were evaluated using SDS-PAGE under non-reducing conditions.

Results are shown in FIG. 31C (Coomassie stain) and in FIG. 31D (fluorescence in Cy5 channel) for the SDS-PAGE loaded with molecular weight standards (lane a), anti-DTR IgG1 (TCEP reduced; lane b), anti-DTR IgG1+TCEP; APN-PEG4-Tz, 7 minutes, 365 nm; H₂O₂ (lane d). Locations are indicated for tetrameric antibody (LHHL), dimeric antibody (HL), heavy chain (H), light chain (L) and modified antibody species associated with them. Anti-DTR IgG1 conjugates having the Tz moiety attached via a synthetic bridge or a crosslink are indicated by fluorescence attributed to TCO-Cy5 conjugated at Tz (lane d, FIG. 31D).

These results demonstrated that anti-DTR IgG1 antibody reacted with APN-PEG₄-Tz and LAP (photo catalyst) under irradiation with UVA light and yielded its conjugates having a Tz moiety and synthetic bridges between antibody subunits that maintained association of tetramers (LHHL) and dimers (HL).

### Example XXI

### Click Conjugation of (Methyl)tetrazine-modified IgG₁ with Trans-cyclooctyne (TCO)-Presenting Single Nucleic Acid Particle (SNAP)

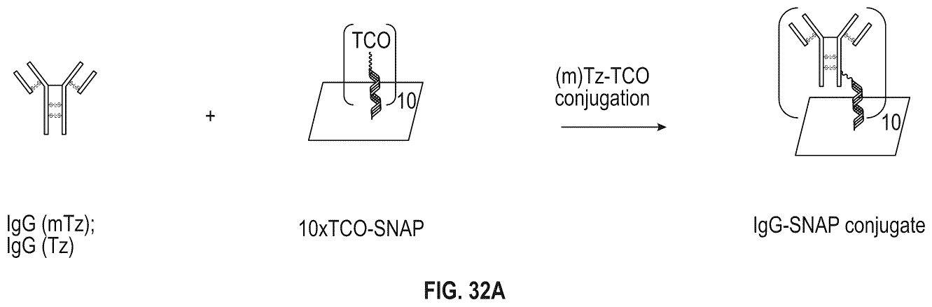

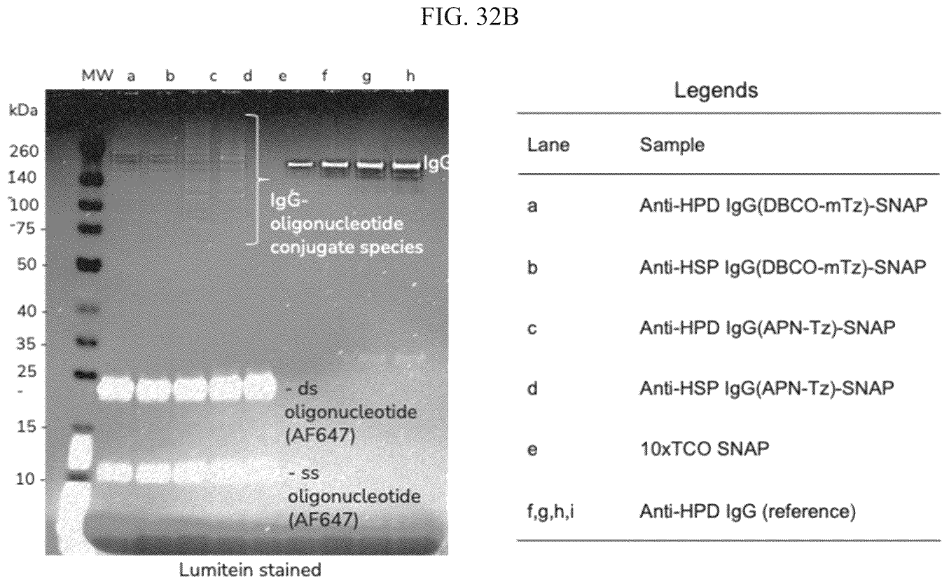

Two IgG₁ antibodies, anti-HPD IgG and anti-HSP IgG, were each photo modified with either DBCO-PEG₈-mTz or APN-PEG₄-Tz as depicted in FIG. 31B to produce four types of modified IgG antibodies that include anti-HPD IgG (mTz), anti-HSP IgG (mTz), anti-HPD IgG (Tz) and anti-HSP IgG (Tz). Each antibody was prepared at 1.0-1.4 mg/mL in phosphate buffered saline (PBS) pH 7.4 supplemented with 12 mM MgCl2 and added at a 40-fold molar excess to 10×TCO-SNAP (ten TCO residues per particle) prepared in origami buffer (759 ng/μL). Each of their mixtures was shaken (650 rpm) at 25° C. for 15 hours to produce IgG-SNAP conjugate through mTz-TCO reaction (FIG. 32A). Following their reaction, the product of IgG-SNAP conjugate contained in each mixture was obtained by fractionation in size exclusion chromatography (SEC)-HPLC (column: Shodex OHpak; eluent=origami buffer; flow rate=0.3 mL/min). Their products were evaluated using SDS-PAGE under non-reducing conditions.

Results are shown in FIG. 32B for an SDS-PAGE gel (Lumitein™ stained and imaged in SYPRO Ruby channel) loaded with anti-HPD IgG(DBCO-mTz)-SNAP (lane a), anti-HSP IgG(DBCO-mTz)-SNAP (lane b), anti-HPD IgG(APN-Tz)-SNAP (lane c), anti-HSP IgG(APN-Tz)-SNAP (lane d) and 10×TCO-SNAP (lane e). IgG and its associated species which are conjugated with TCO-oligonucleotide are indicated by protein-specific bands (stained by Lumitein™) and attributed to IgG conjugation at SNAP.

These results demonstrated that each of two IgG1 antibodies reacted with DBCO-PEG₈-mTz or APN-PEG₄-Tz under a photo catalytic condition (LAP, 365 nm, 7 min) and yielded its conjugates having mTz or Tz moieties through synthetic bridges (FIG. 31B). Furthermore, those mTz or Tz moieties attached in each IgG antibody displayed TCO-specific reactivity and conjugation to a TCO-presenting SNAP.

What is claimed is:

1. A method of modifying an antibody comprising
reacting cysteines of an antibody with an alkyne moiety of a modifying reagent, wherein the alkyne moiety reacts with the cysteines to form a moiety of Formula I: 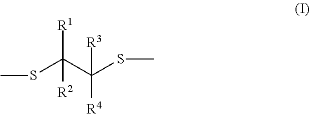
wherein S comprises the sulfur moieties of the cysteines, and
wherein R¹, R², R³, and R⁴ are each independently selected from the group consisting of hydrogen, halo, alkyl, alkenyl, alkynyl, aryl, heteroalkyl, heteroalkenyl, heteroalkynyl, heteroaryl, and optionally substituted variants thereof.

2. The method of claim 1, wherein the reacting comprises irradiating the alkyne moiety of the modifying reagent with light.

3. The method of claim 2, wherein the light comprises UV energy.

4. The method of claim 1, wherein the alkyne moiety is present in a strained ring moiety of the modifying reagent.

5. The method of claim 1, wherein the alkyne moiety is present in a cyclooctyne, cycloheptyne, cyclohexyne or cyclopentyne moiety of the modifying reagent.

6. The method of claim 5, wherein the alkyne moiety is present in a dibenzocyclooctyne moiety of the modifying reagent.

7. The method of claim 1, wherein the alkyne moiety is present in a linear hydrocarbon moiety of the modifying reagent.

8. The method of claim 1, further comprising contacting the antibody with a reducing agent prior to or during the reacting, whereby the sulfur moieties are reduced.

9. The method of claim 1, wherein the alkyne moiety reacts with a sulfur moiety on a heavy chain of the antibody and a sulfur moiety on a light chain of the antibody, thereby synthetically crosslinking the heavy chain to the light chain.

10. The method of claim 9, wherein an alkyne moiety reacts with a sulfur moiety on a second heavy chain of the antibody and a sulfur moiety on a second light chain of the antibody, thereby synthetically crosslinking the second heavy chain to the second light chain.

11. The method of claim 1, wherein the alkyne moiety reacts with a sulfur moiety on a first heavy chain of the antibody and a sulfur moiety on a second heavy chain of the antibody, thereby synthetically crosslinking the first heavy chain to the second heavy chain.

12. The method of claim 11, wherein an alkyne moiety reacts with a second sulfur moiety on the first heavy chain of the antibody and a second sulfur moiety on the second heavy chain of the antibody, thereby forming a second synthetic bridge between the first heavy chain and the second heavy chain.

13. The method of claim 1, wherein the alkyne moiety reacts with a first sulfur moiety on a heavy chain of the antibody and a second sulfur moiety on the heavy chain of the antibody, thereby forming an intrachain synthetic crosslink in the heavy chain.

14. The method of claim 1, wherein the antibody comprises a Fab, Fab′, F(ab′)2, single-chain variable (scFv), di-scFv, tri-scFv, or microantibody.

15. The method of claim 1, wherein the modifying reagent comprises an attachment moiety.

16. The method of claim 15, further comprising attaching the antibody to a solid support via the attachment moiety.

17. The method of claim 15, further comprising attaching the antibody to a label via the attachment moiety.

18. The method of claim 15, further comprising attaching the antibody to a structured nucleic acid particle via the attachment moiety.

19. The method of claim 1, wherein the modifying reagent comprises a label moiety.

20. An antibody, comprising a pair of cysteines crosslinked via a moiety of Formula I: 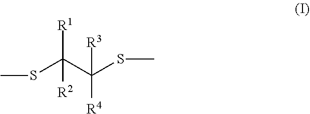
wherein S comprises the sulfur moieties of the cysteines, and
wherein R¹, R², R³, and R⁴ are each independently selected from the group consisting of hydrogen, halo, alkyl, alkenyl, alkynyl, aryl, heteroalkyl, heteroalkenyl, heteroalkynyl, heteroaryl, and optionally substituted variants thereof.


SEQUENCE LISTING (1 sequence)

SEQ ID NO: 1 - DNA, 41 bp, synthetic construct
  source 1..41: mol_type = other DNA
tgtgggagttggatgggtaggagtggagatggaggtgagtg